(19) United States
(12) Patent Application Publication
(10) Pub. No.: US 2023/0321217 A1
(43) Pub. Date: Oct. 12, 2023

(54) COMPOSITIONS COMPRISING COMPLEXES DISPLAYING ANTIGENS AND METHODS OF USING THE COMPOSITIONS

(71) Applicants: Yoshihiro Kawaoka, Middleton, WI (US); Peter J. Halfmann, Madison, WI (US); Ravi Kane, Roswell, GA (US); Steven J. Frey, Conshohocken, PA (US); Gabriele Neumann, Madison, WI (US); Shiho Chiba, Madison, WI (US); Ana Luisa Stringari de Castro, Arlington, MA (US); Kathryn E. Loeffler, Atlanta, GA (US)

(72) Inventors: Yoshihiro Kawaoka, Middleton, WI (US); Peter J. Halfmann, Madison, WI (US); Ravi Kane, Roswell, GA (US); Steven J. Frey, Conshohocken, PA (US); Gabriele Neumann, Madison, WI (US); Shiho Chiba, Madison, WI (US); Ana Luisa Stringari de Castro, Arlington, MA (US); Kathryn E. Loeffler, Atlanta, GA (US)

(21) Appl. No.: 17/936,194

(22) Filed: Sep. 28, 2022

Related U.S. Application Data
(60) Provisional application No. 63/299,787, filed on Jan. 14, 2022.
(60) Provisional application No. 63/249,409, filed on Sep. 28, 2021.

(51) Int. Cl. A61K 39/215; A61K 9/51; A61P 31/14

(52) U.S. Cl. CPC A61K 39/215; A61K 9/5169; A61P 31/14; A61K 2039/6075

(57) ABSTRACT

A nanoparticle that displays a coronavirus spike protein or a portion thereof on its surface, and methods of making and using the nanoparticle, are provided.

### CROSS-REFERENCE TO RELATED APPLICATIONS

This application claims the benefit of the filing date of U.S. Application No. 63/249,409, filed on Sep. 28, 2021, and U.S. Application No. 63/299,787, filed on Jan. 14, 2022, the disclosures of which are incorporated by reference herein.

### BACKGROUND

The COVID-19 pandemic continues to rage worldwide with more than a million estimated fatalities already and global economic; costs in the hundreds of billions of dollars. Without an effective vaccine, SARS-CoV-2 will continue to strain the world’s economies and devastate many facets of society. Various vaccines such as nucleic acid-based vaccines, viral vector-based vaccines, subunit vaccines, and inactivated vaccines are in different stages of clinical trials (Krammer et al., 2020). The contemporary vaccine candidates focus on stimulating protective immune responses to the spike (S) protein of SARS-CoV-2, the protein that facilitates viral entry by binding to the angiotensin-converting enzyme 2 (ACE2) receptor on the surface of host cells (Yang et al., 2020). Neutralizing antibodies that target the spike protein could, therefore, play a role in protecting the host from this viral infection (Addetia et al., 2020; Walls et al., 2020).

Numerous vaccine candidates are therefore being developed to provide protection against SARS-CoV-2, including nucleic acid-based vaccines, viral vector-based vaccines, subunit vaccines, and inactivated vaccines (Krammer, 2020). Most of the vaccines in development aim to elicit a protective immune response that targets the spike (S) protein of SARS-CoV-2. The receptor binding domain (RBD) of the trimeric S protein, which initiates infection by binding to the host cell receptor angiotensin-converting enzyme 2 (ACE2) (Yang et al., 2020), is the primary target of neutralizing antibodies elicited by vaccination or infection. These antibodies are able to neutralize the virus by either binding to the receptor binding motif to directly inhibit binding to ACE2 or by binding to the RBD in a manner that locks it in an unstable state, leading to dissociation of the trimer.

Although several vaccines have been approved for clinical use and have demonstrated high effectiveness against the original strain of the virus, the recently emerged “variants of concern” are better able to escape neutralization by vaccine-induced humoral immunity, leading to a decrease in vaccine potency. The emergence of variants has motivated the design and testing of booster shots that can provide protection against these new circulating strains. While this is a reasonable near-term approach, it would be desirable to develop a vaccine that would provide broad protection against emerging SARS-CoV-2 variants.

Standard vaccine platforms may provide the first generation of vaccines against SARS-CoV-2, however, nanotechnology (Shin et al., 2020) has the potential to offer new and improved vaccine platforms against diseases caused by emerging viruses including SARS-CoV-2. Nanoparticles such as virus-like particles (VLPs) are ideal scaffolds for antigen display, because they emulate many of the properties of natural viruses including their size and geometry (Shin et al., 2020; Bachmann et al., 2010; Frietze et al., 2016; Plummer et al., 2011). Moreover, the multivalent display of antigens from nanoscale scaffolds can result in the effective clustering of B cell receptors and greatly enhance their immunogenicity Bachmann et al., 1997) A recent report confirmed that the S protein displayed on a nanoscale scaffold was more immunogenic in mice than the S protein administered alone, but the study used two sequential immunizations (prime + boost) and did not test protective efficacy in mice challenged with SARS-CoV-2 (Zhang et al., 2020).

While S-targeting vaccines in current use are based on the full-length S protein, vaccines based on the RBD4 (Lederer et al., 2020; Tan et al., 2021; Kang et al., 2021; Cohen et al., 2021; Saunders et al., 2021), the primary target of neutralizing antibodies, are worth exploring. Moreover, parts of the RBD are conserved, not just between the SARS-CoV-2 variants, but also between SARS-CoV-2 and SARS-CoV-1. Antibodies binding to these conserved regions have already been shown to neutralize SARS-CoV-2 as well as SARS-CoV-1 pseudoviruses (Lv, et al., 2020; Pinto et al., 2020). Lederer et al. (2020) recently compared two RBD vaccine platforms – an mRNA vaccine and recombinant RBD formulated with Addavax, an MF59-like adjuvant - and reported that the mRNA vaccines were superior at eliciting SARS-CoV-2 specific germinal center B cell responses. This work, however, used monomeric recombinant RBD; in contrast, several groups have reported robust protective immune responses upon vaccination with RBDs presented multivalently from nanoparticle scaffolds (Tan et al., 2021; Cohen et al., 2021; Saunders et al., 2021) and at least one such candidate is in clinical trials (Sheridan, 2021). Tan et al. (2021) used SpyCatcher/SpyTag chemistry for the assembly of the SARS-CoV-2 RBD on SpyCatcher003-mi3 nanoparticles and showed that a prime-boost regimen elicited strong neutralizing antibody responses in mice and pigs that were superior to those in convalescent human sera. Cohen et al. (2021) designed mosaic nanoparticles co-displaying SARS-CoV-2 RBD along with RBDs from other animal betacoronaviruses that elicited antibodies with cross-reactive recognition of heterologous RBDs. Kang et al. (2021) designed three different RBD-conjugated nanoparticles and reported higher neutralizing antibody titers against authentic SARS-CoV-2 virus for the resulting antisera relative to those for mice immunized with monomeric RBD (Kang et al., 2021). Recently, Sanders et al.8 showed that macaque immunization with a multimeric SARS-CoV-2 RBD nanoparticle elicited cross-neutralizing antibody responses against SARS-CoV-2, the variants of concern (B.1.1.7, P.1, and B.1.351), SARS-CoV-1, and bat coronaviruses.

### SUMMARY

The COVID-19 pandemic continues to wreak havoc as worldwide SARS-CoV-2 infection, hospitalization, and death rates climb unabated. Effective vaccines remain the most promising approach to counter SARS-CoV-2. Yet, while promising results are emerging from COVID-19 vaccine trials, the need for multiple doses and the challenges associated with the widespread distribution and administration of vaccines remain concerns. As described herein, in one embodiment, the coat protein of the MS2 bacteriophage was employed to generate nanoparticles displaying multiple copies of the SARS-CoV-2 spike (S) protein. The use of these nanoparticles as vaccines generated high neutralizing antibody titers and protected Syrian hamsters from a challenge with SARS-CoV-2 after a single immunization with no infectious virus detected in the lungs. This nanoparticle-based vaccine platform thus provides protection after a single immunization and may be broadly applicable for protecting against SARS-CoV-2 and future pathogens with pandemic potential.

Also described herein is a composition in which the protective immune response is targeted to a more conserved region of the S protein. As disclosed herein, the efficacy against SARS-CoV-2 of an immunogen based on the conserved S2 subunit of the S protein was demonstrated. Hamsters immunized with S2-based constructs conjugated to virus-like particles (VLPs) were protected from a challenge with SARS-CoV-2. Moreover, the immunization elicited broadly cross-reactive antibodies that recognized the spike proteins of the SARS-CoV-2 variant B.1.351, SARS-CoV-1, and the four endemic human coronaviruses. These results provide a framework for designing S2-based vaccines that elicit broad protection against coronaviruses. In particular, virus-like particles (VLPs) that multivalently displayed either the S2 subunit of the SARS-CoV-2 spike protein or an S2 variant designed to prevent potential proteolytic cleavage at the S2′ cut site were prepared. After characterizing the VLP-S2 constructs in vitro, they were used to vaccinate hamsters. The immunized hamsters showed significantly lower viral titers in the lungs and nasal turbinates after challenge with SARS-CoV-2 compared to control hamsters. Sera from the hamsters immunized with VLP-S2 showed substantial cross-reactive IgG antibody recognition of the spike proteins of SARS-CoV-2 variants, SARS-CoV-1, and the four endemic human coronaviruses. Most importantly, immunization also significantly reduced virus titers in the respiratory tissues of hamsters challenged with SARS-CoV-2 variants B.1.351 (beta), B.1.617.2 (delta), and BA.1 (omicron) as well as from a pangolin coronavirus. Immunization inhibited virus replication in the lungs of VLP-S2-vaccinated mice challenged with a mouse-adapted SARS-CoV-2 and elicited a broad neutralizing response. Thus, S2-based immunogens are an attractive approach to design broadly protective coronavirus vaccines.

In one embodiment, vaccine constructs based on 24 subunit, 60 subunit, or 120 subunit containing particles such as SpyCatcher-mi3 nanoscaffolds, or ferritin, e.g., Heliobacter pylori ferritin, or Aquifex aeoli lumozine synthase scaffolds, which have been used to display a variety of different antigens through SpyTag-SpyCatcher conjugation (Bruun et al., 2018), including the SARS-CoV-2 RBD (Tan et al., 2021; Kang et al., 2021). By addition of a SpyTag to the C terminus of RBD, multiple copies of the RBD were irreversibly linked to each SpyCatcher-mi3 particle. The efficacy of the vaccine construct was then tested against a panel of variants. Immunization studies demonstrated the production of a strong and broadly cross-reactive humoral response against SARS-CoV-2 and SARS-CoV-1. Furthermore, the immunization elicited high neutralizing antibody titers, not just against an early isolate of SARS-CoV-2, but also against four important “variants of concern” including the delta variant (B.1.617.2).

Thus, the present disclosure provides for a general platform for nanoparticle-based antigen display that could provide protection against SARS-CoV-2 after a single immunization.

In one embodiment, a nanoparticle is provided that displays a coronavirus spike protein or a portion thereof on its surface, wherein the nanoparticle comprises a fusion polypeptide comprising the coronavirus spike protein or an antigenic portion thereof linked to a first portion of a fibronectin binding protein comprising an aspartic acid residue that is covalently linked to a lysine residue in a second portion of the fibronectin binding protein via an isopeptide bond.

In one embodiment, a virus like particle (VLP) is provided that displays a coronavirus spike protein or a portion thereof, wherein the VLP comprises a coat protein of Fiersviridae comprising a first biotinylated, which first peptide is bound to streptavidin, e.g., divalent, trivalent or tetravalent (or more), that is bound to a second biotin that is bound to a second biotinylated peptide that is linked to the spike protein or a portion thereof that is also linked to trimerization domain, e.g., a T4 fibritin trimerization domain, a collagen trimerization domain, such as a collagen XV or XVIII tnmerization domain, an influenza HA trimerization domain, a GCN4 trimerization domain. RSV F protein trimerization domain. HIV gp120 trimerization domain, or a EML2 trimerization domain..

In one embodiment, a nucleic acid vector is provided encoding a fusion polypeptide comprising a first coat protein of a Fiersviridae and a second coat protein of a Fiersviridae comprising a first peptide that is capable of being biotinylated.

In one embodiment, a nucleic acid vector is provided encoding a fusion polypeptide comprising a coronavirus spike protein or portion thereof, a peptide capable of being biotinylated, and a trimerization domain.

In one embodiment, isolated fusion polypeptide is provided comprising a first coat protein of a Fiersviridae and a second coat protein of a Fiersviridae comprising a first biotinylated peptide.

In one embodiment, isolated fusion polypeptide is provided comprising a coronavirus spike protein or portion thereof, a biotinylated peptide, and a trimerization domain.

Also provided, in one embodiment, the present disclosure provides for a general platform for nanoparticle-based antigen display that could provide protection against SARS-CoV-2 after a single immunization.

In one embodiment, a nanoparticle is provided that displays a coronavirus spike protein or a portion thereof on its surface, wherein the nanoparticle comprises a fusion polypeptide comprising the coronavirus spike protein or an antigenic portion thereof linked to a first portion of a fibronectin binding protein comprising an aspartic acid residue that is covalently linked to a lysine residue in a second portion of the fibronectin binding protein via an isopeptide bond.

In one embodiment, a virus like particle (VLP) is provided that displays a coronavirus spike protein or a portion thereof, wherein the VLP comprises a coat protein of Fiersviridae comprising a first biotinylated, which first peptide is bound to streptavidin, e.g., divalent, trivalent or tetravalent (or more), that is bound to a second biotin that is bound to a second biotinylated peptide that is linked to the spike protein or a portion thereof that is also linked to trimerization domain, e.g., a T4 fibritin trimerization domain, a collagen trimerization domain, such as a collagen XV or XVIII trimerization domain, an influenza HA trimerization domain, a GCN4 trimerization domain, RSV F protein trimerization domain, HIV gp120 trimerization domain, or a EML2 trimerization domain..

In one embodiment, a nucleic acid vector is provided encoding a fusion polypeptide comprising a first coat protein of a Fiersviridae and a second coat protein of a Fiersviridae comprising a first peptide that is capable of being biotinylated.

In one embodiment, a nucleic acid vector is provided encoding a fusion polypeptide comprising a coronavirus spike protein or portion thereof, a peptide capable of being biotinylated, and a trimerization domain.

In one embodiment, isolated fusion polypeptide is provided comprising a first coat protein of a Fiersviridae and a second coat protein of a Fiersviridae comprising a first biotinylated peptide.

In one embodiment, isolated fusion polypeptide is provided comprising a coronavirus spike protein or portion thereof, a biotinylated peptide, and a trimerization domain.

In one embodiment, the present disclosure provides for a general platform for nanoparticle-based antigen display that could provide protection after a single immunization.

In one embodiment, a nanoparticle is provided that displays an influenza virus hemagglutinin (HA) protein, e.g., influenza A or influenza B HA, or a portion thereof on its surface, wherein the nanoparticle comprises a fusion polypeptide comprising the HA protein or an antigenic portion thereof linked to a first portion of a fibronectin binding protein comprising an aspartic acid residue that is covalently linked to a lysine residue in a second portion of the fibronectin binding protein via an isopeptide bond.

In one embodiment, a virus like particle (VLP) is provided that displays a HA protein or a portion thereof, wherein the VLP comprises a coat protein of Fiersviridae comprising a first biotinylated, which first peptide is bound to streptavidin, e.g., divalent, trivalent or tetravalent (or more), that is bound to a second biotin that is bound to a second biotinylated peptide that is linked to the HA protein or a portion thereof that is also optionally linked to trimerization domain, e.g., a T4 fibritin trimerization domain, a collagen trimerization domain, such as a collagen XV or XVIII trimerization domain, a heterologous influenza HA trimerization domain, a GCN4 trimerization domain, RSV F protein trimerization domain, HIV gp120 trimerization domain, or a EML2 trimerization domain..

In one embodiment, a nucleic acid vector is provided encoding a fusion polypeptide comprising a first coat protein of a Fiersviridae and a second coat protein of a Fiersviridae comprising a first peptide that is capable of being biotinylated.

In one embodiment, a nucleic acid vector is provided encoding a fusion polypeptide comprising a HA protein or portion thereof, a peptide capable of being biotinylated, and a trimerization domain.

In one embodiment, isolated fusion polypeptide is provided comprising a first coat protein of a Fiersviridae and a second coat protein of a Fiersviridae comprising a first biotinylated peptide.

In one embodiment, isolated fusion polypeptide is provided comprising a HA protein or portion thereof, a biotinylated peptide, and a trimerization domain.

In one embodiment, the present disclosure provides for a general platform for nanoparticle-based HA antigen, e.g., influenza A HA or influenza B HA, display that could provide protection after a single immunization.

Further provided in the present disclosure is a general platform for nanoparticle-based antigen display that could provide protection against a virus, bacteria or fungus, e.g., a microbial pathogen, after a single immunization.

In one embodiment, a nanoparticle is provided that displays an immunogenic protein of a microbial pathogen, such as a glycoprotein of a virus, a bacterial protein or a fungal protein, or a portion thereof on its surface, wherein the nanoparticle comprises a fusion polypeptide comprising the immunogenic microbial pathogen protein or an antigenic portion thereof, e.g., a glycoprotein, linked to a first portion of a fibronectin binding protein comprising an aspartic acid residue that is covalently linked to a lysine residue in a second portion of the fibronectin binding protein via an isopeptide bond.

In one embodiment, a virus like particle (VLP) is provided that displays an immunogenic protein of a microbial pathogen or a portion thereof, wherein the VLP comprises a coat protein of Fiersviridae comprising a first biotinylated, which first peptide is bound to streptavidin, e.g., divalent, trivalent or tetravalent (or more), that is bound to a second biotin that is bound to a second biotinylated peptide that is linked to the immunogenic protein of a microbial pathogen or a portion thereof, that is also linked to trimerization domain, e.g., a T4 fibritin trimerization domain, a collagen trimerization domain, such as a collagen XV or XVIII trimerization domain, an influenza HA trimerization domain, a GCN4 trimerization domain. RSV F protein trimerization domain, HIV gp120 trimerization domain, or a EML2 trimerization domain..

In one embodiment, isolated fusion polypeptide is provided comprising an immunogenic protein of a microbial pathogen or portion thereof, a biotinylated peptide, and a trimerization domain.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIGS. 1A-1C. Structure of the SARS-CoV-2 S ectodomain. A. The S1 and S2 domains are colored white and gray, respectively. B. Residues conserved between SARS-CoV-2 and SARS-CoV are shown in red. C. Residues conserved between SARS-CoV-2 and MERS-CoV are shown in magenta.

FIGS. 1D-1G. Assembly of VLP-S and characterization of MS2-SA VLP. D) Scheme illustrating assembly of VLP-S, where biotinylated MS2 (yellow, PDB: 2MS2) is added to streptavidin (red, PDB:3RY2) to create the VLP. S (green, PDB: 6VSB) biotinylated at the C-terminus is mixed with the VLP to create VLP-S. Biotinylated residues are colored blue. E) Size exclusion chromatography trace for MS2-SA VLP. The column void volume is 7.2 mL F) Characterization of the MS2-SA VLP by dynamic light scattering. G) Negative-stain transmission electron micrograph of MS2-SA VLPs.

FIGS. 2A-2E. Characterization of S2Pro and VLP-S2Pro. A) SDS-PAGE characterization of S_{2Pro} and VLP-S_{2Pro}. The VLP-S_{2Pro} has been boiled to disrupt the streptavidin-biotin conjugation. The unprocessed gel is shown in FIG. 7B) Size exclusion chromatography traces for S_{2Pro} (dashed line) and VLP-S_{2Pro} (solid line). The vertical gray line represents the peak elution volume of the molecular weight standard thyroglobulin (660 kDa). The column void volume is 7.2 mL. C) Characterization of the VLP-S_{2Pro} (solid line) by dynamic light scattering. D) Negative-stain transmission electron micrographs of S_{2Pro} incorporated on the surface of MS2-SA VLPs. Arrowheads (white) indicate the S_{2Pro} proteins on the VLP surface. E) Characterization of the binding of Fc-ACE2 (gray) and CR3022 (white) to S_{2Pro} and VLP-S_{2Pro} by ELISA (mean ± SD, n = 6: two independent assays, each with three technical replicates).

FIGS. 3A-3F. Characterization of S6Pro and VLP-S6Pro A) SDS-PAGE characterization of S₆ₚᵣₒ and VLP-S_{6Pro.} The VLP-S_{6Pro} has been boiled to disrupt the streptavidin-biotin conjugation. The unprocessed gel is shown in FIG. 7. B) Size exclusion chromatography traces for S_{6Pro} (dashed line) and VLP-S_{6Pro} (solid line). The vertical gray line represents the peak elution volume of the molecular weight standard thyroglobulin (660 kDa). The column void volume is 7.2 mL. C) Characterization of the VLP-S_{6Pro} (solid line) by dynamic light scattenng. D) Negative-stain transmission electron micrographs of S_{6Pro} incorporated on the surface of MS2-SA VLPs. Arrowheads (white) indicate the S_{6Pro} proteins on the VLP surface. E) Cryo-EM of vitrified VLP-S_{6Pro}. The inset shows a low-pass filtered to 10 Å volume of HexaPro structure (EMD: 22221, reported previously) for comparison. Arrowheads (black) indicate the representative S_{6Pro} proteins on the VLP surface. F) Characterization of the binding of Fc-ACE2 (gray) and CR3022 (white) to S_{6Pro} and VLP-S_{6Pro} by ELISA (mean + SD, n=6: two independent assays, each with three technical replicates).

FIGS. 4A-4D. Protective efficacy of VLP-S. A) Schedule for vaccination of hamsters, serum collection, infection, and organ collection. B) Body weight of hamsters immunized with a single dose of either VLP-S_{6Pro} (solid line with circles), VLP-S_{2Pro} (solid line with squares), MS2-SA VLP (dashed line with triangles), or PBS (dashed line with diamonds) after SARS-CoV-2 infection (mean ± SD, n=3 hamsters). ns: not statistically significant, **p < 0.01, determined by a one-way analysis of variance (ANOVA) and Dunnett post-hoc multiple comparison between groups {α = 0.1). Assumptions of the normality of residuals and homogeneity of variance were validated by the D′Agostino-Pearson test and the Brown-Forsythe test, respectively, C) Viral titer in the lungs of hamsters immunized with either PBS, MS2-SA VLP, VLP-S_{2Pro} or VLP-S_{6Pro} three days after SARS-CoV-2 infection (geometnc mean with geometric SD, n=3 hamsters). †-No infectious virus was detected in the lungs of hamsters immunized with VLP-S_{2Pro} or VLP-S_{6Pro} (detection limit 10 PFU/g). ns: not statistically significant, ****p < 0.0001, determined by a one-way analysis of variance (ANOVA) and Dunnett post-hoc multiple comparison between groups (α = 0.1). Assumptions of the normality of residuals and homogeneity of variance were validated by the Shapiro-Wilk test and the Brown-Forsythe test, respectively. D) Viral titer in the nasal turbinates of hamsters immunized with either PBS, MS2-SA VLP, VLP,S_{2Pro} or VLP-S_{6Pro} three days after SARS-CoV-2 infection (geometric mean with geometric SD, n = 3 hamsters). ns: not statistically significant, *p < 0.1, determined by a one-way analysis of variance (ANOVA) and Dunnett post-hoc multiple comparison between groups (α =0.1). Assumptions of the normality of residuals and homogeneity of variance were validated by the Shapiro-Wilk test and the Brown-Forsythe test, respectively.

FIGS. 5A-5B Characterization of MS2 to SA to S stoichiometry using SDS-PAGE. A) Amount of MS2 in heated MS2-SA VLP was compared to amount of MS2 in unheated MS2-SA VLP to determine that approximately 80 percent of MS2 is occupied by SA. Excess biotin was added to the unheated sample to occupy all unoccupied biotin binding sites prior to the addition of SDS. β-mercaptoethanol was added to all samples. B) Amount of unbound MS2-SA in VLP-S was compared to MS2-SA VLP standards to determine approximately 25 percent of MS2-SA in VLP-S was occupied by S. A heated control was included to ensure the same amount of VLP was present within the VLP-S and 100% VLP standard. Excess biotin was added to the unheated samples to occupy all unoccupied biotin binding sites prior to the addition of SDS. β-mercaptoethanol was added to all samples. The unprocessed gels are shown in FIG. 7.

FIGS. 6A-6B. SDS-PAGE gels of deglycosylated S proteins. S2Pro (A) and S6Pro (B) before and after deglycosylation with PNGase F. The unprocessed gel is shown in FIG. 7.

FIGS. 7A-7D. Unprocessed SDS-PAGE gel images. Unprocessed SDS-PAGE gel images, cropped versions of which appear in (A) FIG. 2a (solid rectangle), FIG. 3a (dashed rectangle), (B) FIG. 5a, (C) FIG. 5b, (D) FIG. 6a (solid rectangle), and FIG. 6b (dashed rectangle).

FIGS. 8A-8F. Assembly of VLP-S2 and characterization of MS2-SA VLP. A) The SARS-CoV-2 spike ectodomain (PDB: 6XKL). The S1 subunit is highlighted in orange and the S2 subunit is highlighted in green. B) Scheme illustrating the assembly of VLP-S2, where biotinylated MS2 (yellow, PDB: 2MS2) is added to streptavidin to create the VLP. S2 biotinylated at the C-terminus (green; PDB: 6XKL) is mixed with the VLP to create the VLP-S2. C) Size exclusion chromatography trace for MS2-SA VLP. The vertical gray line represents the peak elution volume of the molecular weight standard thyroglobulin (660 kDa). The column void volume is 7.2 mL. D) Characterization of the MS2-SA VLP by dynamic light scattering. E) Negative-stain transmission electron micrograph of MS2-SA VLPs. Scale bar = 50 nm. F) Cryo-EM of vitrified MS2-SA VLP. Scale bar = 50 nm.

FIGS. 9A-9F. Characterization of S2 and VLP-S2. A) SDS-PAGE characterization of S2 and VLP-S2. S2 was deglycosylated with PNGase F. The samples were heated with β-mercaptoethanol and LDS sample buffer. The unprocessed gel is shown in FIG. 12a. B) Size exclusion chromatography traces for S2 (dashed line) and VLP-S2 (solid line). The vertical gray line represents the peak elution volume of the molecular weight standard thyroglobulin (660 kDa). The column void volume is 7.2 mL. C) Characterization of the VLP-S2 by dynamic light scattering. D) Negative-stain transmission electron micrograph of VLP-S2. Arrowheads (white) indicate the S2 protein on the VLP surface. Scale bars = 50 nm. E) Cryo-1 EM of vitrified VLP-S2. Arrowheads (white) indicate the S2 protein on the VLP surface. Scale bars = 50 nm. F) Characterization of the binding of anti-S2 antibody 0304-3H3 to S2 and VLP-S2 by ELISA. (mean ± SD, n=6: two independent assays, each with three technical replicates).

FIGS. 10A-10F. Characterization of S2mutS2′ and VLP-S2mutS2′. A) SDS-PAGE characterization of 1 S2_{mutS2′} and VLP2 S2_{mutS2′}. S2_{mutS2′} was deglycosylated with PNGase F. The samples were heated with β-mercaptoethanol and LDS sample buffer. The unprocessed gel is shown in FIG. 12a. B) Size exclusion chromatography traces for S2_{mutS2′} (dashed line) and VLP-S2_{mutS2′} (solid line). The vertical gray line represents the peak elution volume of the molecular weight standard thyroglobulin (660 kDa). The column void volume is 7.2 mL. C) Characterization of the VLP-S2mutS2′ by dynamic light scattering. D) Negative-stain transmission electron micrograph of VLP-S2_{mutS2′}. Arrowheads (white) indicate the S2_{mutS2′} protein on the VLP surface. Scale bars = 50 nm. E) Cryo-EM of vitrified VLP-S2_{mutS2′}. Arrowheads (white) indicate the S2_{mutS2′} protein on the VLP surface. Scale bars = 50 nm. F) Characterization of the binding of anti-S2 antibody 0304-3H3 to S2 and VLP-S2 by ELISA. (mean ± SD, n=6: two independent assays, each with three technical replicates).

FIGS. 11A-11E. Protective efficacy of VLP-S2 and VLP-S2_{mutS2′}. A) Schedule for hamster 1 vaccination, serum collection, infection with SARS-CoV-2, and organ collection. B) Antibody endpoint titers of sera from hamsters immunized with either VLP-S2, VLP-S2_{mutS2}, or MS2-SA VLP against SARS-CoV-2 spike protein (geometric mean with geometric SD, n=6: two independent assays with sera from 3 hamsters). ns: not statistically significant, ****p < 0.0001, determined by a one-way analysis of variance (ANOVA) and Tukey post-hoc multiple comparison between groups (α = 0.05). C) Viral titer in the lungs of hamsters immunized with either 7 VLP-S2, VLP-S2_{mutS2′}, or MS2-SA VLP three days after infection with SARS-CoV-2 (geometric mean with geometric SD, n=3 hamsters). **p < 0.01, determined by a one-way analysis of variance (ANOVA) and Dunnett post-hoc multiple comparison between groups (α = 0.05). D) Viral titer in the nasal turbinates of hamsters immunized with either MS2-SA VLP, VLP-S2, or VLP-S2mutS2 three days after SARS-CoV-2 infection (geometric mean with geometric SD, n=3 hamsters). **p < 0.01, determined by a one-way analysis of variance (ANOVA) and Dunnett post-hoc multiple comparison between groups (α = 0.05). E) Antibody endpoint titers of sera from hamsters immunized with either VLP-S2 (gray), VLP-S2_{mutS2} (white), or MS2-SA VLP against spike proteins of the original Wuhan-Hu-1 SARS-CoV-2 (614D), the SARS-CoV-2 variant B.1.351, SARS-CoV-1, and the four endemic human coronaviruses HKU-1, OC43, NL63, and 229E (geometric mean with geometric SD, n=6 against SARS-CoV-2 614D S protein: two independent assays with sera from 3 hamsters; n=3 against all other S proteins: sera from 3 hamsters). ns: not statistically significant, ****p < 0.0001, determined by a one way analysis of variance (ANOVA) and Tukey post-hoc multiple comparison between groups (α = 0.05).

FIGS. 12A-12B. Characterization of MS2 to SA to S stoichiometry using SDS-PAGE. A) Amount of MS2 in heated MS2-SA VLP was compared to amount of MS2 in unheated MS2-SA VLP to determine that approximately 78 percent of MS2 was bound to SA. Excess biotin was added to the unheated sample to occupy all unoccupied biotin binding sites prior to the addition of SDS. β-mercaptoethanol was added to all samples. B) The intensity of bands corresponding to S2 and MS2 in VLP-S2 and VLP-S2mutS2′ were compared to BSA standards and quantified to determine that approximately 30 S2 molecules were displayed on each MS2-SA VLP. S2 was deglycosylated with PNGase F and all samples were heated with β-mercaptoethanol and LDS sample buffer. The unprocessed gels are shown in FIG. 13.

FIGS. 13A-13C. Unprocessed SDS-PAGE gel images. Unprocessed SDS-PAGE gel images, cropped versions of which appear in (A) FIG. 9a (solid rectangle), FIG. 10a (dashed rectangle), (B) FIG. 12a, and (C) FIG. 12b.

FIG. 14. Schematic illustrating the generation of RBD-SpyCatcher-mi3 conjugates by the reaction of RBD-SpyTag with Spycatcher-mi3 nanoparticles. (RBD: magenta; SpyTag: green; SpyCatcher: yellow; mi3: cyan).

FIGS. 15A-15D. Characterization of RBD and RBD-SpyCatcher-mi3. A) Characterization of SpyCatcher-mi3, RBD, and RBD-SpyCatcher-mi3 by SDS-PAGE. The unprocessed gel is shown in FIG. 18. B) Size exclusion chromatography curves for RBD (dashed line) and RBD-SpyCatcher-mi3 (solid line). The gray line represents the peak elution volume of the molecular weight standard thyroglobulin. The column void volume is 7.2 mL.C). Characterization of the RBD-SpyCatcher-mi3 (solid line) and SpyCatcher-mi3 (dashed line) by dynamic light scattering. D) Characterization of the binding of ACE-2-Fc (dark gray), CR3022 (light gray), and S309 (white) to RBD, RBD-SpyCatcher-mi3, and BSA (control) by ELISA (mean ± SD, n=6: two assays with three technical replicates).

FIGS. 16A-16B. Antibody Response to RBD-SpyCatcher-mi3. A) Antibody endpoint titers of sera from mice immunized with a prime and boost of RBD-SpyCatcher-mi3 against the spike proteins of an early isolate of SARS-CoV-2 (S-614D), SARS-CoV-2 variants B.1.1.7, B.1.351, and P.1, and SARS-CoV-1 (geometric mean with geometric SD, n = 3 against all S protein: sera from 3 mice). ns = not statistically significant, determined by a one-way analysis of variance (ANOVA) and Tukey post-hoc multiple comparison between groups (α = 0.05). B) Viral neutralization titers for sera from mice immunized with RBD-SpyCatcher-mi3 (geometric mean with geometric SD, n = 6 against S-614D and n = 3 against all other viral strains: sera from 3 mice). Endpoint titers using 2-fold diluted sera were expressed as the reciprocal of the highest dilution that completely prevented cytopathic effects. ns = not statistically significant, determined by a one-way analysis of variance (ANOVA) and Tukey post-hoc multiple comparison between groups (α = 0.05).

FIG. 17. Characterization of RBD and mi3 conjugation stoichiometry by SDS-PAGE. Amount of unbound SpyCatcher-mi3 monomer was compared to SpyCatcher-mi3 standards (left side) to determine coverage of RBD on SpyCatcher-mi3. ~50% of SpyCatcher-mi3 monomers reacted, indicating each particle contained ~30 RBD proteins.

FIG. 18. Antibody endpoint titers of sera from mice immunized with a single dose of RBDSpyCatcher-mi3 against the spike proteins of an early isolate of SARS-CoV-2 (S-614D) and SARS-CoV-2 variants B.1.1.7, B.1.351, and P.1 (geometric mean with geometric SD, n = 3 against all S protein: sera from 3 mice). ns = not statistically significant, determined by a one-way analysis of variance (ANOVA) and Tukey post-hoc multiple comparison between groups (α = 0.05).

FIGS. 19A-19B. Unprocessed SDS-PAGE gel images. Cropped versions appear in (A) FIG. 15a and (B) FIG. 17.

FIGS. 20A-20C. VLP-based scaffolds presenting multiple copies of HA. A) Representation of VLP-HA conjugates. B) VLP-HA conjugates elicit significantly higher titers of neutralizing antibodies than HA trimers after a single immunization (three weeks post-prime). C) Characterization of neutralizing antibody titers elicited by VLP-HA with either AddaVax or Quil-A adjuvant 3 to 40 weeks post-prime.

FIGS. 21A-21B. Neutralizing antibodies elicited by immunization with VLP-HA conjugates in ferrets. (A) Timeline of ferret immunization and serial blood draw. (B) Ferrets were subcutaneously immunized with VLP-HA containing 45 µg of PR8 HA, adjuvanted with either AddaVax (500 µL), Quil-A (30 µg), or poly I:C (3 µg). At 3, 8, 20, 40, and 60 weeks post immunization, the animals were bled and the serum neutralizing antibody titers against the homologous virus A/Puerto Rico/8/34 (PR8) were examined.

FIGS. 22A-22B. Neutralizing antibodies elicited by immunization with recombinant HA protein in ferrets. (A) Timeline of ferret immunization and blood draw. (B) Ferrets were intramuscularly mock-immunized (No vac.) or immunized with recombinant HA protein (15 µg) (HA unconjugated to VLP) of A/Netherlands/312/2003 virus, adjuvanted with either AddaVax (100 µL), Quil-A (30 µg), or Alhydrogel adjuvant 2% (100 µL). At 3 weeks post immunization, the animals were bled and the serum neutralizing antibody titers against the homologous virus A/Netherlands/312/2003 virus were examined.

FIG. 23. Neutralizing antibodies elicited by reduced amounts of VLP-HA antigen in ferrets. Ferrets were immunized with the indicated amount of VLP-HA antigen containing PR8 HA adjuvanted with AddaVax, either via subcutaneous (s.c.) or intramuscular (i.m.) route. At 3 weeks post immunization, the ferrets were bled to analyze serum neutralization titers against the homologous virus PR8. Dots show the data of individual animals.

FIG. 24. Exemplary HA sequences.

FIG. 25. Exemplary HA sequences.

FIGS. 26A-26F. Assembly of VLP-S2 and characterization of MS2-SA VLP. (A) The SARS-CoV-2 spike ectodomain (PDB: 6XKL). The S1 subunit is highlighted in orange and the S2 subunit is highlighted in green. (B) Scheme illustrating the assembly of VLP-S2, where biotinylated MS2 (yellow, PDB: 2MS2) is added to streptavidin to create the VLP. S2 biotinylated at the C-terminus (green; PDB: 6XKL) is mixed with the VLP to create the VLP-S2. (C) Size exclusion chromatography trace for MS2-SA VLP. The vertical gray line represents the peak elution volume of the molecular weight standard thyroglobulin (660 kDa). The column void volume is 7.2 mL. (D) Characterization of the MS2-SA VLP by dynamic light scattering. (E) Negative-stain transmission electron micrograph of MS2-SA VLPs. Scale bar = 50 nm. (F) Cryo-EM of vitrified MS2-SA VLP. Scale bar = 50 nm.

FIGS. 27A-27F. Characterization of S2 and VLP-S2. (A) SDS-PAGE characterization of S2 and VLP-S2. S2 was deglycosylated with PNGase F. The samples were heated with β-mercaptoethanol and LDS sample buffer. The unprocessed gel is shown in Fig. S2a. (B) Size exclusion chromatography traces for S2 (dashed line) and VLP-S2 (solid line). The vertical gray line represents the peak elution volume of the molecular weight standard thyroglobulin (660 kDa). The column void volume is 7.2 mL. (C) Characterization of the VLP-S2 by dynamic light scattering. (D) Negative-stain transmission electron micrograph of VLP-S2. Arrowheads (white) indicate the S2 protein on the VLP surface. Scale bars = 50 nm. (E) Cryo-EM of vitrified VLP-S2. Arrowheads (white) indicate the S2 protein on the VLP surface. Scale bars = 50 nm. (F) Characterization of the binding of anti-S2 antibody 0304-3H3 to S2 and VLP-S2 by ELISA. (mean ± SD, n=6: two independent assays, 1 each with three technical replicates).

FIGS. 28A-28F. Characterization of S2_{mutS2′} and VLP-S2_{mutS2′}. (A) SDS-PAGE characterization of S2_{mutS2′} and VLP-S2_{mutS2′}. S2_{mutS2′} was deglycosylated with PNGase F. The samples were heated with β-mercaptoethanol and LDS sample buffer. The unprocessed gel is shown in Fig. S2a. (B) Size exclusion chromatography traces for S2_{mutS2′} (dashed line) and VLP-S2_{mutS2′} (solid line). The vertical gray line represents the peak elution volume of the molecular weight standard thyroglobulin (660 kDa). The column void volume is 7.2 mL. (C) Characterization of the VLP-S2_{mutS2′} by dynamic light scattering. (D) Negative-stain transmission electron micrograph of VLP-S2_{mutS2′}. Arrowheads 1 (white) indicate the S2_{mutS2′} protein on the VLP surface. Scale bars = 50 nm. (E) Cryo-EM of vitrified VLP-S2_{mutS2′}. Arrowheads (white) indicate the S2_{mutS2′} protein on the VLP surface. Scale bars = 50 nm. (F) Characterization of the binding of anti-S2 antibody 0304-3H3 to S2 and VLP-S2 by ELISA. (mean ± SD, n=6: two independent assays, each with three technical replicates).

FIGS. 29A-29E. Protective efficacy of VLP-S2 and VLP-S2_{mutS2′}. (A) Schedule for hamster vaccination, serum collection, infection with SARS-CoV-2, and organ collection. (B) Antibody endpoint titers of sera from hamsters immunized with either VLP-S2, VLP-S2_{mutS2}, or VLP-control against SARS-4 CoV-2 spike protein (geometric mean with geometric SD, n=6: two independent assays with sera from 3 hamsters). ns: not statistically significant, ****p < 0.0001, determined by a one-way analysis of variance (ANOVA) and Tukey post-hoc multiple comparison between groups (α = 0.05). (C) Viral titer in the lungs of hamsters immunized with either VLP-S2, or VLP-S2_{mutS2′}, or VLP-control three days after infection with SARS-CoV-2 (mean with SD, n=3 hamsters). **p < 0.01, determined by a one-way analysis of variance (ANOVA) and Dunnett post-hoc multiple comparison between groups (α = 0.05). (D) Viral titer in the nasal turbinates of hamsters immunized with either VLP-S2, or VLP-_{S2mutS2}, or VLP-control three days after SARS-CoV-2 infection (mean with SD, n=3 7 hamsters). **p < 0.01, determined by a one-way analysis of variance (ANOVA) and Dunnett post-hoc multiple comparison between groups (α = 0.05). (E) Antibody endpoint titers of sera from hamsters immunized with either VLP-control, VLP-S2 (gray), or VLP-S2_{mutS2} (white) against spike proteins of the original Wuhan-Hu-SARS-CoV-2 (614D), the SARS-CoV-2 variant B.1.351, SARS-CoV-1, and the four endemic human coronaviruses HKU-1, OC43, NL63, and 229E (geometric mean with geometric SD, n=6 against SARS-CoV-2 614D S protein: two independent assays with sera from 3 hamsters; n=3 against all other S proteins: sera from 3 hamsters). ns: not statistically significant, ****p < 0.0001, determined by a one-way analysis of variance (ANOVA) and Tukey post-hoc multiple comparison between groups (α = 0.05).

FIGS. 30A-30D. Increasing the protective efficacy of VLP-S2_{mutS2′}. (A) Viral titers in the lungs (left) and nasal turbinates (right) of hamsters immunized with two doses of either VLP-S2_{mutS2′} or VLP-control adjuvanted with AddaVax, three days after infection with B.1.617.2 (mean with SD, n = 4: sera from four hamsters). ns: not statistically significant, determined by two-tailed Welch’s t-test. (B) Viral titers in the lungs (left) and nasal turbinates (right) of hamsters immunized with three doses of either VLP-S2_{mutS2′} or VLP-control adjuvanted with AddaVax, three days after infection with B.1.617.2 (mean with SD, n = 3: sera from three hamsters). ***p < 0.001, determined by two-tailed unpaired t-test. (C) Viral titers in the lungs of hamsters immunized with two doses of either VLP-S2_{mutS2′} or VLP-control mixed with various adjuvants, three days after infection with B.1.617.2 (mean with SD, n = 3: sera from three hamsters). ns: not statistically significant, **p < 0.01, ***p < 0.001 determined by two-tailed unpaired t-test. (D) Viral titers in the nasal turbinates of hamsters immunized with two doses of either VLP-S2_{mutS2′} or VLP-control mixed with various adjuvants, three days after infection with B.1.617.2 (mean with SD, n = 3: sera from three hamsters). ns: not statistically significant, ***p < 0.001 determined by two-tailed unpaired t-test.

FIGS. 31A-31E. Evaluating the breadth of the protective efficacy of the immunization regimen via a challenge with SARS-CoV-2 variants of concern and pangolin coronaviruses. (A) Viral antibody endpoint titers against S proteins from various coronaviruses (geometric mean with geometric SD, n = 14: sera from 14 hamsters) ns: not statistically significant, *p < 0.05, ****p < 0.0001, determined by Brown-Forsythe and Welch ANOVA tests. (B) Viral titers in the lungs of hamsters immunized with either VLP-S2_{mutS2′} or VLP-control adjuvanted with AS03 + pIC, three days after infection with SARS-CoV-2 variants (mean with SD, n = 4 for BA.1: sera from four hamsters, n = 3 for all other coronaviruses: sera from three hamsters). *p < 0.05, **p < 0.01, ****p < 0.0001, determined by two-tailed Welch’s t-test. (C) Viral titers in the nasal turbinates of hamsters immunized with either VLP-S2_{mutS2′} or VLP-control adjuvanted with AS03 + pIC, three days after infection with SARS-CoV-2 variants (mean with SD, n = for BA.1: sera from four hamsters, n = 8 for all other S proteins: sera from three hamsters). ns: not statistically significant, *p < 0.05, **p < 0.01 determined by two-tailed Welch’s t-test. (D) Viral titers in the lungs (left) and nasal turbinates (right) of hamsters immunized with either VLP-S2_{mutS2′} or VLP-control adjuvanted with AS03 + pIC, three days after infection with Pg-CoV (mean with SD, n = 4: sera from four hamsters) **p < 0.01, 12 ***p < 0.001, determined by two-tailed Welch’s t-test. † – No infectious virus was detected in the lungs of immunized hamsters. Detection limit (dotted line) = 1.3 log₁₀ pfu/g. (E) Percent neutralization against SARS-CoV-2 early isolate S-614G after immunization with VLP-S2_{mutS2′}. FRNT₅₀ = 34.7 (mean with SD, n = 14 for VVLP-S2_{mutS2′}: sera from hamsters).

FIGS. 32A-32B. Characterization of MS2 to SA to S stoichiometry using SDS-PAGE. (A) Amount of MS2 in heated MS2-SA VLP was compared to amount of MS2 in unheated MS2-SA VLP to determine that approximately 78 percent of MS2 was bound to SA. Excess biotin was added to the unheated sample to occupy all unoccupied biotin binding sites prior to the addition of SDS. β-mercaptoethanol was added to all samples. (B) The intensity of bands corresponding to S2 and MS2 in VLP-S2 and VLP-S2_{mutS2′} were compared to BSA standards and quantified to determine that approximately 30 S2 molecules were displayed on each MS2-SA VLP. S2 was deglycosylated with PNGase F and all samples were heated with β-mercaptoethanol and LDS sample buffer.

FIGS. 33A-33B. Neutralizing antibodies elicited by immunization with VLP-HA conjugates in ferrets. (A) Timeline of ferret immunization and serial blood draw. (B) Ferrets were subcutaneously immunized with VLP-HA containing 45 µg of PR8 HA, adjuvanted with either AddaVax (500 µL), Quil-A (30 µg), or poly I:C (3 µg). At 3, 8, 20, 40, 60, 100, and 120 weeks post immunization, the animals were bled and the serum neutralizing antibody titers against the homologous virus A/Puerto Rico/8/34 (PR8) were examined.

FIGS. 34A-34B. Neutralizing antibodies elicited by immunization with recombinant HA protein in ferrets. (A) Timeline of ferret immunization and bloo draw. (B) Ferrets were intramuscularly mock-immunized (No vac.) or immunized with recombinant HA protein (15 µg) of A/Netherlands/312/2003 virus, adjuvanted with either AddaVax (100 µL), Quil-A (30 µg), or Alhydrogel adjuvant 2% (100 µL). At 3 weeks post immunization, the animals were bled and the serum neutralizing antibody titers against the homologous virus A/Netherlands/312/2003 virus were examined.

FIG. 35. Neutralizing antibodies elicited by reduced amounts of VLP-HA antigen in ferrets. Ferrets were immunized with the indicated amount of VLP-HA antigen containing PR8 HA adjuvanted with AddaVax, either via subcutaneous (s.c.) or intramuscular (i.m.) route. At 3 weeks post immunization, the ferrets were bled to analyze serum neutralization titers against the homologous virus PR8. Dots show the data of individual animals.

FIGS. 36A-36B. Evaluating the efficacy of VLP-S2_{mutS2′} in a mouse model. (A) Viral titers in the lungs of mice immunized with one dose of either VLP-S2_{mutS2′} or VLP-control adjuvanted with AS03 + pIC, three days after infection with mouse-adapted SARS-CoV-2 strain, MA10. (mean with SD. biological replicates and n =5 for VLP-S2_{mutS2′:} tissues from five mice, biological replicates and n =7 for VLP-control: tissues from seven mice). ****P< 0.0001 [two-tailed Welch’s t-test]. † – No infectious virus was detected in the lungs of immunized mice. Detection limit (dotted line) = 1.3 log₁₀ pfu/g. (B) Percent neutralization against SARS-CoV-2 early isolate S-614G, B.1.617.2, BA.1. and Pg-CoV after immunization with one dose of VLP-S2_{mutS2}. FRNT50 =247 for S-614G, 181 for B.1.617.2, <20 for BA.1, and 288 for Pg-CoV (mean with SD, biological replicates and n =3 for BA.1: sera from three mice, biological replicates and n = 4 for other coronaviruses: sera from four mice).

### DETAILED DESCRIPTION

### Definitions

A “vector” or “construct” (sometimes referred to as gene delivery or gene transfer “vehicle”) refers to a macromolecule or complex of molecules comprising a polynucleotide, virus or virus like particle (VLP) to be delivered to a host cell, either in vitro or in vivo. The polynucleotide, virus or VLP to be delivered may comprise a coding sequence of interest for gene therapy. Vectors include, for example, macromolecular complexes capable of mediating delivery of the complexes to a host cell. Vectors can also comprise other components or functionalities that further modulate gene delivery and/or gene expression, or that otherwise provide beneficial properties to the targeted cells. Such other components include, for example, components that influence binding or targeting to cells (including components that mediate cell-type or tissue-specific binding); components that influence uptake of the vector nucleic acid by the cell; components that influence localization of the polynucleotide within the cell after uptake (such as agents mediating nuclear localization); and components that influence expression of the polynucleotide. Such components also might include markers, such as detectable and/or selectable markers that can be used to detect or select for cells that have taken up and are expressing the nucleic acid delivered by the vector. Such components can be provided as a natural feature of the vector (such as the use of certain viral vectors which have components or functionalities mediating binding and uptake), or vectors can be modified to provide such functionalities. A large variety of such vectors are known in the art and are generally available. When a vector is maintained in a host cell, the vector can either be stably replicated by the cells during mitosis as an autonomous structure, incorporated within the genome of the host cell, or maintained in the host cell’s nucleus or cytoplasm.

“Gene delivery,” “gene transfer,” and the like as used herein, are terms referring to the introduction of an exogenous polynucleotide (sometimes referred to as a “transgene”) into a host cell, irrespective of the method used for the introduction. Such methods include a variety of well-known techniques such as vector-mediated gene transfer (by, e.g., viral infection/transfection, or various other protein-based or lipid-based gene delivery complexes) as well as techniques facilitating the delivery of “naked” polynucleotides (such as electroporation, “gene gun” delivery and various other techniques used for the introduction of polynucleotides). The introduced polynucleotide may be stably or transiently maintained in the host cell. Stable maintenance typically requires that the introduced polynucleotide either contains an origin of replication compatible with the host cell or integrates into a replicon of the host cell such as an extrachromosomal replicon (e.g., a plasmid) or a nuclear or mitochondrial chromosome. A number of vectors are known to be capable of mediating transfer of genes to mammalian cells, as is known in the art.

By “transgene” is meant any piece of a nucleic acid molecule (for example, DNA) which is inserted by artifice into a cell either transiently or permanently, and becomes part of the organism if integrated into the genome or maintained extrachromosomally. Such a transgene may include at least a portion of an open reading frame of a gene which is partly or entirely heterologous (i.e., foreign) to the transgenic organism, or may represent at least a portion of an open reading frame of a gene homologous to an endogenous gene of the organism, which portion optionally encodes a polypeptide with substantially the same activity as the corresponding full-length polypeptide or at least one activity of the corresponding full-length polypeptide.

By “transgenic cell” is meant a cell containing a transgene. For example, a cell stably or transiently transformed with a vector containing an expression cassette is a transgenic cell that can be used to produce a population of cells having altered phenotypic characteristics. A “recombinant cell” is one which has been genetically modified, e.g., by insertion, deletion or replacement of sequences in a nonrecombinant cell by genetic engineering.

The term “wild-type” or “native” refers to a gene or gene product that has the characteristics of that gene or gene product when isolated from a naturally occurring source. A wild-type gene is that which is most frequently observed in a population and is thus arbitrarily designated the “normal” or “wild-type” form of the gene. In contrast, the term “modified” or “mutant” refers to a gene or gene product that displays modifications in sequence and or functional properties (i.e., altered characteristics) when compared to the wild-type gene or gene product. It is noted that naturally-occurring mutants can be isolated; these are identified by the fact that they have altered characteristics when compared to the wild-type gene or gene product.

The term “transduction” denotes the delivery of a polynucleotide to a recipient cell either in vivo or in vitro, via a viral vector and preferably via a replication-defective viral vector.

The term “heterologous” as it relates to nucleic acid sequences such as gene sequences encoding a protein and control sequences, denotes sequences that are not normally joined together, and/or are not normally associated with a particular cell, e.g., are from different sources (for instance, sequences from a virus are heterologous to sequences in the genome of an uninfected cell). Thus, a “heterologous” region of a nucleic acid construct or a vector is a segment of nucleic acid within or attached to another nucleic acid molecule that is not found in association with the other molecule in nature. For example, a heterologous region of a nucleic acid construct could include a coding sequence flanked by sequences not found in association with the coding sequence in nature, i.e., a heterologous promoter. Another example of a heterologous coding sequence is a construct where the coding sequence itself is not found in nature (e.g., synthetic sequences having codons different from the native gene). Similarly, a cell transformed with a construct which is not normally present in the cell would be considered heterologous for purposes of this invention.

By “DNA” is meant a polymeric form of deoxyribonucleotides (adenine, guanine, thymine, or cytosine) in double-stranded or single-stranded form found, inter alia, in linear DNA molecules (e.g., restriction fragments), viruses, plasmids, and chromosomes. In discussing the structure of particular DNA molecules, sequences may be described herein according to the normal convention of giving only the sequence in the 5′ to 3′ direction along the nontranscribed strand of DNA (i.e., the strand having the sequence complementary to the mRNA). The term captures molecules that include the four bases adenine, guanine, thymine, or cytosine, as well as molecules that include base analogues which are known in the art.

As used herein, the terms “complementary” or “complementarity” are used in reference to polynucleotides (i.e., a sequence of nucleotides) related by the base-pairing rules. For example, the sequence “A-G-T,” is complementary to the sequence “T-C-A.” Complementarity may be “partial,” in which only some of the nucleic acids’ bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids.

DNA molecules are said to have “5′ ends” and “3′ ends” because mononucleotides are reacted to make oligonucleotides or polynucleotides in a manner such that the 5′ phosphate of one mononucleotide pentose ring is attached to the 3′ oxygen of its neighbor in one direction via a phosphodiester linkage. Therefore, an end of an oligonucleotide or polynucleotide is referred to as the “5′ end” if its 5′ phosphate is not linked to the 3′ oxygen of a mononucleotide pentose ring and as the “3′ end” if its 3′ oxygen is not linked to a 5′ phosphate of a subsequent mononucleotide pentose ring. As used herein, a nucleic acid sequence, even if internal to a larger oligonucleotide or polynucleotide, also may be said to have 5′ and 3′ ends. In either a linear or circular DNA molecule, discrete elements are referred to as being “upstream” or 5′ of the “downstream” or 3′ elements. This terminology reflects the fact that transcription proceeds in a 5′ to 3′ fashion along the DNA strand. The promoter and enhancer elements that direct transcription of a linked gene are generally located 5′ or upstream of the coding region. However, enhancer elements can exert their effect even when located 3′ of the promoter element and the coding region. Transcription termination and polyadenylation signals are located 3′ or downstream of the coding region.

A “gene,” “polynucleotide,” “coding region,” “sequence,” “segment, ” “fragment” or “transgene” which “encodes” a particular protein, is a nucleic acid molecule which is transcribed and optionally also translated into a gene product, e.g., a polypeptide, in vitro or in vivo when placed under the control of appropriate regulatory sequences. The coding region may be present in either a cDNA, genomic DNA, or RNA form. When present in a DNA form, the nucleic acid molecule may be single-stranded (i.e., the sense strand) or double-stranded. The boundaries of a coding region are determined by a start codon at the 5′ (amino) terminus and a translation stop codon at the 3′ (carboxy) terminus. A gene can include, but is not limited to, cDNA from prokaryotic or eukaryotic mRNA, genomic DNA sequences from prokaryotic or eukaryotic DNA, and synthetic DNA sequences. A transcription termination sequence will usually be located 3′ to the gene sequence.

The term “control elements” refers collectively to promoter regions, polyadenylation signals, transcription termination sequences, upstream regulatory domains, origins of replication, internal ribosome entry sites (“IRES”), enhancers, splice junctions, and the like, which collectively provide for the replication, transcription, post-transcriptional processing and translation of a coding sequence in a recipient cell. Not all of these control elements need always be present so long as the selected coding sequence is capable of being replicated, transcribed and translated in an appropriate host cell.

The term “promoter” is used herein in its ordinary sense to refer to a nucleotide region comprising a DNA regulatory sequence, wherein the regulatory sequence is derived from a gene which is capable of binding RNA polymerase and initiating transcription of a downstream (3′ direction) coding sequence.

By “enhancer” is meant a nucleic acid sequence that, when positioned proximate to a promoter, confers increased transcription activity relative to the transcription activity resulting from the promoter in the absence of the enhancer domain.

By “operably linked” with reference to nucleic acid molecules is meant that two or more nucleic acid molecules (e.g., a nucleic acid molecule to be transcribed, a promoter, and an enhancer element) are connected in such a way as to permit transcription of the nucleic acid molecule. “Operably linked” with reference to peptide and/or polypeptide molecules is meant that two or more peptide and/or polypeptide molecules are connected in such a way as to yield a single polypeptide chain, i.e., a fusion polypeptide, having at least one property of each peptide and/or polypeptide component of the fusion. The fusion polypeptide is preferably chimeric, i.e., composed of heterologous molecules.

“Homology” refers to the percent of identity between two polynucleotides or two polypeptides. The correspondence between one sequence and to another can be determined by techniques known in the art. For example, homology can be determined by a direct comparison of the sequence information between two polypeptide molecules by aligning the sequence information and using readily available computer programs. Alternatively, homology can be determined by hybridization of polynucleotides under conditions which form stable duplexes between homologous regions, followed by digestion with single strand-specific nuclease(s), and size determination of the digested fragments. Two DNA, or two polypeptide, sequences are “substantially homologous” to each other when at least about 80%, preferably at least about 90%, and most preferably at least about 95% of the nucleotides, or amino acids, respectively match over a defined length of the molecules, as determined using the methods above.

By “mammal” is meant any member of the class Mammalia including, without limitation, humans and nonhuman primates such as chimpanzees and other apes and monkey species; farm animals such as cattle, sheep, pigs, goats and horses; domestic mammals such as dogs and cats; laboratory animals including rodents such as mice, rats, rabbits and guinea pigs, and the like.

By “derived from” is meant that a nucleic acid molecule was either made or designed from a parent nucleic acid molecule, the derivative retaining substantially the same functional features of the parent nucleic acid molecule, e.g., encoding a gene product with substantially the same activity as the gene product encoded by the parent nucleic acid molecule from which it was made or designed.

By “expression construct” or “expression cassette” is meant a nucleic acid molecule that is capable of directing transcription. An expression construct includes, at the least, a promoter. Additional elements, such as an enhancer, and/or a transcription termination signal, may also be included.

The term “exogenous,” when used in relation to a protein, gene, nucleic acid, or polynucleotide in a cell or organism refers to a protein, gene, nucleic acid, or polynucleotide which has been introduced into the cell or organism by artificial or natural means. An exogenous nucleic acid may be from a different organism or cell, or it may be one or more additional copies of a nucleic acid which occurs naturally within the organism or cell. By way of a non-limiting example, an exogenous nucleic acid is in a chromosomal location different from that of natural cells, or is otherwise flanked by a different nucleic acid sequence than that found in nature.

The term “isolated” when used in relation to a nucleic acid, peptide, polypeptide, virus or VLP refers to a nucleic acid sequence, peptide, polypeptide, virus or VLP that is identified and separated from at least one contaminant nucleic acid, polypeptide or other biological component with which it is ordinarily associated in its natural source, e.g., so that it is not associated with in vivo substances, or is substantially purified from in vitro substances. Isolated nucleic acid, peptide, polypeptide, VLP or virus is present in a form or setting that is different from that in which it is found in nature. For example, a given DNA sequence (e.g., a gene) is found on the host cell chromosome in proximity to neighboring genes; RNA sequences, such as a specific mRNA sequence encoding a specific protein, are found in the cell as a mixture with numerous other mRNAs that encode a multitude of proteins. The isolated nucleic acid molecule may be present in single-stranded or double-stranded form. When an isolated nucleic acid molecule is to be utilized to express a protein, the molecule will contain at a minimum the sense or coding strand (i.e., the molecule may single-stranded), but may contain both the sense and anti-sense strands (i.e., the molecule may be double-stranded).

As used herein, the term “recombinant nucleic acid” or “recombinant DNA sequence, molecule or segment” refers to a nucleic acid, e.g., to DNA, that has been derived or isolated from a source, that may be subsequently chemically altered in vitro, and includes, but is not limited to, a sequence that is naturally occurring, is not naturally occurring, or corresponds to naturally occurring sequences that are not positioned as they would be positioned in the native genome. An example of DNA “derived” from a source, would be a DNA sequence that is identified as a useful fragment, and which is then chemically synthesized in essentially pure form. An example of such DNA “isolated” from a source would be a useful DNA sequence that is excised or removed from said source by chemical means, e.g., by the use of restriction endonucleases, so that it can be further manipulated, e.g., amplified, for use in the invention, by the methodology of genetic engineering.

The term “recombinant protein” or “recombinant polypeptide” as used herein refers to a protein molecule that is expressed from a recombinant DNA molecule.

The term “peptide”, “polypeptide” and protein” are used interchangeably herein unless otherwise distinguished.

The term “sequence homology” means the proportion of base matches between two nucleic acid sequences or the proportion amino acid matches between two amino acid sequences. When sequence homology is expressed as a percentage, e.g., 50%, the percentage denotes the proportion of matches over the length of a selected sequence that is compared to some other sequence. Gaps (in either of the two sequences) are permitted to maximize matching; gap lengths of 15 bases or less are usually used, 6 bases or less are preferred with 2 bases or less more preferred. When using oligonucleotides as probes or treatments, the sequence homology between the target nucleic acid and the oligonucleotide sequence is generally not less than 17 target base matches out of 20 possible oligonucleotide base pair matches (85%); preferably not less than 9 matches out of 10 possible base pair matches (90%), and more preferably not less than 19 matches out of 20 possible base pair matches (95%).

Two amino acid sequences are homologous if there is a partial or complete identity between their sequences. For example, 85% homology means that 85% of the amino acids are identical when the two sequences are aligned for maximum matching. Gaps (in either of the two sequences being matched) are allowed in maximizing matching; gap lengths of 5 or less are preferred with 2 or less being more preferred. Alternatively and preferably, two protein sequences (or polypeptide sequences derived from them of at least 30 amino acids in length) are homologous, as this term is used herein, if they have an alignment score of at more than 5 (in standard deviation units) using the program ALIGN with the mutation data matrix and a gap penalty of 6 or greater. The two sequences or parts thereof are more preferably homologous if their amino acids are greater than or equal to 50% identical when optimally aligned using the ALIGN program.

The term “corresponds to” is used herein to mean that a polynucleotide sequence is homologous (e.g., is identical, not strictly evolutionarily related) to all or a portion of a reference polynucleotide sequence that encodes a polypeptide or its complement, or that a polypeptide sequence is identical in sequence or function to a reference polypeptide sequence. For illustration, the nucleotide sequence “TATAC” corresponds to a reference sequence “TATAC” and is complementary to a reference sequence “GTATA”.

The following terms are used to describe the sequence relationships between two or more polynucleotides: “reference sequence”, “comparison window”, “sequence identity”, “percentage of sequence identity”, and “substantial identity”. A “reference sequence” is a defined sequence used as a basis for a sequence comparison; a reference sequence may be a subset of a larger sequence, for example, as a segment of a full-length cDNA or gene sequence given in a sequence listing, or may comprise a complete cDNA or gene sequence. Generally, a reference sequence is at least 20 nucleotides in length, frequently at least 25 nucleotides in length, and often at least 50 nucleotides in length. Since two polynucleotides may each (1) comprise a sequence (i.e., a portion of the complete polynucleotide sequence) that is similar between the two polynucleotides, and (2) may further comprise a sequence that is divergent between the two polynucleotides, sequence comparisons between two (or more) polynucleotides are typically performed by comparing sequences of the two polynucleotides over a “comparison window” to identify and compare local regions of sequence similarity.

A “comparison window”, as used herein, refers to a conceptual segment of at least 20 contiguous nucleotides and wherein the portion of the polynucleotide sequence in the comparison window may comprise additions or deletions (i.e., gaps) of 20 percent or less as compared to the reference sequence (which does not comprise additions or deletions) for optimal alignment of the two sequences. Optimal alignment of sequences for aligning a comparison window may be conducted by using local homology algorithms or by a search for similarity method, by computerized implementations of these algorithms (GAP, BESTFIT, FASTA, and TFASTA Genetics Software Package or by inspection, and the best alignment (i.e., resulting in the highest percentage of homology over the comparison window) generated by the various methods is selected.

The term “sequence identity” means that two polynucleotide sequences are identical (i.e., on a nucleotide-by-nucleotide basis) over the window of comparison. The term “percentage of sequence identity” means that two polynucleotide sequences are identical (i.e., on a nucleotide-by-nucleotide basis) over the window of comparison. The term “percentage of sequence identity” is calculated by comparing two optimally aligned sequences over the window of comparison, determining the number of positions at which the identical nucleic acid base (e.g., A, T, C, G, U, or I) occurs in both sequences to yield the number of matched positions, dividing the number of matched positions by the total number of positions in the window of comparison (i.e., the window size), and multiplying the result by 100 to yield the percentage of sequence identity. The terms “substantial identity” as used herein denote a characteristic of a polynucleotide sequence, wherein the polynucleotide comprises a sequence that has at least 85 percent sequence identity, preferably at least 90 to 95 percent sequence identity, more usually at least 99 percent sequence identity as compared to a reference sequence over a comparison window of at least 20 nucleotide positions, frequently over a window of at least 20-50 nucleotides, wherein the percentage of sequence identity is calculated by comparing the reference sequence to the polynucleotide sequence which may include deletions or additions which total 20 percent or less of the reference sequence over the window of comparison.

As applied to polypeptides, the term “substantial identity” means that two peptide sequences, when optimally aligned, such as by the programs GAP or BESTFIT using default gap weights, share at least about 80% sequence identity, preferably at least about 90% sequence identity, more preferably at least about 95%percent sequence identity, and most preferably at least about 99% sequence identity.

A “protective immune response” and “prophylactic immune response” are used interchangeably to refer to an immune response which targets an immunogen to which the individual has not yet been exposed or targets a protein associated with a disease in an individual who does not have the disease, such as a tumor associated protein in a patient who does not have a tumor.

A “therapeutic immune response” refers to an immune response which targets an immunogen to which the individual has been exposed or a protein associated with a disease in an individual who has the disease.

The term “prophylactically effective amount” is meant to refer to the amount necessary to, in the case of infectious agents, prevent an individual from developing an infection, and in the case of diseases, prevent an individual from developing a disease.

The term “therapeutically effective amount” is meant to refer to the amount necessary to, in the case of infectious agents, reduce the level of infection in an infected individual in order to reduce symptoms or eliminate the infection, and in the case of diseases, to reduce symptoms or cure the individual.

“Inducing an immune response against an immunogen” is meant to refer to induction of an immune response in a naive individual and induction of an immune response in an individual previously exposed to an immunogen wherein the immune response against the immunogen is enhanced.

As used herein, “substantially pure” means an object species is the predominant species present (i.e., on a molar basis it is more abundant than any other individual species in the composition), and preferably a substantially purified fraction is a composition wherein the object species comprises at least about 50 percent (on a molar basis) of all macromolecular species present. Generally, a substantially pure composition will comprise more than about 80 percent of all macromolecular species present in the composition, more preferably more than about 85%, about 90%, about 95%, and about 99%. Most preferably, the object species is purified to essential homogeneity (contaminant species cannot be detected in the composition by conventional detection methods) wherein the composition consists essentially of a single macromolecular species.

“Transfected,” “transformed” or “transgenic” is used herein to include any host cell or cell line, which has been altered or augmented by the presence of at least one recombinant DNA sequence. The host cells of the present invention are typically produced by transfection with a DNA sequence in a plasmid expression vector, as an isolated linear DNA sequence, or infection with a recombinant viral vector.

### Coronavirus, S Protein and Vaccines Therefor

Coronaviruses are a frequent cause of respiratory infections in humans, and most people have been infected by the four known human coronaviruses (hCoV), namely, hCoV-HKU1, -OC43, -229E, and -NL63, by age 10. The immune responses to hCoV infections wane over time, and infection with a hCoV confers no or limited protection against other hCoVs. Consequently, multiple infections with hCoVs occur during a lifetime. Infections with hCoVs are typically mild, which is why vaccines to these viruses have not been developed.

The notion that coronaviruses do not pose a substantial risk for public health changed with the SARS-CoV outbreak in 2002-2003, which resulted in more than 8,000 human infections and 774 deaths. Most likely, the virus was transmitted to humans from bats (via palm civets as an intermediate host), demonstrating that the large reservoir of non-human coronaviruses poses a threat to human health. The outbreak was brought under control through quarantine measures. With the control of the SARS-CoV outbreak, vaccine development came to a halt at an early stage. Another example of the zoonotic potential of coronaviruses is MERS-CoV (Middle-East respiratory syndrome coronavirus) infections, which have occurred in the Middle East since 2012, with more than 2,400 confirmed cases and a case fatality rate of 30%.

The SARS-CoV-2 virus that emerged in late 2019 possesses all of the characteristics of a pandemic virus: it infects human cells and replicates in them efficiently, it is transmitted efficiently among humans, and it is antigenically novel to the human immune system. Asymptomatic infections occur in some people. Among the symptomatic infections, about 80% are mild with influenza like-symptoms. However, the remaining 20% cause severe disease often leading to acute respiratory distress syndrome and death. The case fatality rate (estimated to be 2%-3%) is much higher in the elderly and in patients with comorbidities.

The family Coronaviridae is composed of viruses with a positive-sense, non-segmented, single-stranded RNA genome, which is infectious upon entering a host cell. The subfamily Orthocoronaviridae can be further divided into four genera (alpha-, beta-, gamma-, and deltacoronaviruses). Within the genus betacoronavirus, five subgenera are currently recognized. SARS-CoV and SARS-CoV-2 belong to the subgenus Sarbecovirus (formerly called lineage B), whereas MERS-CoV belongs to the subgenus Merbecovirus (formerly called lineage C). The four hCoVs belong to the genus alphacoronavirus (hCoV-NL63 and -229E) and betacoronavirus, subgenus Embecovirus (formerly called lineage A; hCoV-OC43 and -HKU1).

To date, human infections have been caused by coronaviruses of two genera, alpha- and betacoronaviruses, with all zoonotic events being caused by betacoronaviruses, specifically those of the subgenus Sarbecovirus. The natural reservoirs of betacoronaviruses are believed to be bats (Sarbecoviruses) and rodents (Embecoviruses), although betacoronaviruses have been isolated from other mammalian species. Based on genetic and phylogenetic analyses, SARS-CoV and SARS-CoV-2 originated from bats and were transmitted to humans directly or through an intermediate host. MERS-CoV appears to be zoonotic in dromedary camels in the Middle East and occasionally transmits to humans.

The rapid development (Baden et al., 2021; Polack et al., 2020; Sadoff et al., 2021; Voysey et al., 2021) and deployment of vaccines is helping to bring the pandemic under control in parts of the world. Most currently approved vaccines target the spike (S) protein on the surface of SARS-CoV-2 and generate a neutralizing antibody response that primarily targets the receptor-binding domain (RBD). While these S-based vaccines are currently effective, mutations to the S protein have already been shown to reduce vaccine efficacy (Garcia-Beltran et al., 2021; Madhi et al., 2021; Wang et al., 2021). Furthermore, there is potential for the transmission of other zoonotic coronaviruses to humans, which could result in epidemic disease (Menachery et al., 2015; Menachery et al., 2016).

The spike protein of coronavirus is composed of the head and stem regions. The head harbors immunodominant epitopes whose sequences are highly variable among coronaviruses. Vaccine candidates that elicit more broadly reactive immune response than control vaccines are tested for their protective efficacy in challenge studies in wild-type or transgenic mice expressing a human coronavirus receptor, and in Syrian hamsters, a robust small animal model for SARS-CoV-2. The immunization and challenge studies are complemented by studies to test the durability of the immune responses in vaccinated animals, and to test the prevention of virus transmission from and to vaccinated animals. Highly effective vaccines typically stimulate both B and T cell responses. B cell responses are essential to elicit protective antibodies that neutralize the virus or work through other mechanisms. Cytotoxic responses stimulated by CD8 T cells are important to control virus infection and alleviate disease symptoms. Both the B cell and cytotoxic T cell responses require the help of CD4 T cells.

Protection against virus infection is conferred by neutralizing antibodies. However, the clearance of respiratory viral infections also relies on CD8 T cells, and on helper CD4 T cells for the generation of high affinity, mature B cell responses. An appreciable number of CD4 and CD8 epitopes have now been identified in the SARS-CoV-2 spike protein, many of which have considerable immunodominance in the overall antiviral response. The specificity and quality of cross-reactive T cell responses elicited by vaccine candidates can be assessed and compared with those in SARS-CoV-2-infected people.

Prior to 2019, vaccines to hCoVs have not been developed because of the relatively low impact of hCoV infections on public health. Several SARS- and MERS-CoV vaccine candidates (including whole-inactivated, subunit, DNA, mRNA, viral-vectored, and live attenuated vaccines) have been tested in animal models, but no vaccines are available for human use. The SARS-CoV-2 pandemic spurred an unprecedented effort to develop a vaccine, with more than 50 candidates in human clinical trials, and more than 160 in pre-clinical development (https://www.who.int/publications/m/item/draft-landscape-of-covid-19-candidate-vaccines). Candidates in clinical trials include inactivated and live-attenuated virus vaccines; replicating and non-replicating viral-vectored vaccines; protein subunit, RNA, and DNA vaccines; and virus-like particle (VLP) vaccines.

### EXEMPLARY EMBODIMENTS

In one embodiment, the disclosure provides for a VLP vaccine platform based on a self-assembling bacteriophage protein. The coat protein of MS2 forms dimers that self-assemble into an icosahedral capsid. This capsid can be used to deliver DNA or RNA or an antigen. Moreover, the loop region between the A and B dimers of the coat protein can be modified to conjugate with an antigen of interest. The resulting VLPs present multiple copies of the antigen,’ resulting in high immunogenicity. MS2-VLPs are easy to manufacture, relatively stable, and their size allows the effective presentation of the antigen to antigen-presenting cells. MS2-VLPs induce innate and humoral immune responses and are being tested as vaccines for foot-and-mouth disease virus and human papilloma virus. Preliminary studies with SARS-CoV-2 antigens showed that high antibody titers are induced in the serum of vaccinated animals, and that hamsters are protected against SARS-CoV-2 challenge after a single immunization with MS2-VLPs expressing the SARS-CoV-2 S protein. Based on these findings, MS2-VLPs will be used in RP1 to test novel SARS-CoV-2 antigen designs.

The S protein is the major coronaviral antigen, which facilitates virus binding to the cellular receptor and mediates the fusion between the viral and cellular membranes to release the viral genome into infected cells. The S protein comprises two noncovalently bound subunits (S1 and S2) that mediate binding to the host cell receptor and fusion of the viral and cellular membranes, respectively. The S1 subunit contains an N-terminal domain (NTD) and the receptor-binding domain (RBD), which mediates binding to the cellular receptor. The amino acids that interact with human angiotensin-converting enzyme 2 (hACE2), the cellular receptor of SARS-CoV-2, are located in the receptor-binding motif (RBM). The S2 subunit comprises the fusion peptide and two so-called heptad repeats that are important for the fusion process. Cryo-EM and X-ray crystallographic structures of SARS-CoV-2 S have been resolved, including structures bound to hACE2 or in complex with mAbs. The RBDs of SARS-CoV and -CoV-2 share 73% homology, with a higher degree of homology in the NTD of the RBD (~84%) and a lower degree of homology in the RBM (~48%). The NTDs of SARS-CoV and -CoV2 share ~53% homology.

Most neutralizing mAbs bind to the RBM and block SARS-CoV-2 binding to hACE2. Typically, these mAbs lack cross-reactivity with other coronaviruses. Additional epitopes are located in the RBD outside the RBM, and in the NTD; mAbs targeting these epitopes may neutralize virus infection by competing with hACE2 binding or through other mechanisms. Two epitopes in the head region (outside the RBM) elicit mAbs that react with closely related coronaviruses, suggesting that epitope-redesign may broaden immune responses. Several mAbs bind to the S2 region of the S protein (coronavirus antibody database; http://opig.stats.ox.ac.uk/webapps/covabdad), which is more conserved in sequence than the RBD and the NTD, resulting in more cross-reactive mAbs. The candidate antigens are based on the SARS-CoV-2 spike protein; those encoding full-length S may possess six stabilizing proline residues and a modified sequence at the S1/S2 cleavage site to replace the multiple basic amino acid residues with a single basic amino acid.

In one platform, the candidate antigens are presented to the immune system by VLPs based on the self-assembling coat protein of bacteriophage MS2 that may be modified with an AviTag (e.g., GLNIDFEAQKIEWHE) inserted in a surface loop. The inserted AviTag allows for site-specific biotinylation of the coat protein. The self-assembled, biotinylated MS2-VLPs were purified by size exclusion chromatography (SEC) and characterized by analytical SEC and dynamic light scattering (DLS) as described hereinbelow. The self-assembled VLPs were uniform in size and approximately 50 nm in diameter. Wild-type or mutant S proteins, or portions thereof, are expressed in cells such as E. coli cells, purified by using immobilized metal affinity chromatography, biotinylated, repurified, and mixed with the MS2-VLPs to form MS2-VLPs displaying spike proteins. The resulting preparations are characterized by using SEC, DLS, and ELISA. MS2-VLPs expressing wild-type SARS-CoV-2 S protein, after a single immunization protected Syrian hamsters against challenge with SARS-CoV-2.

To test the immunogenicity of vaccines, wild-type or transgenic mice expressing a receptor used by the coronavirus are immunized 2-3 times with the MS2-VLP vaccines. After each immunization, serum samples are tested in an ELISA for the depth and breadth of the immune responses. Importantly, serum and tissue samples are provided for a detailed analysis of the B- and T-cell responses.

The choice of the animal model is based on the challenge virus. For coronaviruses that utilize the human ACE2 receptor, transgenic hACE2 mice from Jackson Laboratories are used: however, coronavirus infection of these mice can result in viral encephalitis, which is typically not observed in infected people. For coronaviruses that bind to DPP4, transgenic mice expressing human DPP4 are obtained. Alternatively, wild-type mice transduced with adeno-associated virus serotype 9 expressing the appropriate human cellular receptor, or mouse-adapted coronaviruses may be used or generated as needed. The challenge viruses can be generated as authentic viruses, or as recombinant viruses with the spike protein of the coronavirus of interest in the genetic background of SARS-CoV-2.

The anti-spike CD4 and CD8 responses against candidate antigens may be assessed.

One described vaccine platform leverages a phage protein that self-assembles into VLPs and displays antigens at high valency to the immune system.

### Influenza Vaccines

A VLP vaccine of the invention includes an influenza HA displayed in its native orientation on a particle, e.g., a nanoparticle, formed of heterologous components. That particle may include one or more different HAs, e.g., two of more subtypes such as H1, H2, H3, H4, H5, H8, H7, H8, H9, H10, H11, H12, H13, H14, H15, H16, H17, or H18, or any combination thereof, two, three, four or five of H1, H2, H3, H4, H5, H6, H7, H8, H9, H10, H11, H12, H13, H14, H15, H16, H17, or H18 may be combined, e.g., particles displaying H1 and particles displaying H5, or particles displaying two different H1 isolated may be employed. In addition a particle may be combined with one or more isolated viruses including other isolated influenza viruses, one or more immunogenic proteins or glycoproteins of one or more isolated influenza viruses or one or more other pathogens, e.g., an immunogenic protein from one or more bacteria, non-influenza viruses, yeast or fungi, or isolated nucleic acid encoding one or more viral proteins (e.g., DNA vaccines) including one or more immunogenic proteins of the isolate having the HA displayed on the particle. In one embodiment, the influenza viruses of the invention may be vaccine vectors for influenza virus or other pathogens.

The vaccine, e.g., if multivalent, may include a component that is inactivated, e.g., using formalin or beta-propiolactone, for instance.

Forms of components other than the particles that may be included with the vaccine are described below

A subunit vaccine comprises purified glycoproteins. Such a vaccine may be prepared as follows: using viral suspensions fragmented by treatment with detergent, the surface antigens are purified, by ultracentrifugation for example. The subunit vaccines thus contain mainly HA protein, and also NA. The detergent used may be cationic detergent for example, such as hexadecyl trimethyl ammonium bromide (Bachmeyer, 1975), an anionic detergent such as ammonium deoxycholate (Laver & Webster, 1976); or a nonionic detergent such as that commercialized under the name TRITON X100. The hemagglutinin may also be isolated after treatment of the virions with a protease such as bromelin, and then purified. The subunit vaccine may be combined with an attenuated virus in a multivalent vaccine.

A split vaccine comprises virions which have been subjected to treatment with agents that dissolve lipids. A split vaccine can be prepared as follows: an aqueous suspension of the purified virus obtained as above, inactivated or not, is treated, under stirring, by lipid solvents such as ethyl ether or chloroform, associated with detergents. The dissolution of the viral envelope lipids results in fragmentation of the viral particles. The aqueous phase is recuperated containing the split vaccine, constituted mainly of hemagglutinin and neuraminidase with their original lipid environment removed, and the core or its degradation products. Then the residual infectious particles are inactivated if this has not already been done. The split vaccine may be combined with an attenuated virus in a multivalent vaccine.

Inactivated Vaccines. Inactivated influenza virus vaccines are provided by inactivating replicated virus using known methods, such as, but not limited to, formalin or β-propiolactone treatment. Inactivated vaccine types that can be used in the invention can include whole-virus (WV) vaccines or subvirion (SV) (split) vaccines. The WV vaccine contains intact, inactivated virus, while the SV vaccine contains purified virus disrupted with detergents that solubilize the lipid-containing viral envelope, followed by chemical inactivation of residual virus.

In addition, vaccines that can be used include those containing the isolated HA and NA surface proteins, which are referred to as surface antigen or subunit vaccines.

Live Attenuated Virus Vaccines. Live, attenuated influenza virus vaccines can be used for preventing or treating influenza virus infection. Attenuation may be achieved in a single step by transfer of attenuated genes from an attenuated donor virus to a replicated isolate or reassorted virus according to known methods. Since resistance to influenza A virus is mediated primarily by the development of an immune response to the HA and/or NA glycoproteins, the genes coding for these surface antigens come from the reassorted viruses or clinical isolates. The attenuated genes are derived from an attenuated parent. In this approach, genes that confer attenuation generally do not code for the HA and NA glycoproteins.

Viruses (donor influenza viruses) are available that are capable of reproducibly attenuating influenza viruses, e.g., a cold adapted (ca) donor virus can be used for attenuated vaccine production. Live, attenuated reassortant virus vaccines can be generated by mating the ca donor virus with a virulent replicated virus. Reassortant progeny are then selected at 25° C. (restrictive for replication of virulent virus), in the presence of an appropriate antiserum, which inhibits replication of the viruses bearing the surface antigens of the attenuated ca donor virus. Useful reassortants are: (a) infectious, (b) attenuated for seronegative non-adult mammals and immunologically primed adult mammals, (c) immunogenic and (d) genetically stable. The immunogenicity of the ca reassortants parallels their level of replication. Thus, the acquisition of the six transferable genes of the ca donor virus by new wild-type viruses has reproducibly attenuated these viruses for use in vaccinating susceptible mammals both adults and non-adult.

Other attenuating mutations can be introduced into influenza virus genes by site-directed mutagenesis to rescue infectious viruses bearing these mutant genes. Attenuating mutations can be introduced into non-coding regions of the genome, as well as into coding regions. Such attenuating mutations can also be introduced into genes other than the HA or NA, e.g., the PB2 polymerase gene. Thus, new donor viruses can also be generated bearing attenuating mutations introduced by site-directed mutagenesis, and such new donor viruses can be used in the production of live attenuated reassortants vaccine candidates in a manner analogous to that described above for the ca donor virus. Similarly, other known and suitable attenuated donor strains can be reassorted with influenza virus to obtain attenuated vaccines suitable for use in the vaccination of mammals.

In one embodiment, such attenuated viruses maintain the genes from the virus that encode antigenic determinants substantially similar to those of the original clinical isolates. This is because the purpose of the attenuated vaccine is to provide substantially the same antigenicity as the original clinical isolate of the virus, while at the same time lacking pathogenicity to the degree that the vaccine causes minimal chance of inducing a serious disease condition in the vaccinated mammal.

The viruses in a multivalent vaccine can thus be attenuated or inactivated, formulated and administered, according to known methods, as a vaccine to induce an immune response in an animal, e.g., a mammal. Methods are well-known in the art for determining whether such attenuated or inactivated vaccines have maintained similar antigenicity to that of the clinical isolate or high growth strain derived therefrom. Such known methods include the use of antisera or antibodies to eliminate viruses expressing antigenic determinants of the donor virus; chemical selection (e.g., amantadine or rimantidine); HA and NA activity and inhibition; and nucleic acid screening (such as probe hybridization or PCR) to confirm that donor genes encoding the antigenic determinants (e.g., HA or NA genes) are not present in the attenuated viruses.

### Other Vaccines

Other vaccines having an immunogenic protein of other viruses, bacteria or fungi may be displayed using the platforms disclosed herein. For example, Varicella Zoster Virus glycoprotein E, Ebolavirus glycoprotein, Dengue virus envelope and/or premembrane proteins, HIV envelope proteins (gp), Bordetella pertussis pertactin, or Plasmodium circumsporozoite protein may be displayed using the systems disclosed herein.

### Pharmaceutical Compositions

Pharmaceutical composition, suitable for inoculation, e.g., nasal, parenteral or oral administration, such as by intravenous, intramuscular, intranasal, topical or subcutaneous routes, comprise one or more nanoparticles, optionally further comprising sterile aqueous or non-aqueous solutions, suspensions, and emulsions. The compositions can further comprise auxiliary agents or excipients, as known in the art. The composition is generally presented in the form of individual doses (unit doses). Preparations for parenteral administration include sterile aqueous or non-aqueous solutions, suspensions, and/or emulsions, which may contain auxiliary agents or excipients known in the art. Examples of non-aqueous solvents are propylene glycol, polyethylene glycol, vegetable oils such as olive oil, and injectable organic esters such as ethyl oleate. Carriers or occlusive dressings can be used to increase skin permeability and enhance antigen absorption. Liquid dosage forms for oral administration may generally comprise a liposome solution containing the liquid dosage form. Suitable forms for suspending liposomes include emulsions, suspensions, solutions, syrups, and elixirs containing inert diluents commonly used in the art, such as purified water. Besides the inert diluents, such compositions can also include adjuvants, wetting agents, emulsifying and suspending agents, or sweetening, flavoring, or perfuming agents.

When a composition is used for administration to an individual, it can further comprise salts, buffers, adjuvants, or other substances which are desirable for improving the efficacy of the composition. For vaccines, adjuvants, substances which can augment a specific immune response, can be used. Normally, the adjuvant and the composition are mixed prior to presentation to the immune system, or presented separately, but into the same site of the organism being immunized.

In one embodiment, the pharmaceutical composition is part of a controlled release system, e.g., one having a pump, or formed of polymeric materials (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger & Peppas, J. Macromol. Sci. Rev. Macromol. Chem., 23:61 (1983); see also Levy et al., Science, 228:190 (1985); During et al., Ann. Neurol., 25:351 (1989); Howard et al., J. Neurosurg., 71:105 (1989)). Other controlled release systems are discussed in the review by Langer Science, 249:1527 (1990)).

The pharmaceutical compositions comprise a therapeutically effective amount of the nanoparticles, and a pharmaceutically acceptable carrier. In a specific embodiment, the term “pharmaceutically acceptable” means approved by a regulatory agency of the Federal or a state government or listed in the U.S. Pharmacopeia or other generally recognized pharmacopeiae for use in animals, and more particularly in humans. The term “carrier” refers to a diluent, adjuvant, excipient, or vehicle with which the pharmaceutical composition is administered. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. These compositions can take the form of solutions, suspensions, emulsion, tablets, pills, capsules, powders, sustained-release formulations and the like. These compositions can be formulated as a suppository. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington’s Pharmaceutical Sciences” by E. W. Martin. Such compositions will contain a therapeutically effective amount of the nanoparticles, preferably in purified form, together with a suitable amount of carrier so as to provide the form for proper administration to the patient. The formulation should suit the mode of administration.

The compositions may be systemically administered, e.g., orally or intramuscularly, in combination with a pharmaceutically acceptable vehicle such as an inert diluent. For oral administration, the nanoparticles may be combined with one or more excipients and used in the form of ingestible capsules, elixirs, suspensions, syrups, wafers, and the like. Such compositions should contain at least 0.1% of active compound. The percentage of the compositions and preparations may, of course, be varied and may conveniently be between about 2 to about 60% of the weight of a given unit dosage form. The amount of active compound in such useful compositions is such that an effective dosage level will be obtained.

The compositions may also contain the following: binders such as gum tragacanth, acacia, com starch or gelatin; excipients such as dicalcium phosphate; a disintegrating agent such as com starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. Various other materials may be present. For instance, a syrup or elixir may contain the nanoparticles, sucrose or fructose as a sweetening agent, methyl and propylparabens as preservatives, a dye and flavoring such as cherry or orange flavor. Of course, any material used in preparing any unit dosage form, including sustained-release preparations or devices, should be pharmaceutically acceptable and substantially non-toxic in the amounts employed.

The composition also be administered intravenously or intraperitoneally by infusion or injection. Solutions of the nanoparticles can be prepared in water or a suitable buffer, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of undesirable microorganisms.

The pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of undesirable microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride.

Sterile injectable solutions are prepared by incorporating the nanoparticles in the required amount in the appropriate solvent with various of the other ingredients enumerated above, as required, followed by filter sterilization.

Useful liquid carriers include water, alcohols or glycols or water-alcohol/glycol blends, in which the present nanoparticles can be dissolved or dispersed at effective levels, optionally with the aid of non-toxic surfactants. Adjuvants such as fragrances and additional antimicrobial agents can be added to enhance the properties for a given use. The resultant liquid compositions can be applied from absorbent pads, used to impregnate bandages and other dressings, or sprayed onto the affected area using pump-type or aerosol sprayers.

Useful dosages can be determined by comparing their in vitro activity and in vivo activity in animal models.

### Pharmaceutical Purposes

The administration of the composition may be for either a “prophylactic” or “therapeutic” purpose. When provided prophylactically, the compositions of the invention which are vaccines are provided before any symptom or clinical sign of a pathogen infection becomes manifest. The prophylactic administration of the composition serves to prevent or attenuate any subsequent infection. When provided prophylactically, the gene therapy compositions of the invention, are provided before any symptom or clinical sign of a disease becomes manifest. The prophylactic administration of the composition serves to prevent or attenuate one or more symptoms or clinical signs associated with the disease.

When provided therapeutically, a vaccine is provided upon the detection of a symptom or clinical sign of actual infection. The therapeutic administration of the compound(s) serves to attenuate any actual infection. When provided therapeutically, a composition is provided upon the detection of a symptom or clinical sign of the disease. The therapeutic administration of the compound(s) serves to attenuate a symptom or clinical sign of that disease.

Thus, a vaccine composition of the present invention may be provided either before the onset of infection (so as to prevent or attenuate an anticipated infection) or after the initiation of an actual infection. Similarly, the composition may be provided before any symptom or clinical sign of a disorder or disease is manifested or after one or more symptoms are detected.

A composition is said to be “pharmacologically acceptable” if its administration can be tolerated by a recipient mammal. Such an agent is said to be administered in a “therapeutically effective amount” if the amount administered is physiologically significant. A composition of the present invention is physiologically significant if its presence results in a detectable change in the physiology of a recipient patient, e.g., enhances at least one primary or secondary humoral or cellular immune response against at least one strain of a virus.

The “protection” provided need not be absolute, i.e., the viral infection need not be totally prevented or eradicated, if there is a statistically significant improvement compared with a control population or set of mammals. Protection may be limited to mitigating the severity or rapidity of onset of symptoms or clinical signs of the virus infection.

### Pharmaceutical Administration

A composition of the present invention may confer resistance by either passive immunization or active immunization. In active immunization, a live vaccine composition is administered prophylactically to a host (e.g., a mammal), and the host’s immune response to the administration protects against infection and/or disease. For passive immunization, the elicited antisera can be recovered and administered to a recipient suspected of having an infection caused by at least one virus strain.

The present invention thus includes methods for preventing or attenuating a disorder or disease, e.g., an infection by at least one strain of coronavirus. As used herein, a vaccine is said to prevent or attenuate a disease if its administration results either in the total or partial attenuation (i.e., suppression) of a clinical sign or condition of the disease, or in the total or partial immunity of the individual to the disease.

At least one composition of the present invention, may be administered by any means that achieve the intended purposes. For example, administration of such a composition may be by various parenteral routes such as subcutaneous, intravenous, intradermal, intramuscular, intraperitoneal, intranasal, oral or transdermal routes. Parenteral administration can be accomplished by bolus injection or by gradual perfusion over time.

A typical regimen for preventing, suppressing, or treating a viral related pathology, comprises administration of an effective amount of a vaccine composition as described herein, administered as a single treatment, or repeated as enhancing or booster dosages, for instance, over a period up to and including between one week and about 24 months, or any range or value therein.

According to the present invention, an “effective amount” of a composition is one that is sufficient to achieve a desired effect. It is understood that the effective dosage may be dependent upon the species, age, sex, health, and weight of the recipient, kind of concurrent treatment, if any, frequency of treatment, and the nature of the effect wanted. The ranges of effective doses provided below are not intended to limit the invention and represent dose ranges.

Exemplary doses include but are not limited to from about 10⁴ to 10⁸ ng, 10⁸ to 10⁸ ng, 10⁶ to 10¹⁰ ng, or 10⁸ to 10¹² ng, or more, or from about 10⁸ to 10⁸ particles, 10⁸ to 10¹⁰ particles, or 10¹⁰ to 10¹² particles or more. In one embodiment,a dose is from about 10¹ to 10⁸ µg, 10² to 10⁶ µg, 10³ to 10⁵ µg, or 10⁴ to 10¹⁶ µg.

For an influenza vaccine, the dose may range from about 0.1 to 1000, e.g., 30 to 100 µg, of HA protein. However, the dosage should be a safe and effective amount as determined by conventional methods, using existing vaccines as a starting point. For example, the dosage of immunoreactive HA in each dose may be standardized to contain a suitable amount, e.g., 30 to 100 µg or any range or value therein, or the amount recommended by government agencies or recognized professional organizations. In one embodiment, a suitable amount may be, e.g., 1-50 µg or any range or value therein, or the amount recommended by the U.S. Public Heath Service (PHS), which is usually 15 µg per component for older children (greater than or equal to 3 years of age), and 7.5 µg per component for children less than 3 years of age. In one embodiment the dose may be from about 10 µg to about 50 µg, e.g., 15 µg to 45 µg, of HA.

### EXEMPLARY ADJUVANTS

Adjuvants include but are not limited to aluminum, water in oil (W/O) emulsions, oil in water (O/W) emulsions, ISCOM, liposomes, nano- or micro-particles, muramyl di- and/or tripeptides, saponin, non-ionic block co-polymers, lipid A, cytokines, bacterial toxins, carbohydrates, and derivatized polysaccharides and a combination of two or more these adjuvants in an Adjuvant System (AS).

Exemplary classes of adjuvants include but are not limited to agonists of TLR3, e.g., poly (I:C), agonists of TLR4, e.g., one or more components of bacterial lipopolysaccharide, e.g., monophosphoryl lipid A (MPLA), MPL®, and synthetic derivatives, e.g., E6020,agonists of TLR5, e.g., bacterial flagellin), agonists of TLR7, 8, e.g., single stranded RNA or imidazoquinolines (e.g., imiquimod, gardiquimod and R848),agonists of TLR9, e.g., CpG oligonucleotides and ISS immunostimulatory sequences, as well as imidazoquinolines, agonists of the NLRP3 inflammasome, e.g., chitosan, and dual TLR½ agonists, e.g., Pam3CSK4, a lipopeptide.

In one embodiment, the adjuvant comprises saponin, a natural product derived from tree bark, which may be combined with cholesterol or a cholesterol like molecule, e.g., squalene.

In one embodiment, the adjuvant comprises an oil-in-water (O/W) emulsion comprising, for example, MF59 or AS03 and optionally 2% squalene. In one embodiment, the adjuvant comprises two different adjuvants, e.g., MPL and a saponin such as QS21, for example, in liposome.

In one embodiment, the adjuvant comprises Freund’s Incomplete Adjuvant (IFA), MF59®, GLA-SE, IC31®, CAF01 AS03, AS04, or ISA51, and may include α-tocopherol, squalene and/or polysorbate 80 in an oil-in-water emulsion.

In one embodiment, the adjuvant comprises extracts and formulations prepared from Ayurvedic medicinal plants including but not limited to Withania somnifera, Emblica officinalis, Panax notoginseng, Tinospora cordifolia or Asparagus racemosus.

In one embodiment, the adjuvant comprises aluminum salts, saponin, muramyl di- and/or tripeptides, Bordetella pertussis, and/or cytokines.

In one embodiment, the adjuvant is not alum or an aluminum salt.

In one embodiment, the adjuvant is mixed with the nanoparticles just prior to administration.

The invention will be described by the following non-limiting examples.

### Example 1

Coronaviruses are enveloped, single-stranded RNA viruses that belong to the family Coronaviridae. The subfamily Orthocoronaviridae comprises four genera, alpha-, beta-, gamma-, and deltacoronavirus. To date, all human infections have been caused by alpha- and betacoronaviruses. The currently recognized four human coronaviruses (hCoV) (two alphacoronaviruses, hCoV-NL63 and -229E, and two betacoronavirus, hCoV-OC43, -HKU1) cause an appreciable proportion of all respiratory infections in humans with typically mild to moderate symptoms (‘common cold’). Because of the mild nature of the disease caused by these viruses, coronaviruses were not considered a severe public health threat until 2002, when a novel betacoronavirus originating from animals (SARS-CoV) caused more than 8,000 infections and 774 deaths. Closely related viruses have been identified in bats, including Bat-CoV WIV1 and SHC014 (which will be used in this project to test novel SARS-CoV-2-based antigens). MERS-CoV (also a betacoronavirus) was first described in Saudi-Arabia in 2012 and has infected more than 2,400 people with a case fatality rate of about 30%. Despite the appreciable number of human infections, SARS-CoV and MERS-CoV have not caused pandemic outbreaks.

The S protein, the major coronaviral antigen, comprises two non-covalently bound subunits (S1 and S2) that interact with the cellular receptor (S1) and mediate the fusion of the viral and cellular membranes (S2). Binding to the human angiotensin-converting enzyme 2 (hACE2), the cellular receptor of SARS-CoV-2, is mediated by the receptor-binding motif (RBM, amino acids 438-498 (9)), within the receptor-binding domain (RBD, amino acids 319-541. The RBD is located in S1, together with the N-terminal domain (NTD, amino acids 14-305. The RBDs of SARS-CoV and SARSCoV-2 share 73% homology, but only 43% of the amino acids in the RBM are conserved between the two viruses; the homology between SARS-CoV-2 and MERS-CoV is considerably lower. The NTDs of SARS-CoV and -CoV2 share 53% homology.

Structural analysis and epitope mapping have identified several antigenic regions in the S protein: (i) RBM: Many neutralizing monoclonal antibodies (mAbs) bind to the RBM and block the interaction with hACE2, resulting in virus neutralization. Because of the substantial sequence diversity in the RBM among coronaviruses, these epitopes are typically virus-specific. (ii) RBD outside the RBM: Several mAbs bind to the RBD outside the RBM and may neutralize virus infection through different mechanisms. Two relatively conserved epitopes are recognized by mAbs (e.g., CR3022 and A309) that cross-react with closely related coronaviruses. (iii) NTD: To date, only one epitope has been characterized in detail in the NTD of the SARS-CoV-2 S protein (17). (iv) S2: Several mAbs bind to S2, and some of them are neutralizing (coronavirus antibody database; http://opig.stats.ox.ac.uk/webapps/covabdab) (22). S2 is more conserved than the RBD and the NTD, and several S2-specific mAbs react with heterologous coronaviruses, suggesting that conserved epitopes may be targeted to induce more broadly reactive antibodies.

The coat proteins of bacteriophages can self-assemble to form VLPs, which can be biochemically modified to present antigens in high valency to the host immune system. VLP vaccines are now licensed for several human papillomaviruses (HPV-9, HPV-16/18), hepatitis B and hepatitis E viruses, and against malaria. The bacteriophage MS2 consists of 180 monomeric coat proteins that self-assemble to form an icosahedral structure consisting of 90 homodimers. As described herein VLPs based on the MS2 coat protein as scaffolds were developed for the multivalent display of the SARS-CoV-2 S protein and shown to demonstrate protective efficacy in hamsters after a single immunization. MS2-VLPs are used to test antigens for their immunogenicity and protective efficacy in animal models. Exemplary antigens include but are not limited to the ectodomain of the spike protein, the S2 region of the spike protein, the RBD regions of the spike protein, or the NTD.

Vectors for MS2-VLPs are generated using a single-chain MS2 coat protein dimer wherein the second monomer may have an AviTag inserted in a surface loop. The tagged protein may be biotinylated, mixed with an excess of streptavidin, e.g., divalent, trivalent or tetravalent (or more) streptavidin,and purified. Biotinylated variants of the hybrid spike protein may be mixed with the streptavidin-tagged coat protein, resulting in MS2-VLPs displaying the SARS-CoV-2 S protein. The MS2-VLPs are purified by using established protocols.

Thus, in on embodiment, the MS2 coat protein that is modified to contain an AviTag is biotinylated, and the biotinylated MS2 is mixed with streptavidin (center panel), e.g., a tetravalent streptavidin, and incubated with biotinylated spike protein, resulting in MS2-VLPs displaying the spike protein or a portion thereof.

The protective efficacy of a vaccine is determined by the antibodies elicited upon vaccination. To test the antibody responses to, for example, the S proteins, mice are immunized by intramuscularly injecting them with 50 µg of the respective purified MS2-VLP in the presence of Alhydrogel (2% solution) in increase immunogenicity. Mice may be sequentially immunized with SARS-CoV-2 (to account for the fact that most people will have been infected with or vaccinated against SARS-CoV-2), followed by two immunizations of the nanoparticles.

### X-ray Crystallography and Cryo-EM

Synchrotron based X-ray crystallography and Cryo-EM are applied to analyze the specific antigens or antigen-antibody interactions. While both X-ray crystallography and high-resolution Cryo-EM offer three-dimensional insights, crystal structures allow us to understand specific interactions between epitopes and paratopes, which are often very subtle, particularly in the case of promiscuous interactions. The selected antigens are generated with a C-terminal hexa-histidine tag and purified by using immobilized metal affinity chromatography using Ni-ion media.

TABLE 1

| RBD IgG endpoint titer^{a} | | | | | | | Neutralizing antibody titer^{b} | | | | |
|---|---|---|---|---|---|---|---|---|---|---|---|
| Vaccine group | Animal # | Replicate 1 | Replicate 2 | Replicate 3 | Geometric mean | Geometric SD factor | Replicate 1 | Replicate 2 | Replicate 3 | Geometric mean | Geometric SD factor |
| PBS | 1 | <10 | <10 | <10 | | | <10 | <10 | <10 | | |
| | 2 | <10 | <10 | <10 | <10 | – | <10 | <10 | <10 | <10 | – |
| | 3 | <10 | <10 | <10 | | | <10 | <10 | <10 | | |
| MS2-SA VLP | 1 | <10 | <10 | <10 | | | <10 | <10 | <10 | | |
| | 2 | <10 | <10 | <10 | <10 | – | <10 | <10 | <10 | <10 | – |
| | 3 | <10 | <10 | <10 | | | <10 | <10 | <10 | | |
| VLP-S2_{Pro} | 1 | 20,480 | 40,960 | 20,480 | | | 320 | 640 | 320 | | |
| | 2 | 81,920 | 40,960 | 81,920 | 35,113 | 1.78 | 640 | 320 | 320 | 373 | 1.36 |
| | 3 | 20,480 | 20,480 | 40,960 | | | 320 | 320 | 320 | | |
| VLP-S_{6Pro} | 1 | 81,920 | 81,920 | 81,920 | | | 640 | 640 | 640 | | |
| | 2 | 81,920 | 81,920 | 81,920 | 70,225 | 1.36 | 640 | 640 | 640 | 549 | 1.36 |
| | 3 | 40,960 | 81,920 | 40,960 | | | 320 | 640 | 320 | | |

^{a}Viral antibody endpoint titers against the RBD (receptor-binding domain) from three independent assays (three animals in each group). Endpoint titers using 2-fold diluted sera were expressed as the reciprocal of the highest dilution with an optical density at 490 nm cutoff value >0.15; sera were collected on day 28 after immunization.
^{b}Viral neutralization titers from three independent assays (three animals in each group). Endpoint titers using twofold diluted sera were expressed as the reciprocal of the highest dilution that completely prevented cytopathic effects; sera were collected on day 28 after immunization.

### Preliminary Data

SARS-CoV-2 variant with a mutation in the spike protein (D614G) transmits faster among Syrian hamsters than the progenitor virus. Therefore, Syrian hamsters are a useful small animal model for SARS-CoV-2.

A single immunization with MS2-VLP-S resulted in appreciable levels of IgG antibodies against the RBD of the S protein and, more importantly, high neutralizing antibody titers ranging from 320-640. In contrast, as expected, anti-S antibodies were barely detectable in hamsters immunized with the controls (VLPs alone or PBS). Four weeks after immunization, the animals were intranasally inoculated with 10³ plaque-forming units of SARS-CoV-2. Three days after virus challenge, when virus levels in the lungs peak (100), the animals were sacrificed and lung and nasal turbinate samples were titrated (see below), As expected, animals in both control groups (PBS and MS2-VLP) had high viral loads in the lungs; however, in hamsters immunized with MS2-VLP-S, no infectious virus was detected in the lungs. Moreover, the MS2-VLP-S-immunized hamsters had lower virus titers in their nasal turbinates compared to the control animals. In the proposed project, we will use the multivalent display of engineered S variants from these VLP scaffolds to test novel coronavirus antigens.

Immunization studies are conducted by intramuscularly immunizing wild-type or genetically modified mice or Syrian hamsters with 10 µg of the MS2-VLPs. Per antigen, 12 animals are vaccinated (six of each sex). The schedule of immunizations may vary depending on the antigen candidate. Typically, a boost immunization is given 21 days after the previous immunization. If needed, a third immunization is given. After each immunization, sera is collected and tested in ELISAs.

Protective efficacy of MS2-VLP displaying SARS-CoV S in Syrian hamsters. Animals were immunized with PBS, control MS2-VLP, or MS2-VLPs displaying SARS-CoV-2 S containing 2 or 6 stabilizing proline residues. Twenty-eight days after immunization, the animals were infected with 10³ pfu of SARS-CoV-2. Three days later, virus titers were determined in the lungs (A) or nasal turbinates (B). Shown are the geometric means with geometric SD, n=3). † - No infectious virus was detected (detection limit, 10 PFU/g). ns: not statistically significant, *p < 0.1; ****p < 0.0001, determined by a oneway analysis of variance (ANOVA) and Dunnett’s post-hoc multiple comparison between groups (α = 0.1). Assumptions of the normality of residuals and homogeneity of variance were validated by using the Shapiro-Wilk test and the Brown-Forsythe test, respectively.

Immunized animals are infected by intranasal inoculation with 10³-10⁵ PFU of challenge virus 28 days after the final immunization. During the course of infection, body weights are recorded and the general health of the animals monitored. Three and six days after challenge, groups of animals (six animals at each timepoint for each vaccine, i.e., three of each sex) are euthanized to assess virus titers in the respiratory organs (nasal turbinate and lung samples). A portion of tissues are fixed for pathology. Six months after the last vaccination, animals are challenged with coronaviruses.

Syrian hamsters (non-immunized or immunized) are infected with challenge virus. Twenty-four hours later, naïve animals (either non-immunized or immunized) are placed into a neighboring cage. Both cages are housed in transmission units that allow for directional airflow and HEPA-filtered exhaust air. Four days after infection or exposure, animals are euthanized to assess virus titers in the respiratory organs (nasal turbinate and lung samples). A portion of tissues are fixed for pathology. These studies determine whether our vaccinate candidates can reduce or prevent virus transmission, even though they may not induce sterilizing immunity in vaccinated animals.

Thus, vaccine candidates are tested in animal models for their immunogenicity, durability of immune responses, and protective efficacy.

### Example 2

### Methods

### Expression and Purification of MS2

DNA encoding single-chain MS2 coat protein dimer with an AviTag inserted between the 14 and 15 residues of the second coat protein monomer was cloned into pET-28b between the Ndel and Xhol restrictions sites by GenScript Biotech Corporation (Piscataway, NJ). The MS2 dimer with the inserted AviTag was co-transformed with pAcm-BirA (Avidity LLC) into BL21(DE3) competent E. coli (New England Biolabs) according to the manufacturer’s instructions. The transformation was added to 5 mL of 2xYT media and grown overnight at 37° C. The 5-mL starter culture was then added to 1 L of 2xYT media, which was incubated shaking at 37° C. until induction with IPTG (1 M: GoldBio) at an OD of 0.6. Immediately after induction, biotin (50 µM) was added to the culture and the incubator temperature was reduced to 30° C. After overnight incubation, the culture was centrifuged for 7 min at 7000×g and the supernatant was decanted. The cell pellet was homogenized into 25 mL of 20 mM Tris Base (pH 8.0) supplemented with lysozyme (0.5 mg/mL; Alfa Aesar), a protease inhibitor tablet (Sigma-Aldrich), and benzonase (125 units; EMD Millipore). The resuspended cells were then kept on ice and stirred intermittently for 20 minutes. Sodium deoxycholate (Alfa Aesar) was then added to a final concentration of 0.1% (w/v), and the mixture was sonicated for 3 minutes at 35% amplitude with a pulse of 3 s on and 3 s off (Sonifier S-450, Branson Ultrasonics). The sonication was repeated after allowing the lysate to cool on ice for 2 minutes. Next, the lysed cells were centrifuged for 30 minutes at 27,000 × g. The supernatant was collected, and centrifuged again for 15 minutes at 12.000 × g. The resulting supernatant was then diluted 3-fold in 20 mM Tris Base and filtered with a 0.45-µm bottle-top filter (VWR). Then, 25 mL of the diluted lysate was loaded onto four HiScreen Capto Core 700 columns (Cytiva) in series using an AKTA start system. The columns were washed with ~3 column volumes of 20 mM Tris Base while fractions were collected. Fractions were subsequently analyzed for purity and recovery of MS2 by using SDS-PAGE. Desirable fractions were pooled, concentrated by using a 10 kDa MWCO centrifugal filter (Millipore Sigma), and further purified by using a Superdex 200 Increase 10/300 column (Cytiva). MS2 was quantified by using a bicinchoninic acid assay (BCA) (Thermo Scientific). Expression, refolding, and purification of streptavidin (SA)

SA was expressed, refolded, and purified essentially as previously described (Booth et al., 2011; Jung & Mun, 2018). Briefly, DNA encoding SA (Addgene plasmid #46367) (Fairhead et al., 2014) was transformed into BL21(DE3) cells (New England Biolabs) according to the manufacturer’s protocol. The transformation was split among four culture tubes each containing 5 mL of 2xYT media, which were incubated overnight at 37° C. Each 5 mL culture was added to one of four 1 L flasks of 2xYT and grown at 37° C. Upon reaching an OD of 0.6, expression of inclusion bodies was induced using IPTG (1 M; GoldBio) and the temperature of the incubator was reduced to 30° C. After incubation overnight, the culture was centrifuged for 7 minutes at 7000 × g such that 4 I of culture resulted in two cell pellets. Each pellet was resuspended in 50 mL of resuspension buffer (50 mM Tris, 100 mM NaCl, pH 8.0) supplemented with lysozyme (1 mg/mL; Alfa Aesar) and benzonase (500 units; EMD Millipore) and was allowed to incubate at 4° C. for 1 hour ith occasional mixing. These mixtures were then homogenized, brought to a concentration of 0.1% (w/v) sodium deoxycholate (Alfa Aesar), and sonicated (Sonifier S-450, Branson Ultrasonics) for 3 minutes at 35% amplitude with a pulse of 3 seconds on and 3 seconds off The resulting lysate was then centrifuged for 15 minutes at 27,000 × g. The supernatant was discarded, and the two pellets were each again resuspended in 50 mL of resuspension buffer supplemented with lysozyme. (1 m)g/mL; Alfa Aesar) and the lysis procedure was repeated. This procedure resulted in two inclusion body pellets, which were then washed. Each inclusion body pellet was resuspended in 50 mL of wash buffer #1 (50 mM Tris, 100 mM NaCl, 100 mM EDTA, 0.5% (v/v) Triton X-100, pH 8.0), homogenized, and sonicated for 30 seconds at an amplitude of 35%. Each mixture was then centrifuged at 27,000 × g for 15 minutes and the supernatant was discarded. This wash was repeated twice. The two inclusion body pellets resulting from the third round of the initial wash were each resuspended in 50 mL of wash buffer #2 (50 mM Tris, 10 mM EDTA, pH 8.0), homogenized, and sonicated for 30 seconds at an amplitude of 35%. Each mixture was then centrifuged at 15,000 × g for 15 minutes. This wash was repeated once. The two resulting washed inclusion body pellets were then completely unfolded by resuspension in 10 mL of a 7.12 M guanidine hydrochloride solution. This mixture was stirred at room temperature for 1 hour, and subsequently centrifuged at 12.000 × g for 10 minutes. The supernatant was drawn into a syringe, which was loaded onto a syringe pump, and added at a rate of 30 mL/hours to 1 L of chilled PBS that was being stirred rapidly. This solution of refolded protein was stirred continuously overnight at 4° C. Insoluble protein was then pelleted by centrifugation at 7000 × g for 15 minutes and discarded. The supernatant containing the folded SA was filtered by using a 0.45-µm bottle-top filter. The resulting filtrate was stirred vigorously, and ammonium sulfate was slowly added to a concentration of 1.9 M to precipitate out protein impurities. After being stirred for 3 hours at 4° C., the precipitate was removed by centrifugation for 10 min at 7000 × g. The supernatant was then filtered by using a 0.45-µm bottle-top filter. The ammonium sulfate concentration of the resulting filtrate was brought up to a total concentration of 3.68 M and stirred for 3 hours at 4° C. to precipitate the SA. The SA precipitate was pelleted by centrifugation at 7000 × g for 20 minutes, and resuspended in 20 mL of Iminobiotin Affinity Chromatography (IBAC) binding buffer (50 mM Sodium Borate, 300 mM NaCl, pH 11.0). This SA solution was then passed through 5 mL of Pierce iminobiotin Agarose (Thermo Scientific) in a gravity flow column (G-Biosciences) that had been pre-equilibrated with 5 column volumes of IBAC binding buffer. The IBAC column containing the bound SA was then washed with 20 column volumes of IBAC binding buffer. Then, 8 column volumes of elution buffer were passed through the column. The eluate was collected, dialyzed into PBS, and concentrated using a 10 kDa MWCO centrifugal filter (Millipore Sigma) SA was quantified by measuring the UV absorption at 280 nm.

### Assembly and Purification of MS2-SA VLPs

Biotinylated MS2 was added dropwise to a molar excess of concentrated SA solution that was stirred vigorously in a 5-mL glass vial. After a 30-minute incubation, the MS2-SA VLP was separated from the excess SA through SEC with a Superdex 200 Increase 10/300 column (Cytiva). The MS2-SA VLP was quantified by boiling a small aliquot at 90° C. in Nu-PAGE lithium dodecyl sulfate (LDS) sample buffer (invitrogen) for 30 minutes and running the sample on a polyacrylamide gel. SA standards with known concentrations quantified by UV absorption at 280 nm were also run on the gel. Comparing the intensities of the bands resulting from the SA standards with the intensity of the band representing the SA from the MS2-SA allowed for quantification of the VLP

### Expression and Purification of SARS-CoV-2 S Proteins

DNA encoding the S-2P (Wrapp et al., 2020) and HexaPro (Hsieh et al., 2020) prefusion-stabilized versions of the SARS-CoV-2 S ectodomain (residues 1-1208) with a C-terminal T4 fibritin trimerization motif, AviTag, and a his-tag were cloned into pcDNA3.1 between the Ncol and Xhol restriction sites by Gene Universal Inc. (Newark, DE). These plasmids were transfected into Expi293F cells (Thermo Fisher Scientific) using the ExpiFectamine Transfection Kit and protocol (Thermo Fisher Scientific). Five days after transfection, the cells were pelleted by centrifugation for 20 minutes at 5500 × g. The supernatant was dialyzed into PBS and passed through 1 mL of HisPur Ni-NTA resin (Thermo Fisher Scientific) in a gravity flow column (G-Biosciences). The column was then washed with 40 mL of wash buffer (42 mM sodium bicarbonate. 8 mM sodium carbonate, 300 mM NaCl. 20 mM imidazole). The S proteins were eluted from the column by incubating the Ni-NTA resin with 3 mL of elution buffer (42 mM sodium bicarbonate, 8 mM sodium carbonate, 300 mM NaCl, 300 mM imidazole) for 5 minutes before allowing for flow by gravity. This elution procedure was repeated twice, resulting in 9 mL of eluate. The eluate was concentrated by using a 10-kDa MWCO centrifugal filter (Millipore Sigma). S proteins were buffer exchanged into 20 mM Tris, 20 mM NaCl, pH 8.0 to allow for in vitro biotinylation and were quantified by using the BCA assay (Thermo Scientific).

### In Vitro Biotinylation of AviTagued MS2 and SARS-CoV-2 S

Biotinylation was performed in vitro using a BirA biotin-protein ligase standard reaction kit (Avidity) following the manufacturer’s protocol. In brief, the protein solution (either MS2 or SARS-CoV-2 S) was buffer exchanged into a 20 mM Tris, 20 mM NaCl, pH 8.0 buffer and the protein concentration was adjusted to 45 µM. BirA and a proprietary mixture containing biotin, ATP, and magnesium acetate (Biomix B) was added to the protein solution. This solution was shaken vigorously at 37° C. After 2 hours at 37° C., more Biomix B was added, and the solution was nutated at 4° C. overnight. The proteins of interest were then purified through SEC with a Superdex 200 Increase 10/300 column (Cytiva) connected to an ÄKTA pure (Cytiva) and controlled by Unicorn 7.2 software (Cytiva). Biotinylated S proteins were quantified by using the BCA assay (Thermo Scientific).

### Expression and Purification of CR3022 and ACE2-Fc

The variable regions of the heavy and light chains of CR3022 (ter Meulen et al., 2006) were cloned into the TGEX-HC and TGEX-LC vectors (Antibody Design Labs), respectively, according to the manufacturer’s protocol. Likewise, ACE2 (residues 1-615) was cloned into TGEX-HC. The DNA was then transfected into Expi293F cells (Thermo Fisher Scientific) by using the ExpiFectamine Transfection Kit (Thermo Fisher Scientific) following the provided protocol, and the cells were incubated in a humidified incubator at 37° C. and 8% CO₂ for 5 days The cells were then centrifuged at 5500 × g for 20 minutes. The supernatant media was diluted twofold in PBS and run through a 1-mL MabSelect SuRe column (Cytiva) connected to an ÄKTA start (Cytiva) and controlled by Unicorn start 1.0 software (Cytiva) according to the manufacturer’s operation manual to purify the proteins. CR3022 and ACE2-Fc were quantified by using the BCA assay (Thermo Scientific)

### Sds-page

Protein samples were diluted fourfold in Nu-PAGE lithium dodecyl sulfate (LDS) sample buffer (Invitrogen). The samples were then boiled at 90° C. for 30 minutes. PageRuler Plus Prestained Protein Ladder (Thermo Scientific) and protein samples were pipetted into the wells of a 4-12% Bis-Tris gel (Invitrogen), which was run in MES-SDS buffer at 4° C. for 1 hour at 110 V. The gel was stained with SimplyBlue SafeStain (Invitrogen) and subsequently de-stained. Once sufficiently de-stained, the gel was imaged by using the ChemiDoc MP imaging system and Image Lab 5.2.1 software (Bio-Rad).

### Preparation of VLP-S

MS2-SA and biotinylated S protein were mixed in a stoichiometric ratio found by using analytical SEC. For example, molar ratios of 5:1, 4:1, 3:1, 2.5:1, 2:1, 1.5:1, 1:1, 1:1.5, 1:2, 1:2.5. 1:3. 1:4 or 1:5 may be employed. Analytical SEC was used to characterize mixtures consisting of 5 µg of biotinylated S protein and varying amounts of MS2-SA VLP. The ratio of the mixture that contained the least MS2-SA VLP and also did not have excess S protein appear on the chromatogram was the stoichiometric ratio used to create the VLP-S. The concentration of the VLP-S was adjusted such that the solution contained 0.12 µg of S per µL. The VLP-S were further characterized by use of ELISA. SEC, and DLS as described below.

### Characterization of S and VLP-S by ELISA

VLP-S and S protein in PBS were coated onto a Nunc Maxisorp 96-well plate such that each well contained 0.1 µg of S protein in 100 µL. After 1 hour, the protein solutions were discarded from the wells and each well was blocked with 200 µL of 5% BSA (EMD Millipore) in PBST (PBS with 0.05% Tween-20) for 45 minutes. The plate was then washed twice with PBST, and CR3022 and ACE2-Fc in 1% BSA in PBST were added to the appropriate wells such that each well contained either one CR3022 or ACE2-Fc molecule per S trimer. One hour later, the wells were washed twice with PBST and a horseradish peroxidase-conjugated anti-human IgG Fc fragment goat antibody (MP Biomedicals; 1:5000 dilution) in 1% BSA in PBST was added to each well and left to incubate for 1 hour. Then, the plate was washed twice with PBST and developed with TMB substrate solution (Thermo Scientific) for 3 minutes; the reaction was then stopped with 0.16 M sulfuric acid. The absorbance of each well at 450 nm was read using a Spectramax i3x plate reader (Molecular Devices) and Gen5 2.07 software (BioTek).

### Analytical SEC

A Superdex 200 increase 10/300 column (Cytiva) connected to an AKTA pure (Cytiva) and controlled by Unicorn 7.2 software (Cytiva) was equilibrated with PBS. The 1-mL sample loop was washed with PBS and then 950 µl of either VLP-S solution or S alone was loaded into the sample loop. Each sample included 5 µg of S protein. The sample loop was then flushed with PBS such that the sample was directed through the column at a flowrate of 0.5 mL/minutes. One column volume of PBS was run through the column. Unicorn 7 (Cytiva) was used to control the system and to output a chromatogram of UV absorbance at 210 nm.

### Dynamic Light Scattering

A UVette (Eppendorf) containing 100 µL of VLP-S at a concentration of ~0.05 µg S per µL was loaded into a DynaPro NanoStar Dynamic Light Scattering detector (Wyatt Technology). For each measurement, Dynamics software (Wyatt Technology) was used to allow the temperature to equilibrate to 25° C. to collect ten acquisitions, and to output the results. Results were displayed by % Mass using the Isotropic Spheres model.

### Negative Stain Transmission Electron Microscopy

Conventional negative-stain transmission electron microscopy (TEM) was performed, as described previously (Booth et al., 2011). Briefly. 4 µl of the diluted samples was applied onto glow-discharged 300 mesh copper grids (CF300-Cu; Electron Microscopy Sciences, PA), washed with PBS (1X), and stained in droplets of 1% phosphotungstic acid (PTA, PH 6-7) for 1 minute The grids were then drained from the grid backside and air-dried inside a petri dish for at least 30 minutes at room temperature to minimize the negative-stain artifacts of flattening and stacking (Jung & Mun, 2018).

### Plunge Freezing and Cryo-electron Microscopy

4 µl of the VLP suspension was added to a glow discharged copper grid (C-Flat 1.2/1.3. 400 mesh, Protochips). Grids were plunged frozen into liquid ethane by double-sided blotting using Vitrobot Mark IV (ThermoScientific) and stored in liquid nitrogen until imaging. Cryo-electron microscopy (cryo-EM) and cryo-electron tomography (cryo-ET) were performed as described previously on a Titan Knos (ThermoScientific Hillsboro, OR, USA) at 300 kV (Yang et al., 2021). Images (defocus of -5 µm) were recorded on a post-GIF Gatan K3 camera in EFTEM mode (4.603 Å/pixel) with a 20-eV slit, CDS counting mode, using SenalEM 3.8 (Mastronarde. 2005). A total dose of 25-30 e/ Å² was used and 34 frames were saved (1.14 e/ Å2 per frame). Frames were motion-corrected in MotionCor2 (Zheng et al., 2017). Images were low pass filtered to 10 Å for better visualization and contrast in EMAN2.2 (Galaz-Montoya et al., 2015).

### Virus and Titration Assays

The virus isolate SARS-CoV-2/UT-NCGM02/Human/2020/Tokyo was used in this study and was previously characterized in Syrian hamsters (Imal et al., 2020). Virus titrations were performed on Vero E6/TMPRSS2 cells that were obtained from the National Institute of infectious Diseases, Japan (Matsuyama et al., 2020). Cells were maintained in Dulbecco’s modified Eagle’s medium (DMEM) containing 10% fetal bovine serum (FBS) and antibiotic/antimycotic solution along with G418 (1 mg/ml).

To determine virus titers, confluent Vero E6/TMPRSS2 cells were infected with 100 µl of undiluted or 10-fold dilutions (10⁻¹ to 10⁻⁵) of clarified lung or nasal turbinate homogenates. After a 30-minute incubation, the inoculum was removed, the cells were washed once, and then overlaid with 1 % methylcellulose solution in DMEM with 5% FBS. The plates were incubated for three days, and then the cells were fixed and stained with 20% methanol and crystal violet to count the plaques.

### Hamster Immunization Study

Golden Syrian hamsters (4-week-old females) were immunized with either 60 µg of SARS-CoV-2 S protein presented on the MS2-SA VLP, an equal amount of MS2-SA VLP without the S protein, or an equal volume of sterile phosphate-buffered saline (PBS) by subcutaneous inoculation Alhydrogel (2% solution; InvivoGen) added at an equal volume was thoroughly mixed with each vaccine preparation before inoculation. Animals were infected by intranasal inoculation with 10³ plaque-forming units (PFU) of SARS-CoV-2 while under isoflurane anesthesia. Animals were monitored daily for signs of illness and their body weights were recorded daily. Three days after infection, the animals were humanely sacrificed, and lung tissue and nasal turbinate samples were collected.

Serum was isolated from blood samples collected via the sublingual vein before the immunization and challenge with virus.

Detection of antibodies against the RBD of SARS-CoV-2 S in immunized hamsters by ELISA. The ELISA was performed using a recombinant SARS-CoV-2 S RBD protein produced in Expi293F cells (Thermo Fisher Scientific) and then C-terminal his-tag purified by using TALON metal affinity resin. ELISA plates were coated overnight at 4° C. with 50 µl of the RBD protein at a concentration of 2 µg/ml in PBS. After being blocked with PBS containing 0.1% Tween 20 (PBS-T) and 3% milk powder, the plates with incubated in duplicate with heat-inactivated serum diluted in PBS-T with 1% milk powder. Goat anti-hamster IgG secondary antibody conjugated with horseradish peroxidase (Invitrogen; 1:7000 dilution) was used for detection. Plates were developed with SigmaFast o-phenylenediamine dihydrochloride solution (Sigma), and the reaction was stopped with the addition of 3 M hydrochloric acid. The absorbance was measured at a wavelength of 490 nm (OD490). Background absorbance measurements from serum collected before immunization were subtracted from the absorbance measurements from plasma collected before challenge for each dilution. IgG antibody endpoint titers were defined as the highest plasma dilution with an OD₄₉₀ cut-off value of 0.15.

### Neutralization Assay

Virus (~100 PFU) was incubated with the same volume of two-fold dilutions of heat-inactivated serum for 30 minutes at 37° C. The antibody/virus mixture was added to confluent Vero E6/TMPRSS2 cells that were plated at 30,000 cells per well the day prior in 96-well plates. The cells were incubated for 3 days at 37° C. and then fixed and stained with 20% methanol and crystal violet solution. Virus neutralization titers were determined as the reciprocal of the highest serum dilution that completely prevented cytopathic effects.

### Statistics and Reproducibility

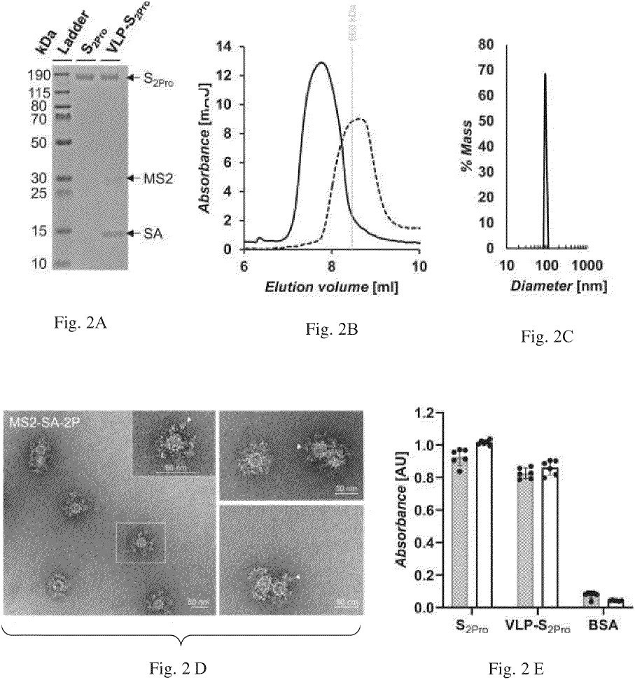

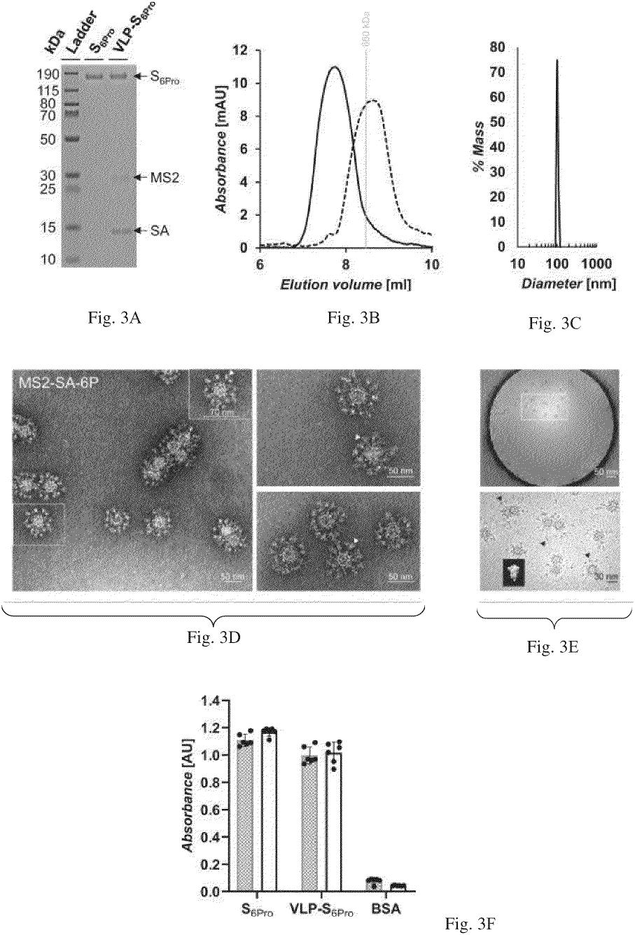

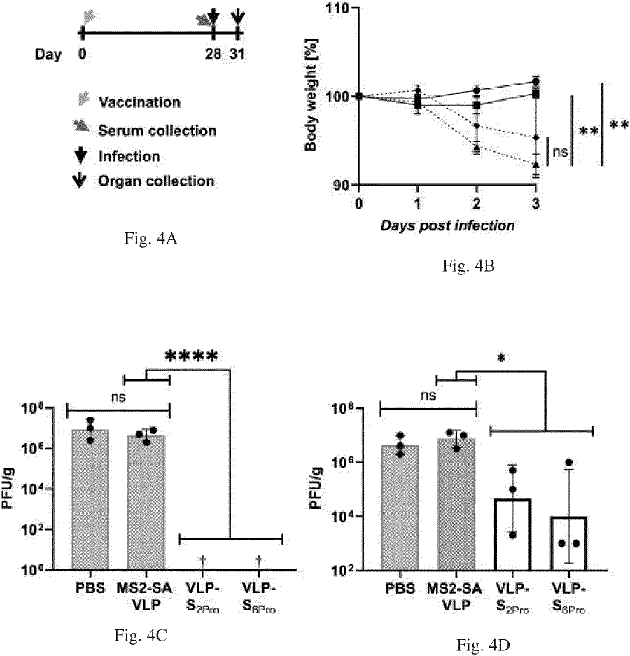

In vitro characterizations of the binding of Fc-ACE2 and CR3022 to the VLP-S constructs using ELISA (FIGS. 2e and 3f) were each conducted twice independently with three technical replicates for each condition. The data are presented as the mean 1 SD. For in vivo characterization, there were four groups (receiving either VLP-S, VLP-S. MS2-SA, or PBS) each with three hamsters (n = 3). To determine the resulting RBD IgG Endpoint Titers and Neutralizing Antibody titers (Table 2): three independent assays were conducted using sera from each hamster. The data are presented for each independent assay and also as the geometric mean with the geometric SD factor. Bodyweight after challenge with SARS-CoV-2 (FIG. 4b) was presented as the mean ± SD and significance was determined by a one-way analysis of variance (ANOVA) and Dunnett post-hoc multiple comparison between groups (α = 0.1). Assumptions of the normality of residuals and homogeneity of variance were validated by the D′Agostino-Pearson test and the Brown-Forsythe test, respectively. Viral titers in the lungs and nasal turbinates of hamsters immunized with either PBS, MS2-SA VLP, VLP-S_{2Pro} or VLP-S_{6Pro} 3 days after SARS-CoV-2 infection (FIGS. 4c, d) were presented as the geometric mean with geometric SD (n = 3) and significance was determined by a one-way analysis of variance (ANOVA) and Dunnett post-hoc multiple comparison between groups (α = 0.1). Assumptions of the normality of residuals and homogeneity of variance were validated by the Shapiro-Wilk test and the Brown-Forsythe test, respectively. All statistical analysis was carried out using Excel 2013 (Microsoft) and Prism 8 (GraphPad).

### MS2-AviTag Sequence

MASNFTQFVLVDNGGTGDVTVAPSNFANGVAEWISSNSRSQAYKVTCSVR
QSSAQNRKYTIKVEVPKVATQTVGGVELPVAAWRSYLNMELTIPIFATNS
DCELIVKAMQGLLKDGNPIPSAIAANSGIYASNFTQFVLVDNGGGLNDIF
EAQKIEWHETGDVTVAPSNFANGVAEWISSNSRSQAYKVTCSVRQSSAQN
RKYTIKVEVPKVATQTVGGVELPVAAVASYLNMELTIPIFATNSDCELIV
KAMQGLLKDGNPIPSAIAANSGIY

(SEQ ID NO:1), or a polypeptide with at least 80%, 85%, 90%, 92%, 94%, 95%, 97%, 98% or 99% amino acid sequence identity thereto.

### S_{2Pro} Sequence

MFVFLVLLPLVSSQCVNLTTRTQLPPAYTNSFTRGVYYPDKVFRSSVLHS
TQDLFLPFFSNVTWFHAlHVSGTNGTKRFDNPVLPFNDGVYFASTEKSNI
IRGWIFGTTLDSKTQSLLIVNNATNVVIKVCEFQFCNDPFLGVYYHKNNK
SWMESEFRVYSSANNCTFEYVSQPFLMDLEGKQGNFKNLREFVFKNIDGY
FKIYSKHTPINLVRDLPQGFSA LEPLVDLPIGINITRFQTLLALHRSYL
TPGDSSSGWTAGAAAYYVGYLQPRTFLLKYNENGTITDAVDCALDPLSET
KCTLKSFTVEKGIYQTSNFRVQPTESIVRFPNITNLCPFGEVFNATRFAS
VYAVVNRKRISNCVADYSVLYNSASFSTFKCYGVSPTKLNDLCFTNVYAD
SFVIRGDEVRQIAPGQTGKIADYNYKLPDDFTGCVIAVVNSNNLDSKVGG
NYNYLYRLFRKSNLKPFERDISTEIYQAGSTPCNGVEGFNCYFPLQSYGF
QPTNGVGYQPYRVVVLSFELL HAPATVCGPKKSTNLVKNKCVNFNFNGL
TGTGVLTESNKKFLPFQQFGRDIADTTDAVRDPQTLEILDITPCSFGGVS
VITPGTNTSNQVAVLYQDVNCTEVPVAIHADQLTPTWRVYSTGSNVFQTR
AGCLIGAEHVNNSYECDIPIGAGICASYQTQTNSPGSASSVASQSIIAYT
MSLGAENSVAYSNNSIAIPTNFTISVTTEILPVSMTKTSVDCTMYICGDS
TECSNLLLQYGSFCTQLNRALTGIAVEQDKNTQEVFAQVKQIYKTPPIKD
FGGFNFSQILPDPSKPSKRSFIEDLLFNKVTLADAGFIKQYGDCLGDIAA
RDLICAQKFNGLTVLPPLLTDEMIAQYTSALLAGTITSGWTFGAGAALQI
PFAMQMAYRFNGIGVTQNVLYENQKLIANQFNSAIGKIQDSLSSTASALG
KLQDVVNQNAQALNTLVKQLSSNFGAISSVLNDILSRLDPPEAEVQIDRL
ITGRLQSLQTYVTQQLIRAAEIRASANLAATKMSECVLGQSKRVDFCGKG
YHLMSFPQSAPHGVVFLHVTYVPAQEKNFTTAPAICHDGKAHFPREGVFV
SNGTHWFVTQRNFY EPQIITTDNTFVSGNCDVVIGIVNNTVYDPLQPEL
DSFKEELDKYFKNHTSPDVDLGDISGINASVVNIQKEIDRLNEVAKNLNE
SLIDLQELGKYEQGSGYIPEAPRDGQAYVRKDGEWVLLSTFLGGLNDIFE
AQKIEWHEHHHHHH

(SEQ ID NO:2), or a polypeptide with at least 80%, 85%, 90%, 92%, 94%, 95%, 97%, 98% or 99% amino acid sequence identity thereto.

### S_{6Pro} Sequence

MFVFLVLLPLVSSQCVNLTTRTQLPPAYTNSFTRGVYYPDKVFRSSVLHS
TQDLFLPFFSNVTWFHAIHVSGTNGTKRFDNPVLPFNDGVYFASTEKSNI
IRGWIFGTTLDSKTQSLLIVNNATNVVIKVCEFQFCNDPFLGVYYHKNNK
SWMESEFRVYSSANNCTFEYVSQPFLMDLEGKQGNFKNLREFVFKNIDGY
FKIYSKHTPINLVRDLPQGFSA LEPLVDLPIGINITRFQTLLALHRSYL
TPGDSSSGWTAGAAAYYVGYLQPRTFLLKYNENGTITDAVDCALDPLSET
KCTLKSFTVEKGIYQTSNFRVQPTESIVRFPNITNLCPFGEVFNATRFAS
VYAWNRKRISNCVADYSVLYNSASFSTFKCYGVSPTKLNDLCFTNVYADS
FVIRGDEVRQIAPGQTGKIADYNYKLPDDFTGCVIAWNSNNLDSKVGGNY
NYLYRLFRKSNLKPFERDISTEIYQAGSTPCNGVEGFNCYFPLQSYGFQP
TNGVGYQPYRVVVLSFELL HAPATVCGPKKSTNLVKNKCVNFNFNGLTG
TGVLTESNKKFLPFQQFGRDIADTTDAVRDPQTLEILDITPCSFGGVSVI
TPGTNTSNQVAVLYQDVNCTEVPVAIHADQLTPTVVRVYSTGSNVFQTRA
GCLIGAEHVNNSYECDIPIGAGICASYQTQTNSPGSASSVASQSIIAYTM
SLGAENSVAYSNNSIAIPTNFTISVTTEILPVSMTKTSVDCTMYICGDST
ECSNLLLQYGSFCTQLNRALTGIAVEQDKNTQEVFAQVKQIYKTPPIKDF
GGFNFSQILPDPSKPSKRSPIEDLLFNKVTLADAGFIKQYGDCLGDIAAR
DLICAQKFNGLTVLPPLLTDEMIAQYTSALLAGTITSGWTFGAGPALQIP
FPMQMAYRFNGIGVTQNVLYENQKLIANQFNSAIGKIQDSLSSTPSALGK
LQDVVNQNAQALNTLVKQLSSNFGAISSVLNDILSRLDPPEAEVQIDRLI
TGRLQSLQTYVTQQLIRAAEIRASANLAATKMSECVLGQSKRVDFCGKGY
HLMSFPQSAPHGVVFLHVTYVPAQEKNFTTAPAICHDGKAHFPREGVFVS
NGTHWFVTQRNFY EPQIITTDNTFVSGNCDVVIGIVNNTVYDPLQPELD
SFKEELDKYFKNHTSPDVDLGDISGINASVVNIQKEIDRLNEVAKNLNES
LIDLQELGKYEQGSGYIPEAPRDGQAYVRKDGEWVLLSTFLGGLNDIFEA
QKIEWHEHHHHHH

(SEQ ID NO:3), or a polypeptide with at least 80%, 85%, 90%, 92%, 94%, 95%, 97%, 98% or 99% amino acid sequence identity thereto.

### Results and Discussion

### Generation and in Vitro Characterization of Nanoparticle-based Vaccines

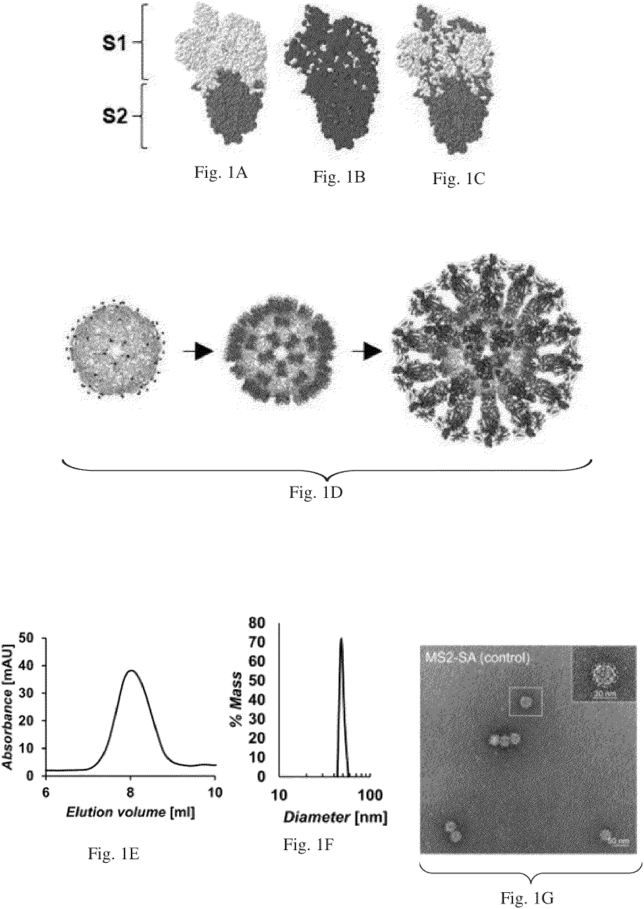

A general platform for the VLP-based multivalent display of the S protein of SARS-CoV-2 was developed. VLPs comprise coat proteins that self-assemble to form repetitive, dense arrays of antigen that emulate the size and geometry of natural viruses (Bachmann & Jennings, 2010). We generated VLPs coated with streptavidin (SA) that display biotinylated antigens, such as biotinylated SARS-CoV-2 S protein (FIG. 1a), based on the very high-affinity biotin-streptavidin interaction.

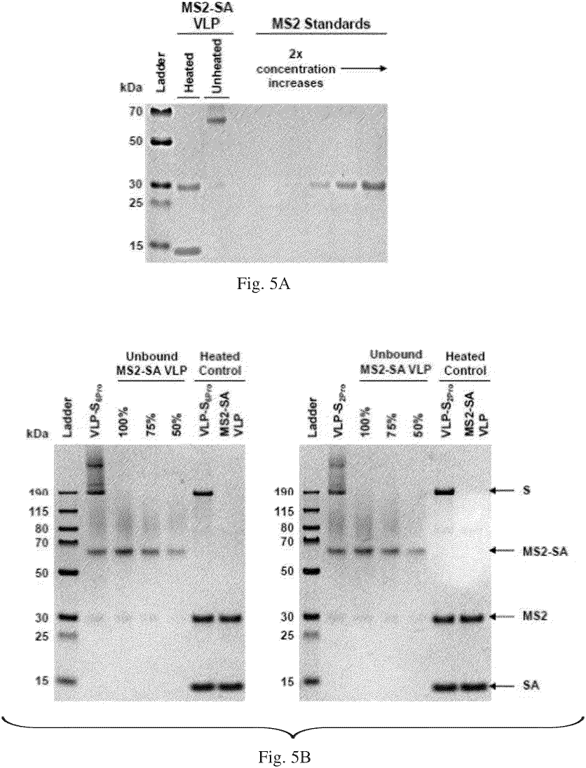

Specifically, VLPs were generated based on the coat protein of the RNA bacteriophage MS2 (Frietze et al., 2016; Valegard et al., 1994). MS2 consists of 180 monomeric coat proteins that self-assemble to form an icosahedral structure consisting of 90 homodimers. Peabody et al. generated a variant of the MS2 coat protein in which the two subunits of the dimer were genetically fused and found that a surface loop on this single-chain dimer could tolerate the insertion of a peptide (Peabody et al., 2008). Accordingly, a single-chain MS2 coat protein dimer was generated wherein the second monomer had an AviTag inserted in this surface loop (see sequences above). The inserted AviTag allows for site-specific biotinylation by the enzyme BirA. DNA encoding this MS2-AviTag construct was co-expressed with BirA in BL21(DE3) competent Escherichia coli (E. coli) cells. Following expression, the cells were lysed and the MS2-AviTag was purified by using HiScreen Capto Core 700 columns and size exclusion chromatography (SEC). The purified MS2-AviTag was partially biotinylated due to its co-expression with BirA. A commercially available kit was then used to further biotinylate the MS2-AviTag in vitro, which resulted in near 100% biotinylation. The MS2-Biolin was then added dropwise to an excess of SA, which had been expressed as inclusion bodies, refolded, and purified using Iminobiotin Affinity Chromatography (IBAC). The resulting MS2-SA VLPs were separated from the excess SA through SEC. Sodium dodecyl sulfate-polyacrylamide gel electrophoresis (SDS-PAGE) was used to determine that each purified MS2-SA VLP contained ~72 streptavidin molecules (FIG. 5). The purified MS2-SA VLPs were further characterized by analytical SEC (FIG. 1b), dynamic light scattering (DLS) (FIG. 1c), and negative-stain transmission electron microscopy (NS-TEM) (FIG. 1d). Characterization by DLS indicated that the purified MS2-SA VLPs were ~50 nm in diameter, whereas characterization by NS-TEM indicated uniform particles with a diameter of ~30 nm. The larger average size indicated by DLS may arise because the scattering intensity is proportional to the sixth power of the radius, resulting in a disproportionately higher weighting to larger particles.

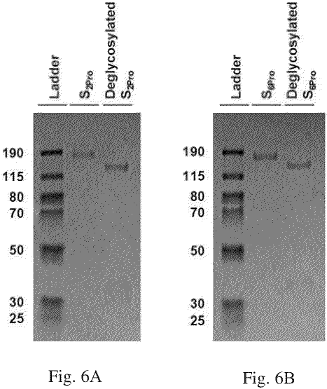

A biotinylated variant of the SARS-CoV-2 S protein was generated that could be displayed on the MS2-SA VLPs. Wrapp et al. recently reported a prefusion-stabilized variant of the SARS-CoV-2 S protein, S-2P, which contains 2 proline substitutions (Wrapp et al., 2020). To make a version of this variant that was compatible with display on the MS2-SA VLPs, plasmids were created encoding the stabilized prefusion S ectodomain with a C-terminal AviTag and a his-tag, which is termed S_{2Pro} (see sequences). The AviTag allows biotinylation and subsequent conjugation to the VLPs, whereas the his-tag allows purification by use of immobilized metal affinity chromatography (IMAC). The S_{2Pro} protein was expressed in Expi293F cells and the secreted protein purified from the cell culture media by using IMAC. The protein was then biotinylated enzymatically in vitro by BirA. Finally, the protein was separated from BirA and other impurities by using SEC and the purity was characterized by use of SDS-PAGE (FIG. 2a and FIG. 6).

The punfied, biotinylated S_{2Pro} protein was then mixed with the MS2-SA VLPs to form VLP-S_{2Pro}. SDS-PAGE was used to determine that each purified VLP-S_{2Pro} particle contained ~18 S_{2Pro} molecules (FIG. 5). Further SDS-PAGE analysis of the VLP-S_{2Pro} revealed the expected three distinct bands (FIG. 2a): the upper band runs alongside S protein alone and appears at ~140 kDa, which corresponds to the approximate molecular weight of a single monomer of the S trimer; the middle band appears at the molecular weight of an MS2 coat protein dimer (~29 kDa); and the lower band corresponds to the molecular weight of a monomer of SA (~14 kDa). This characterization indicates that the VLP-S_{2Pro} is pure and consists of only S_{2Pro}, SA, and MS2. The VLP-S_{2Pro} construct was also characterized by using analytical SEC (FIG. 2b). The UV trace of the VLP-S_{2Pro} is represented by a solid line, which appears as a single peak with no trailing shoulder. The lack of a trailing shoulder suggests that there is little to no unbound S_{2Pro} protein in the VLP-S_{2Pro} solution, as the UV trace of the S_{2Pro} protein alone results is a single peak that slightly trails the peak of the VLP-S and is represented by a dashed line. Furthermore, the locations of the peaks are consistent with the constructs’ size relative to the size of the molecular weight standard thyroglobulin (660 kDa). The location at which thyroglobulin elutes is represented by a vertical gray line.

The VLP-S_{2Pro} constructs were characterized by DLS (FIG. 2c) and then by NS-TEM (FIG. 2d) to confirm the presence and coating efficiency of biotinylated S_{2Pro} on the VLP. Consistent with biochemical characterization, VLP-S_{2Pro} displayed clear three-component layers, from outside to inside, prefusion-stabilized variants of S_{2Pro}, SA, and MS2 (FIG. 2d). Compared to the naked MS2-SA, glycoprotein S_{2Pro} decorates the exterior of VLP-S_{2Pro} (FIG. 2d, white arrowheads), forming a ~20 nm layer of a spike-containing protein shell. This result is consistent with expectations, as the S protein (with the trimerization domain and C-terminal AviTag) would theoretically be ~20 nm in length. Finally, to ensure that the S proteins remained properly folded after conjugation to the VLPs, the binding of ACE2-Fc and the receptor-binding domain (RBD)-binding monoclonal antibody CR3022 to S_{2Pro} protein alone and to VLP-S_{2Pro} (FIG. 2e) was assessed ACE2 is the cellular receptor for SARS-CoV-2 and binds to the receptor-binding motif of the S protein (Lan et al, 2020). A common mechanism of SARS-CoV-2 neutralization is the inhibition of S protein binding to ACE2, so it is important to demonstrate that the ACE2 binding site is properly folded (Ju et al.. Shi et al., 2020). CR3022 is an antibody that binds to the S protein RBD outside of the ACE2 binding site (Ju et al, 2020; ter Meulen et al., 20060. ELISA showed that both ACE2-Fc and CR3022 can bind to the S_{2Pro} protein alone and to VLP-S_{Pro} This analysis demonstrates that the protein epitopes needed to elicit a neutralizing immune response to SARS-CoV-2 are correctly folded and accessible.

VLPs displaying multiple copies of a second prefusion-stabilized variant of the S protein were generated, called HexaPro, which was reported by Hsieh et al. to be more stable than S-2P and give a higher expression yield (Hsieh 2020). A variant of HexaPro containing a C-terminal AviTag and a his-tag, which is termed S_{aPro} (FIG. 3a), was expressed (see sequences above). VLP-S_{6Pro} were generated and characterized (FIG. 3 and FIG. 5) as described above for VLP-S_{2Pro}. In addition, to preserve the sample’s native integrity, minimize conformational changes possibly introduced during the negative stain process, and further confirm the incorporation of spike proteins, cryo-electron microscopy (cryo-EM) was performed on the VLP-S_{6Pro} constructs. The MS-SA core was an approximately icosahedral sphere 30 nm in diameter and S_{6Pro} spikes were studded on the core and formed the outer shell (FIG. 3e), the morphology of which was comparable to the previous reported structure of S_{6Pro} (EMD: 22221 (Hsieh et al., 2020)).

### Protective Efficacy and Antibody Response to a Single Immunization in Syrian Hamsters

The antibody responses elicited by these nanoparticle-based vaccine candidates were assessed in Syrian hamsters. Syrian hamsters are highly susceptible to SARS-CoV-2 infection and present with pathological phenotypes similar to those of infected humans, making hamsters an ideal animal model to evaluate vaccine candidates (Imal et al., 2020; Sia et al.. 2020). Hamsters (four groups; three anifrials/group) were immunized with VLP-S_{2Pro}, VLP-S_{6Pro}, MS2-SA VLPs alone, or PBS along with Alhydrogel, an aluminum hydroxide base adjuvant. The hamsters were bled 28 days after immunization to characterize their antibody responses (FIG. 4a). Hamsters immunized once with the VLP-S conjugates had appreciable levels of IgG antibodies against the RBD of the S protein as determined by ELISA, with endpoint titers ranging from 2.6 × 10⁴ to 8.2 × 10⁴ and high neutralizing antibody titers (representing the reciprocal of the highest dilution that completely prevented cytopathic effects) ranging from 320 to 640 (Table 1). In contrast, as expected, negligible anti-S antibodies were detected in hamsters immunized with the controls (VLPs alone or PBS). We have compared these titers with some previously published reports. The assays used in the literature are not standardized and some reports have even used different animal models, and the differences must therefore be interpreted with caution. Tostanoski et al. (2020) characterized the immunogenicity of adenovirus serotype 26 (Ad 26) vector-based vaccines expressing a stabilized SARS-CoV-2 S protein in hamsters. Ad26-S.PP, also termed Ad26.COV2.S, which has been evaluated in clinical trials, showed median endpoint antibody titers against the S RBD of up to 4757 four weeks after the first immunization. While neutralization assays used a pseudovirus, the median neutralizing antibody half-maximal inhibitory concentration (IC₅₀) titers reported for the Ad26-S.PP were as high as 375. Corbett et al. (2020) conducted a study in mice to test the mRNA vaccine called mRNA-1273, for which they reported geometnc mean endpoint titers of 4479 (against S) 4 weeks after a single dose. Neutralizing antibody reciprocal IC₅₀ geometric mean titers against a pseudovirus 4 weeks after a single immunization with mRNA-1273 at the highest dose (10 µg) were 775. Thus, the present endpoint and neutralizing antibody titers compare well with those obtained using these other modalities that have been through clinical trials.

TABLE 2

Antibody responses to single immunization in Syrian hamsters.

| | | RBD IgG endpoint titer^{a} | | | | | Neutralizing antibody titer^{b} | | | | |
|---|---|---|---|---|---|---|---|---|---|---|---|
| Vaccine group | Animal # | Replicate | Replicate | Replicate | Geometric mean | Geometric SD factor | Replicate | Replicate | Replicate | Geometric mean | Geometric SD factor |
| | | 1 | 2 | 3 | | | 1 | 2 | 3 | | |
| PBS | 1 | <10 | <10 | <10 | | | <10 | <10 | <10 | | |
| | 2 | <10 | <10 | <10 | <10 | – | <10 | <10 | <10 | <10 | – |
| | 3 | <10 | <10 | <10 | | | <10 | <10 | <10 | | |
| MS2-SA VLP | 1 | <10 | <10 | <10 | | | <10 | <10 | <10 | | |
| | 2 | <10 | <10 | <10 | <10 | – | <10 | <10 | <10 | <10 | – |
| | 3 | <10 | <10 | <10 | | | <10 | <10 | <10 | | |
| VLP-S2_{Pro} | 1 | 20,480 | 40,960 | 20,480 | | | 320 | 640 | 320 | | |
| | 2 | 81,920 | 40,960 | 81,920 | 35,113 | 1.78 | 640 | 320 | 320 | 373 | 1.36 |
| | 3 | 20,480 | 20,480 | 40,960 | | | 320 | 320 | 320 | | |
| VLP-S_{6Pro} | 1 | 81,920 | 81,920 | 81,920 | | | 640 | 640 | 640 | | |
| | 2 | 81,920 | 81,920 | 81,920 | 70,225 | 1.36 | 640 | 640 | 640 | 549 | 1.36 |
| | 3 | 40,960 | 81,920 | 40,960 | | | 320 | 640 | 320 | | |

^{a}Viral antibody endpoint titers against the RBD (receptor-binding domain) from three independent assays (three animals in each group). Endpoint titers using 2-fold diluted sera were expressed as the reciprocal of the highest dilution with an optical density at 490 nm cutoff value >0.15; sera were collected on day 28 after immunization.
^{b}Viral neutralization titers from three independent assays (three animals in each group). Endpoint titers using twofold diluted sera were expressed as the reciprocal of the highest dilution that completely prevented cytopathic effects; sera were collected on day 28 after immunization.

Four weeks after immunization, the animals were intranasally inoculated with 10³ plaque-forming units of SARS-CoV-2/UT-NCGM02/Human/2020/Tokyo (Imal et al., 2020). While the animals in both control groups experienced significant weight loss, those immunized with VLP-S_{2Pro} had recovered their average initial weight by day 3, and those immunized with VLP-S_{6Pro} showed a slight increase in body weight over this 3-day period (FIG. 4b).

Three days after virus challenge, which is when virus levels in the lungs peak, the animals were sacrificed and lung and nasal turbinate samples were collected. As expected, animals in both control groups (PBS and MS2-SA VLPs) had high viral loads in the lungs; however, in hamsters immunized with VLP-S_{2Pro} or VLP-S_{6Pro} no infectious virus was detected in the lungs (FIG. 4c). The lack of infectious virus in the lungs was consistent with the differences observed in body weight change between the vaccine and control groups. Moreover, despite the intranasal mode of challenge with SARS-CoV-2, the hamsters immunized with VLP-S_{2Pro} or VLP-S_{6Pro} had less virus in their nasal turbinates (FIG. 4d), with mean titers more than 150-fold lower (VLP-S_{2Pro}) and more than 700-fold lower (VLP-S_{6Pro}) relative to MS2-SA VLP controls.

In summation, a highly effective nanoparticle-based vaccine was developed that provides protection in an animal model against SARS-CoV-2 after a single immunization. While these results are exciting, it is important to consider obstacles that might arise during clinical translation. One potential concern with subunit vaccines is the expression yield. Hsieh et al. designed the S_{aPro} variant by introducing substitutions that improve both expression yield and stability and reported a yield of 10.5 mg/L in FreeStyle 293-F cells (Hsieh et al., 2020). Expression in insect cells could also be evaluated for improving the expression yield. The concerns about antibody responses are mitigated in part by the protective efficacy after a single dose. However, if necessary, the scaffold could be further shielded from the immune system by using techniques such as nanopatterning (Arsiwala 2019). Another important issue is the emergence of “variants of concern”. In this context, vaccine platforms that generate a robust immune response would be advantageous as they might still be able to provide some protection against resistant strains despite a reduction in neutralizing antibody titers. Our vaccine platform could also be readily adapted for the display of S proteins from variant strains. In the future, it would be particularly important to use this platform to display engineered antigens that provide broader-pan.sarbecovirus or pan-coronavirus-immunity. Given the number of people that must be immunized, and societal habits, an ideal vaccine against SARS-CoV-2 would offer protection after only one immunization. The development of multiple effective vaccine platforms (Tostanoski et al., 2020; Jia et al., 2021; Sanchez-Felipe et al., that can offer such protection is important because vaccines remain the best approach for protection from current and future pandemics. The nanoparticle-based vaccine platform described here should be broadly applicable for protecting against important pathogens including: but not limited to, SARS-CoV-2.

### Example 3

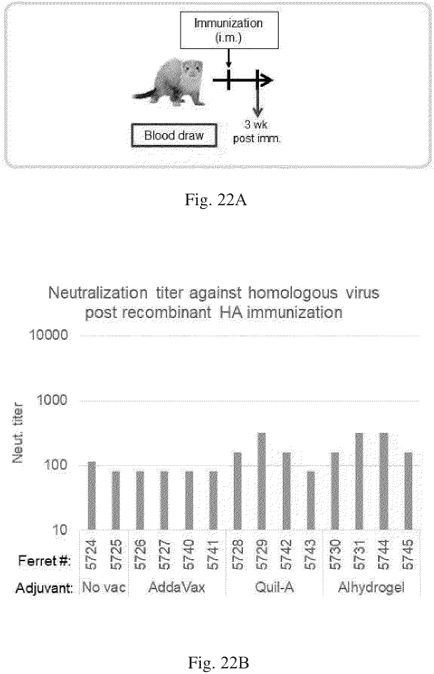

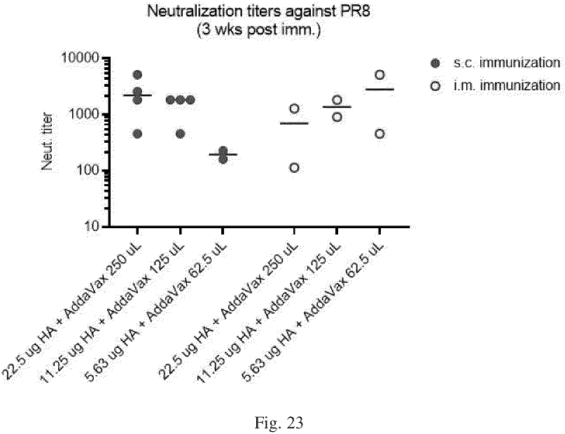

Coronavirus SARS-CoV-2 has had an astounding impact on world health since it was first identified in December 2019. In fact, over 3 million people worldwide have died as a result of contracting the virus, and many more have been infected. A wide variety of SARS-CoV-2 vaccine candidates are being developed, including nucleic acid-based vaccines, viral vector-based vaccines, subunit vaccines, and inactivated vaccines (Krammer, 2020), In particular, a number of vaccines that target the SARS-CoV-2 spike (S) protein have been authorized for use (Baden et al., 2021; Polack et al., 2020; Sadoff et al., 2021; Voysey et al., 2021). The spike protein is a glycoprotein displayed on the surface of the SARS-CoV-2 virus that allows the virus to bind to host cells through its S1 subunit and fuse to the host cell membrane through its S2 subunit (FIG. 18a). This key role in viral attachment and entry into cells has made the S protein an effective vaccine target and several S protein-based vaccines have been shown to successfully prevent SARS-CoV-2 infection (Baden et al., 2021; Polack et al., 2020; Sadoff et al., 2021; Voysey et al., 2021). However, the threat of emerging variants that may escape vaccine-mediated immunity is a cause for concern. In addition, some zoonotic coronaviruses have been identified to have pandemic potential (Menachery et al., 2015; Menachery et al., 2016) and others such as SARS-CoV-1 and MERS-CoV are already known 1 to cause severe disease in humans. Therefore, a broadly protective coronavirus vaccine may prove useful.

The S2 subunit of the spike protein has been identified as a promising target for a broadly protective coronavirus vaccine, as it is considerably more conserved than the S1 subunit. In particular, the functionally important fusion peptide region in the S2 subunit may be an attractive target for cross-reactive antibodies (Walls et al., 2020; Walls et al., 2016). Antibodies targeting the S2 subunit have been isolated from convalescent COVID-19 patients and found to neutralize SARS-CoV-2 (Chi et al., 2020; Song et al., 2020; Jennewein et al., 2021; Pinto et al., 2021; Zhou et al, 2021). Even S2-specific antibodies that do not directly neutralize SARS-CoV-2 may mitigate pathological burden through Fc effector functions (Shiakolas 2021). Furthermore, antibodies targeting the S2 subunit have been found to be cross-reactive among coronaviruses (Ladner et al., 2021; Nguyen-Content et al., 2020; Wang et al., 2021; Sauer et al., 2021). For instance, Wang et al. isolated two human monoclonal antibodies from immunized humanized mice that displayed cross-reactivity against the spike proteins of betacoronaviruses including SARS-CoV, SARS-CoV-2, MERS-CoV, and HCoV-OC43 Wang et al., 2021). Some cross-reactive S2-specific antibodies are also capable of neutralizing across coronavirus types (Song et al., 2020; Jennewein et al., 2021; Pinto et al., 2021; Zhou et al., 2021; sauer et al., 2021). For example, Pinto et al. (2021) described a human S2-specific monoclonal antibody that showed neutralization activity against not only authentic SARS-CoV-2 but also against viruses pseudotyped with SARS-CoV-1 S, Pangolin Guangdong 2019 S, MERS-CoV S, and OC43 S. Collectively, these findings indicate that S2-based vaccines may provide broad protection against coronaviruses.

Recently, an S2 immunogen was evaluated by Ravichandran et al. (2020). They found that compared to the spike ectodomain and other S1-based antigens, the S2 immunogen generated relatively low anti-spike antibody titers and weak SARS-CoV-2 neutralization titers. It was hypothesized that the multivalent display of the S2 subunit with an appended C-terminal trimerization domain, e.g., the generation of nanoparticle scaffolds presenting multiple copies of the stabilized S2 subunit trimers, to promote its stability might help elicit a strong response against S2, as the multivalent display of antigens has been shown to generate strong immune responses (Bachmann & Zinkemagel, 1997). Moreover, it was hypothesized that such an immunogen without the immunodominant S1 subunit would elicit a strong response targeting the S2 subunit that would have otherwise been directed towards the S1 subunit.

### Methods

### Expression and Purification of SARS-CoV-2 S2 and S2_{mutS2′} Proteins

The gene encoding the S2 subunit of the SARS-COV-2 HexaPro (Hsieh et al., 2020) spike protein (residues 686 to 1208) with an N-terminal mouse Ig Kappa signal peptide and C-terminal T4 fibritin trimerization domain, AviTag, and his-tag was cloned into pcDNA3.1 between the Ncol and Xhol restriction sites by Gene Universal, Inc. (Newark DE). The S2_{mutS2′} variant was created such that S2 residues 814 and 815 were mutated to glycine residues to eliminate the S2′ protease cut site. These plasmids were transfected into Expi293F cells using the ExpiFectamine Transfection Kit (Thermo Fisher Scientific) and associated protocol. The cells were incubated for 5 days, after which the cultures were centrifuged at 5,500xg for 20 minutes. The supernatant was dialyzed into PBS and then was allowed to flow through 1 mL of of HisPur Ni-NTA resin (Thermo Scientific) in a gravity flow column (G-Biosciences) that had been washed with DI water and pre-equilibrated with phosphate-buffered saline (PBS). The column was then washed with 90 column volumes of wash buffer (42 mM sodium bicarbonate, 8 mM sodium carbonate, 300 mM NaCl, 20 mM imidazole). The protein was eluted by incubating the resin 1 in 3 mL of elution buffer (42 mM sodium bicarbonate, 8 mM sodium carbonate, 300 mM NaCl, 300 mM imidazole) for 5 minutes before allowing the elution buffer to flow through the column. The eluate was collected. This elution procedure was repeated twice more such that a total of 9 mL of eluate was collected. The eluate was buffer exchanged into 20 mM Tris, 20 mM NaCl, pH 8.0, to prepare for in vitro biotinylation. The concentration of the protein solutions was quantified using the BCA assay (Thermo Scientific).

### Expression and Purification of MS2

The following protocol regarding the expression and purification of MS2 has been previously described (Chiba et al., 2020). The DNA sequence corresponding to a single chain dimer of MS2 coat protein with an AviTag inserted between the fourteenth and fifteenth residues of the first coat protein monomer was cloned into pET-28b between the Ndel and Xhol restriction sites by GenScript Biotech Corporation (Piscataway, NJ). This plasmid and a plasmid coding for pAcm-BirA (Avidity LLC) were co-transformed into BL21(DE3) Escherichia coli (E. coli) (New England BioLabs). The transformation was added to 5 mL of 2×YT that had been supplemented with kanamycin and chloramphenicol. This small culture was incubated in a shaking incubator overnight at 37° C. The following morning, the 5 mL culture was added to 1 L of 2×YT that had been supplemented with kanamycin and chloramphenicol. The 1 L culture was placed in a shaking incubator at 37° C. Once the culture’s optical density reached 0.6, expression of the MS2 and BirA was induced with IPTG (1 mM; GoldBio). The culture was also supplemented with approximately 12.5 µg of biotin, and remained shaking in the incubator overnight at 30° C. After the overnight expression, the culture was centrifuged at 7000×g for 7 minutes to pellet the cells. The cell pellet was then homogenized into 25 mL of 20 mM Tris buffer (pH 9.0) supplemented with lysozyme (0.5 mg/mL; Alfa Aesar), a protease inhibitor tablet (Sigma-Aldrich), and benzonase (125 units; EMD Millipore). The resulting cell suspension was kept on ice for 20 minutes while occasionally mixing. Next, sodium deoxycholate was added to a final concentration of 0.1% (w/v). The cells were kept on ice and sonicated for 3 minutes at an amplitude of 35% with 3 second pulses (Sonifier S-450, Branson Ultrasonics). This sonication procedure was repeated after allowing the cells to cool on ice for at least 2 minutes. The resulting lysate was centrifuged at 27,000×g for 30 minutes. The supernatant was collected and was centrifuged again at 12,000×g for 15 minutes. The supernatant resulting from the second centrifugation was diluted 3-fold with 20 mM Tris, pH 8.0, and filtered using a 0.45 µm bottle-top filter. The filtrate was then run through four HiScreen Capto Core 700 columns (Cytiva) in parallel according to the manufacturer’s operating instructions, resulting in fractions that contained MS2. The fractions were run on an SDS-PAGE gel to assess MS2 purity and recovery. Fractions containing pure MS2 were pooled, concentrated using a 10 kDa MWCO centrifugal filter (Millipore Sigma), and further purified using a Superdex 200 increase 10/300 SEC column (Cytiva). The SEC fractions containing MS2 were pooled and buffer exchanged into 20 mM Tris, 20 mM NaCl, pH 8.0, in preparation for in vitro biotinylation. MS2 was quantified using the BCA assay (Thermo Scientific).

### Expression, Refolding, and Purification of Streptavidin (SA)

The following protocol regarding the expression, refolding, and purification of SA has been previously described and was adapted from methods documented by Fairhead et al. and Howarth et al. (Chiba et al., 2020; Fairhead et al., 2014; Howarth & Ting, (2008). A plasmid encoding SA (Addgene plasmid #46367) was transformed into BL21(DE3) E. coli. The transformation was added to 5 mL of 2×YT supplemented with ampicillin, and this small culture was grown overnight in a shaking incubator at 37° C. The next morning the culture was added to four, 1 L shake flasks of 2xYT supplemented with ampicillin. These larger cultures were placed in a shaking incubator at 37° C. until the cultures’ OD reached 0.6, at which point the expression of streptavidin as inclusion bodies was induced with IPTG (1 mM; GoldBio), and the temperature of the incubator was reduced to 30° C. After overnight incubation, the cultures were centrifuged at 7,000×g for 15 minutes such that there were two cell pellets. The two resulting cell pellets were each homogenized into 50 mL of resuspension buffer (50 mM Tris, 100 mM NaCl, pH 8.0) supplemented with lysozyme (1 mg/mL; Alfa Aesar) and benzonase (500 units; EMD Millipore). The homogenized cells were incubated at 4° C. for at least 30 minutes. After this incubation step, the cells were further homogenized and sodium deoxycholate was added to a final concentration of 0.1% (w/v) before sonicating (Sonifier S-450, Branson Ultrasonics) for 3 minutes with 3 second pulses at 35% amplitude. The lysed cells were then centrifuged at 27,000xg for 15 minutes. The supernatant was discarded, and the lysis procedure was repeated. When the lysis step was repeated the incubation time at 4° C. prior to sonication was reduced to 15 minutes. After the lysis procedure had been performed twice, the two resulting inclusion body pellets were each suspended in 50 mL wash buffer (50 mM Tris, 100 mM NaCl, 100 mM EDTA, 0.5% (v/v) Triton X-100, pH 8.0), homogenized, sonicated for 30 seconds at an amplitude of 35%, and centrifuged at 27,000×g for 15 minutes. This wash procedure was repeated twice more. The resulting inclusion body pellets were then suspended in 50 mL of a second wash buffer (50 mM Tris, 10 mM EDTA, pH 8.0), homogenized, sonicated for 30 seconds at an amplitude of 35%, and centrifuged at 15,000×g for 15 minutes. This second wash step was performed twice. The washed inclusion body pellets were then unfolded by being homogenized into 10 mL of a 7.12 M guanidine hydrochloride solution. This solution of unfolded streptavidin in guanidine hydrochloride was stirred at room temperature for an hour, after which it was centrifuged at 12,000×g for 10 minutes. The supernatant was then added dropwise at a rate of 30 mL/h to 1 L of chilled PBS that was being stirred vigorously. This rapid dilution of the streptavidin and guanidine hydrochloride allowed for the streptavidin to fold properly. The folded streptavidin in PBS was stirred overnight at 4° C., and was then centrifuged at 7,000×g for 15 minutes to remove insoluble protein. The supernatant was filtered using a 0.45 µm bottle-top filter, and was then stirred while ammonium sulfate was slowly added to a concentration of 1.9 M. This concentration of ammonium sulfate serves to precipitate out impurities. The solution was stirred for at least 3 hours at 4° C., after which it was centrifuged at 7,000×g for 15 minutes to pellet the precipitated impurities. The supernatant was filtered using a 0.45 µm bottle-top filter, and was then stirred while ammonium sulfate was added to a total concentration of 3.68 M. This concentration of ammonium sulfate precipitates the streptavidin. The solution was stirred for at least 3 h at 4° C. before being centrifuged at 7,000×g for 20 minutes to pellet the streptavidin. The supernatant was discarded, and the pelleted streptavidin was suspended in 20 mL of Iminobiotin Affinity Chromatography (IBAC) binding buffer (50 mM Sodium Borate, 300 mM NaCl, pH 11.0). This streptavidin solution was then allowed to flow through 5 mL of Pierce Iminobiotin Agarose (Thermo Scientific) in a gravity flow column (G-Biosciences) that had been rinsed with DI water and pre-equilibrated with IBAC binding buffer. The column was next washed with 20 column volumes of IBAC binding buffer, and the streptavidin was eluted from the column with 6 column volumes of elution buffer (20 mM KH₂PO₄, pH 2.2). The eluate was collected, dialyzed into PBS, and concentrated using a 10 kDa MWCO centrifugal filter (Millipore Sigma). The concentration of streptavidin was quantified by measuring the UV absorption at 280 nm.

### Expression and Purification of 0304-3H3 Antibody

The genes encoding the variable regions of the heavy chain and light chain of the 0304-3H3 antibody (Chi et al, 2020) were cloned into the TGEX-HC and TGEX-LC vectors (Antibody Design Labs), respectively, by Gene Universal, Inc. (Newark, DE). The plasmids were co-transfected in a 2:1 light chain to heavy chain ratio into 1 Expi293F cells using the ExpiFectamine Transfection Kit (Thermo Fisher Scientific) and associated protocol. After a 4-day incubation, the culture was centrifuged at 5,500×g for 20 minutes. The supernatant was diluted in PBS and filtered before being purified by using a 1 mL MabSelect SuRe column (Cytiva) according to the manufacturer’s protocol. The concentration of the purified 0304-3H3 antibody was quantified using the BCA assay (Thermo Scientific).

### In Vitro Biotinylation of AviTagged Proteins

The BirA-500 kit (Avidity LLC) and general protocol were used to biotinylate the AviTagged MS2 and S2 proteins. In brief, the proteins were buffer exchanged into 20 mM Tris, 20 mM NaCl, pH 8.0. The concentration of protein in solution was adjusted to either 45 µM for MS2 or 15 µM for S2 and S2_{mutS2′} before adding the recommended amount of Biomix B (a proprietary mixture of biotin, ATP, and magnesium acetate). The recommended amount of BirA was added to the MS2 solution, while three times the recommended amount of BirA was added to the S2 solutions. These solutions were incubated at 37° C. for 2 h while shaking vigorously. After the two-hour incubation, the solutions were moved to a nutator at 4° C. for overnight incubation. Finally, the biotinylated proteins were separated from the biotinylation reagents using a Superdex 200 increase 10/300 column (Cytiva) and quantified by using the BCA assay (Thermo Scientific).

### Assembly of MS2-SA VLP

The assembly of MS2-SA VLP has been previously described (Chiba et al., 2020). Approximately 1 mL of biotinylated MS2 at a concentration of about 0.7 mg/mL was added 2.5 µL at a time to stirred streptavidin that was in approximately 20-times molar excess and at a concentration of around 60 mg/mL. This mixture was stirred for 30 minutes at room temperature before the MS2-SA VLP was separated from excess streptavidin using a Superdex 200 increase 10/300 column (Cytiva). To quantify the purified MS2-SA VLP, a small sample of the MS2-SA VLP in Nu-PAGE lithium dodecyl sulfate (LDS) sample buffer (Invitrogen) was heated at 90° C. for at least 10 minutes and run on an SDS-PAGE gel with heated streptavidin standards of known mass.

### Assembly of VLP-S2 and VLP-S2_{mutS2′}

MS2-SA and biotinylated S2 or S2_{mutS2′} were mixed in a ratio determined using analytical SEC. For example, molar ratios of 5:1, 4:1, 3:1, 2:1, 1:1, 1:2, 1:3. 1:4 or 1:5 may be employed Mixtures consisting of 5 µg of S2 or S2_{mutS2′} and varying amounts of MS2-SA were run through a Superdex 200 increase 10/300 SEC column (Cytiva). The ratio of the mixture with the least amount of MS2-SA that resulted in a chromatogram without a peak corresponding to excess S2 or S2_{mutS2′} was the stoichiometric ratio used to generate VLP-S2 and VLP-S2mutS2′ for characterization and immunization.

### Sds-page

Protein samples were diluted with 5 µL of Nu-PAGE lithium dodecyl sulfate (LDS) sample buffer (Invitrogen). These protein samples and PageRuler Plus Prestained Protein Ladder (Thermo Scientific) were loaded into the wells of a 4-12% Bis-Tris gel (Invitrogen). The gel was run in MES-SDS buffer at 110 V for 60 minutes while being chilled at 4° C. The gel was stained with SimplyBlue SafeStain (Invitrogen), destained, and imaged using the ChemiDoc MP imaging system (Bio-Rad).

### Characterization of S2, S2_{mutS2′}, VLP-S2, and VLP-S2mutS2′ by ELISA

Antigen (0.1 µg S2 and S2mutS2′ – alone and on VLP) was coated onto Nunc MaxiSorp 96-well flat-bottom plates (Invitrogen). The antigen solution was incubated for 1 h, before the wells were emptied and 5% BSA (Millipore) in PBST (PBS with 0.05% Tween-20) was added to the wells. This BSA solution remained in the wells for 45 minutes, after which it was discarded from the plate and each well was washed with 200 µL of PBST three times. Next, primary antibody (0304-3H3) was diluted in 1% BSA in PBST and a final volume of 100 µL was added to each well. The moles of antibody per well were equivalent to the moles of S2 trimer that had been coated in the 19 well. The plate was left to incubate with the primary antibody for an hour, after which the plate was emptied, and each well was washed with 200 µL of PBST three times. Then 100 µL of the secondary antibody, horseradish peroxidase-conjugated anti-human IgG Fc fragment antibody (MP Biomedicals; 1:5,000 dilution) in 1 % BSA in PBST was added to each well. The secondary antibody solution remained in the plate for 1 h, after which the solution was discarded, and the wells of the plate were washed with 200 µL of PBST three times. The plate was then developed by adding 100 µL of TMB substrate solution (Millipore) to each well. The reaction was stopped after three minutes by adding 0.16 M sulfuric acid to each well. The absorbance of each well was then read at 450 nm using a Spectramax i3x plate reader (Molecular Devices).

### Dls

MS2-SA VLP was diluted in PBS to 100 µL such that there was 1 µg of SA in solution. VLP-S2 and VLP3 S2_{mutS2′} were each diluted in PBS to 100 µL such that there was 5 µg of S2 in solution. Each 100 µL solution was then pipetted into a UVette (Eppendorf), which was inserted into a DynaPro NanoStar Dynamic Light Scattering detector (Wyatt Technology). Dynamics software (Wyatt Technology) brought the temperature of the measurement cell to 25° C. The detector then proceeded with the measurement. Each measurement was the result of 10 acquisitions and was output as % Intensity, which could be converted to % Mass using the Isotropic Spheres model.

### Negative Stain Transmission Electron Microscopy

Conventional native-stain transmission electron microscopy (TEM) was performed, as described previously (Booth et al., 2018). Briefly, 4 µl of diluted samples were applied onto glow-discharged mesh copper grids (CF300-Cu; Electron Microscopy Science, PA), washed with PBS (1X), stained in droplets of 1 % phosphotungstic acid (PTA, PH 6~7) for 1 min. The grids were then blotted from the grid backside and air-dried inside a petri dish for at least 30 min under room temperature to minimize the negative-stain artifacts of flattening and stacking (Jung & Mun, 2018). The negative-stain grids were imaged in low-dose mode (50 e-/Å), using a Talos L120C transmission electron microscope (Thermo Fisher Scientific, previously FEI, Hillsboro, OR) at 120 kV, images were acquired on a 4k × 4k Ceta CMOS camera microscope (Thermo Fisher Scientific), using SerialEM 3.840.

### Plunge Freezing and Cryo-electron Microscopy

4 µl of the VLP suspension was added to a glow discharged copper grid (C-Flat 1.2/1.3, 400 mesh, Protochips) with an extra layer of carbon (~2 nm) on the holey carbon surface. Grids were plunge frozen using a Vitrobot Mark IV (ThermoScientific) and stored in liquid nitrogen until imaging. Cryo-electron microscopy (cryo-EM) imaging was performed on a Titan Krios (ThermoScientific Hillsboro, OR, USA) operated at 300 kV. Images (defocus of -2~5 µm) were recorded on a post-GIF Gatan K3 camera in EFTEM mode (2.176 Å/pixel) with a 20-eV slit, CDS counting mode, using SerialEM 3.8 (Matronarede, 2005). A total dose of 30~40 e/ Å2 was used and 40 frames were saved (~1.2 e/ Å2 per frame). Frames were motion-corrected in MotionCor241. Images were low pass filtered to 10 Å2 for better visualization and contrast using EMAN2 (Galaz-Montoya et al., 2015).

### Cells and Virus

Vero E6/TMPRSS2 cells obtained from the National Institute of Infectious Diseases, Japan (Imai et al., 2020) were maintained in high glucose Dulbecco’s modified Eagle’s medium (DMEM) containing 10% fetal bovine serum (FBS) and antibiotic/antimycotic solution along with G418 (1 mg/ml). SARS-CoV-2/UT-NCGM02/Human/2020/Tokyo (NCGM02) was amplified on Vero E6 TMPRSS2 cells and used as the challenge virus in the vaccinated hamster study.

### Hamster Study

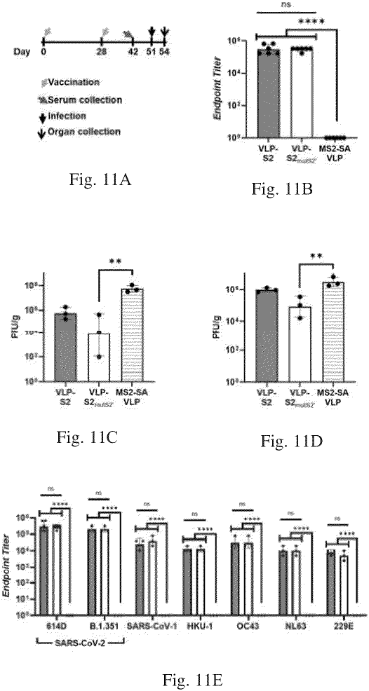

The schedule of the hamster study is depicted in FIG. 11a. Golden Syrian hamsters (females; 4-5 weeks old) were immunized with 20 µg of SARS-CoV-2 S2 protein presented on VLPs, a mutant S2 protein presented on VLPs, or VLP without the S protein, by subcutaneous inoculation. Addavax (InvivoGen) was added at an equal volume and thoroughly mixed with each vaccine preparation before inoculation. Animals were infected by intranasal inoculation with 10³ plaque-forming units of SARS-CoV-2 while under isoflurane anesthesia. Three days after infection, animals were humanely sacrificed and lung tissue and nasal turbinate samples were collected to measure amount of virus.

Virus titers in the tissues were determined on confluent Vero E6/TMPRSS2 cells by infecting cells with 100 µl of undiluted or 10-fold dilutions (10⁻¹ to 10⁻⁵) of clarified lung and nasal turbinate homogenates. After a 30-minute incubation, the inoculum was removed, the cells were washed once, and then overlaid with 1% methylcellulose solution in DMEM with 5% FBS. The plates were incubated for three days, and then the cells were fixed and stained with 20% methanol and crystal violet in order to count the plaques.

### Detection of Antibodies Against the SARS-CoV-2 S2 in Immunized Hamsters.

ELISAs were performed using recombinant spike SARS-CoV-2 proteins either produced in Expi293F cells (Thermo Fisher Scientific) and then C-terminal His-tag purified by using TALON metal affinity resin (Wuhan and B.1.351 spike antigens) or purchased from Sino Biological (229E, OC43, HKU-1, NL63,and CoV-1 strain Tor2 spike antigens). ELISA plates were coated overnight at 4° C. with 50 µl of spike antigen at a concentration of 2 µg/ml in PBS. After blocking with PBS containing 0.1% Tween 20 (PBS-T) and 3% milk powder, the plates with incubated in duplicate with heat-inactivated serum diluted in PBS-T with 1% milk powder. A hamster IgG secondary antibody conjugated with horseradish peroxidase (Invitrogen; 1:7,000 dilution) was used for detection. Plates were developed with SigmaFast o-phenylenediamine dihydrochloride solution (Sigma), and the reaction was stopped with the addition of 3 M hydrochloric acid. The absorbance was measured at a wavelength of 490 nm (OD490). Background absorbance measurements from serum collected before immunization was subtracted from serum collected before challenge for each dilution. IgG antibody endpoint titers were defined as the highest serum dilution with an OD490 cut-off value of 0.15.

### Neutralization Assay

Virus (NCGM02; ~100 PFU) was incubated with the same volume of two-fold dilutions of heat-inactivated serum for 30 minutes at 37° C. The antibody/virus mixture was added to confluent Vero E6/TMPRSS2 cells that were plated at 30,000 cells per well the day prior in 96-well plates. The cells were incubated for 3 days at 37° C. and then fixed and stained with 20% methanol and crystal violet solution. Virus neutralization titers were determined as the reciprocal of the highest serum dilution that completely prevented cytopathic effects.

### Statistics and Reproducibility

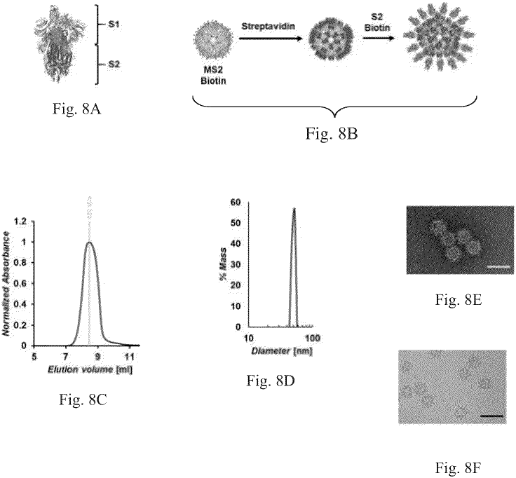

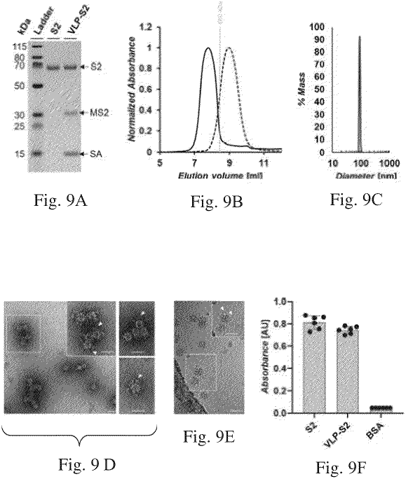

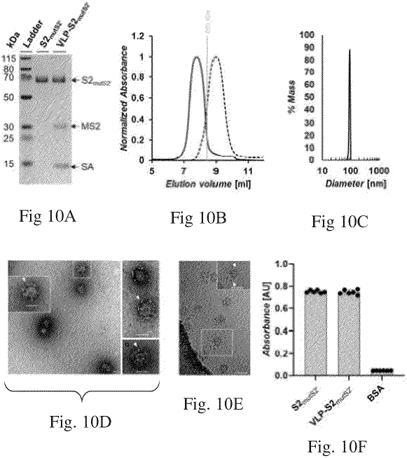

In vitro charactenzations of the binding of 0304-3H3 to the VLP-S2 and VLP-S2 using ELISA (FIGS. 9e and 10e) were each conducted twice independently with three technical replicates for each condition. The data are presented as the mean + SD. For in vivo characterization, there were three groups (receiving either VLP-S2, VLP-S2_{mutS2′}, or MS2-SA VLP) each with three hamsters (n=3). To determine the resulting endpoint titers against the SARS-CoV-2 spike protein (FIG. 11b; Table 3), two independent assays were conducted using sera from each hamster. Significance was determined by a one-way analysis of variance (ANOVA) and Tukey post-hoc multiple comparison between groups (α = 0.05). All other endpoint titers and neutralizing titers (FIG. 11e; Table 4) were determined by conducting an assay using sera from each of the three hamsters per group. The data are presented as the geometric mean with the geometric SD factor and significance was determined by a one-way analysis of variance (ANOVA) and Tukey post-hoc multiple comparison between groups (α = 0.05). Viral titers in the lungs and nasal turbinates of hamsters immunized with either VLP-S2, VLP-S2mutS2′, or MS2-SA VLP 3 days after SARS-CoV-2 infection (FIGS. 8c, d) were presented as the geometric mean with geometric SD (n=3) and the significance was determined by a one-way analysis of variance (ANOVA) and Dunnett post-hoc multiple comparison between groups (α = 0.05). For all tests of significance, assumptions of the normality of residuals and homogeneity of variance were validated by the D′Agostino-Pearson test and the Brown-Forsythe test, respectively. All statistical analysis was carried out using Prism (GraphPad).

TABLE 3

Antibody responses to VLP-S2 and VLP-S2_{mutS2′} after prime and boost in Syrian hamsters
Spike IgG Endpoint Titer^{a}

| | SARS-CoV-2 | | | | SARS-CoV-1 | | HKU-1 | | OC43 | | NL63 | | 229E | |
|---|---|---|---|---|---|---|---|---|---|---|---|---|---|---|
| | 614D | | B.1.351 | | | | | | | | | | | |
| Vaccine Group | Geo metric Mean | Geo metric SD Factor | Geo metric Mean | Geo metric SD Factor | Geo metric Mean | Geo metric SD Factor | Geo metric Mean | Geo metric SD Factor | Geo metric Mean | Geo metric SD Factor | Geo metric Mean | Geo metric SD Factor | Geo metric Mean | Geo metric SD Factor |
| MS2-SA VLP | <20 | – | <20 | – | <20 | – | <20 | – | <20 | – | <20 | – | <20 | – |
| VLP-S2 | 292,667 | 1.98 | 206,425 | 1.49 | 25,803 | 2.23 | 12,902 | 1.49 | 32,404 | 2.23 | 10,240 | 2.00 | 8,127 | 1.49 |
| VLP-S2_{mutS2′} | 291,930 | 1.33 | 206,425 | 1.49 | 40,960 | 2.00 | 12,902 | 1.49 | 32,510 | 2.23 | 10,240 | 2.00 | 5,120 | 2.00 |

TABLE 4

Neutralizing Antibody Titer^{b}

| | SARS-CoV-2 | B.1.351 | B.1.617.2 |
|---|---|---|---|
| Vaccine Group | Geometric Mean | Geometric Mean | Geometric Mean |
| MS2-SA VLP | <20 | <20 | <20 |
| VLP-S2 | <20 | <20 | <20 |
| VLP-S2_{mutS2′} | <20 | <20 | <20 |

^{a} Viral antibody endpoint titers against the SARS-CoV-2 spike (three animals in each group). Endpoint titers using 2-fold diluted sera were expressed as the reciprocal of the highest dilution with an optical density at 490 nm cutoff value >0.15; sera were collected on day 42 after the initial immunization.
^{b} Viral neutralization titers (three animals in each group). Endpoint titers using 2-fold diluted sera were expressed as the reciprocal of the highest dilution that completely prevented cytopathic effects; sera were collected on day 42 after immunization.

### Results

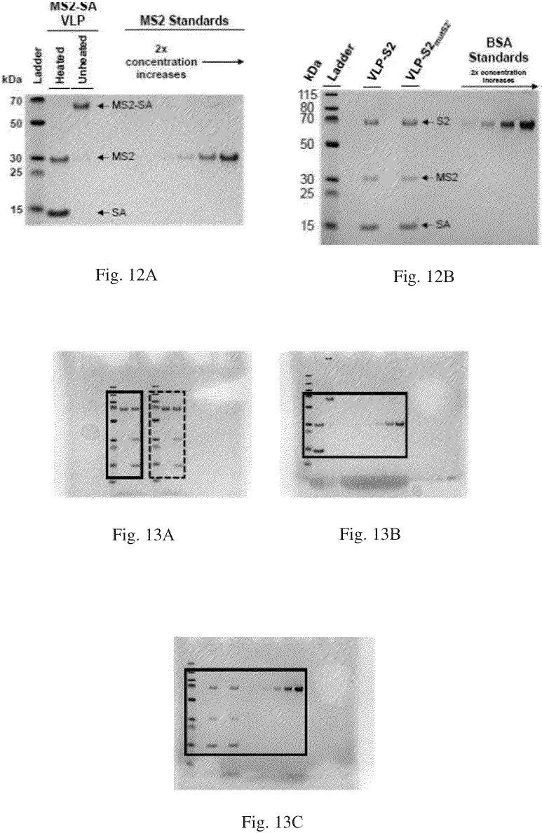

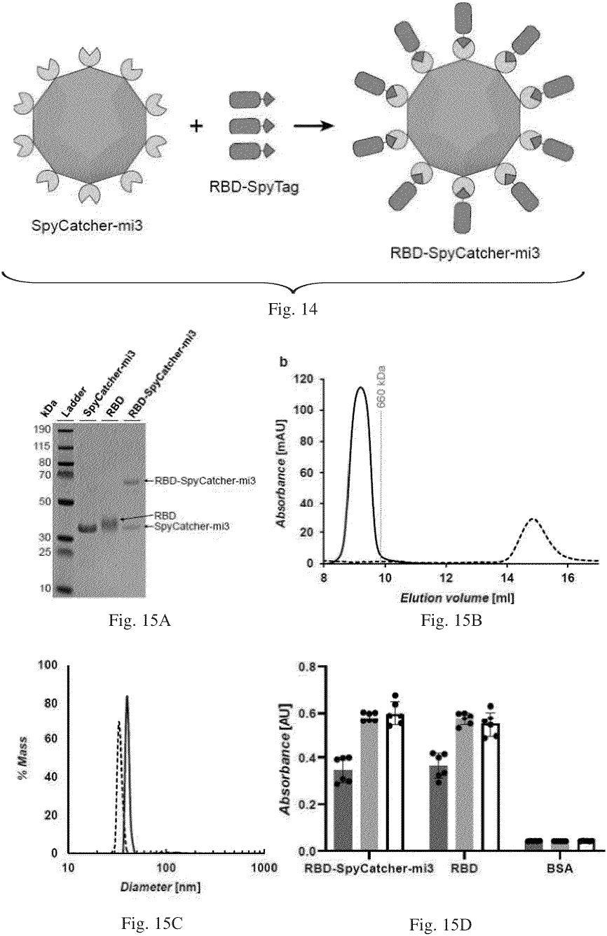

Streptavidin-coated VLPs were used to display biotinylated protein antigens such as the SARS-CoV-2 spike protein (Example 1) and DIII of the Zika virus envelope protein (Chiba et al., 2021; Castro et al., 2021). In this study VLPs were used to display the S2 subunit of the spike protein (FIG. 8b). The VLPs are based on the bacteriophage MS2 coat protein (Frietze et al., 2016); MS2 coat protein homodimers self-assemble into an icosahedral structure (Valegard et al., 1994). We used BL21(DE3) Escherichia coli (E. Coli) to express a single chain dimer of the MS2 coat protein with an AviTag inserted in a surface loop that had been shown to tolerate peptide insertions (see MS2-AviTag) (Peabody et al., 2008). The inserted AviTag allowed for site-specific biotinylation of each coat protein dimer. After expression, the VLPs were purified using Capto Core 700 resin and size exclusion chromatography (SEC). The VLPs were then biotinylated and subsequently separated from the biotinylation reagents using SEC. The biotinylated MS2 VLPs were added dropwise to a large excess of stirred streptavidin (SA), which had been expressed as inclusion bodies, refolded, and purified using iminobiotin affinity chromatography (Chiba et al., 2021; Castro et al., 2021). The resulting MS2-SA VLPs were separated from excess streptavidin using size exclusion chromatography. Consistent with prior characterization (Chiba et al., 2021), SDS-PAGE analysis of the MS2-SA VLPs indicated that there were approximately 70 streptavidin molecules bound to each MS2 biotin VLP (FIG. 12a). In addition, the MS2-SA VLPs were found to be pure and homogenous in size based on characterization by SEC (FIG. 1c), dynamic light scattering (DLS; FIG. 1d), negative-stain transmission electron microscopy (NS-TEM; FIG. 8e) and cryo-electron microscopy (cryo-EM; FIG. 8f).

### MS2-AviTag

MASNFTQFVLVDNGGTGDVTVAPSNFANGVAEWISSNSRSQAYKVTCSVR
QSSAQNRKYTIKVEVPKVATQTVGGVELPVAAWRSYLNMELTIPIFATNS
DCELIVKAMQGLLKDGNPIPSAIAANSGIYASNFTQFVLVDNGGGLNDIF
EAQKIEWHETGDVTVAPSNFANGVAEWISSNSRSQAYKVTCSVRQSSAQN
RKYTIKVEVPKVATQTVGGVELPVAAVVRSYLNMELTIPIFATNSDCELl
VKAMQGLLKDGNPIPSAIAANSGIY

(SEQ ID NO:4), or a polypeptide with at least 80%, 85%, 90%, 92%, 94%, 95%, 97%, 98% or 99% amino acid sequence identity thereto.

### S2

METDTLLLWVLLLWVPGSTGDSVASQSIIAYTMSLGAENSVAYSNNSIAI
PTNFTISVTTEILPVSMTKTSVDCTMYICGDSTECSNLLLQYGSFCTQLN
RALTGIAVEQDKNTQEVFAQVKQIYKTPPIKDFGGFNFSQILPDPSKPSK
RSPIEDLLFNKTTLADAGFlKQYGDCLGDIAARDLICAQKFNGLTVLPPL
LTDEMIAQYTSALLAGTITSGWTFGAGPALQIPFPMQMAYRFNGIGVTQN
VLYENQKLIANQFNSAIGKIQDSLSSTPSALGKLQDVVNQNAQALNTLVK
QLSSNFGAISSVLNDILSRLDPPEAEVQIDRLITGRLQSLQTYVTQQLIR
AAEIRASANLAATKMSECVLGQSKRVDFCGKGYHLMSFPQSAPHGWFLHV
TYVPAQEKNFTTAPAICHDGKAHFPREGVFVSNGTHWFVTQRNFYEPQII
TTDNTFVSGNCDVVIGIVNNTVYDPLQPELDSFKEELDKYFKNHTSPDVD
LGDISGINASVVNIQKEIDRLNEVAKNLNESLIDLQELGKYEQGSGYIPE
APRDGQAYVRKDGEWVLLSTFLGGLNDIFEAQKIEWHEHHHHHH

(SEQ ID NO:5), or a polypeptide with at least 80%, 85%, 90%, 92%, 94%, 95%, 97%, 98% or 99% amino acid sequence identity thereto.

### S2mutS2′

METDTLLLWVLLLWVPGSTGDSVASQSIIAYTMSLGAENSVAYSNNSIAI
PTNFTISVTTEILPVSMTKTSVDCTMYICGDSTECSNLLLQYGSFCTQLN
RALTGIAVEQDKNTQEVFAQVKQIYKTPPIKDFGGFNFSQILPDPSKPSG
GSPIEDLLFNKVTLADAGFIKQYGDCLGDIAARDLICAQKFNGLTVLPPL
LTDEMIAQYTSALLAGTITSGWTFGAGPALQIPFPMQMAYRFNGIGVTQN
VLYENQKLIANQFNSAIGKIQDSLSSTPSALGKLQDVVNQNAQALNTLVK
QLSSNFGAISSVLNDILSRLDPPEAEVQIDRLITGRLQSLQTYVTQQLIR
AAEIRASANLAATKMSECVLGQSKRVDFCGKGYHLMSFPQSAPHGVVFLH
VTYVPAQEKNFTTAPAICHDGKAHFPREGVFVSNGTHWFVTQRNFYEPQI
ITTDNTFVSGNCDVVIGIVNNTVYDPLQPELDSFKEELDKYFKNHTSPDV
DLGDISGINASWNIQKEIDRLNEVAKNLNESLIDLQELGKYEQGSGYIPE
APRDGQAYVRKDGEVWLLSTFLGGLNDIFEAQKIEWHEHHHHHH

(SEQ ID NO:6), or a polypeptide with at least 80%, 85%, 90%, 92%, 94%, 95%, 97%, 98% or 99% amino acid sequence identity thereto.

Biotinylated S2 was next produced such that it could be displayed on the MS2-SA VLPs. Expi293F mammalian cells were used to express the HexaPro31 variant of the SARS-CoV-2 spike protein’s S2 subunit with an N-terminal signal peptide, a C-terminal trimerization domain to promote stability, a C-terminal AviTag for biotinylation, and a C-terminal his-tag for purification. The expressed S2 was purified using immobilized metal affinity chromatography (IMAC) and was then biotinylated in vitro. The biotinylated S2 was separated from biotinylation reagents using size exclusion chromatography 1 and could then be displayed on the MS2-SA VLPs.

To determine the appropriate ratio of S2 to add to MS2-SA VLPs, analytical SEC was used. Mixtures of the two proteins were made that contained a constant amount of S2 and varying amounts of MS2-SA VLPs. The ratio of the mixture with the least amount of MS2-SA VLPs that displayed no indication of excess S2 in an SEC chromatogram was determined to be the approximate stoichiometric ratio. Further analysis by SDS-PAGE indicated that this stoichiometric ratio resulted in approximately 30 S2 molecules conjugated to each MS2-SA VLP (FIG. 12b). The MS2-SA and biotinylated S2 were mixed in this ratio to create the VLP-S2 immunogen.

The VLP-S2 immunogen was characterized in vitro using several different bioanalytical techniques. First, the proteins that made up VLP-S2 were characterized by SDS polyacrylamide gel electrophoresis (SDS-PAGE), which indicated that the proteins were pure (FIG. 9a). In addition, comparison of the molecular weight ladder to the bands representing deglycosylated S2 (~63 kDa), biotinylated MS2 (~29 kDa), and monomeric streptavidin (~15 kDa) demonstrated that these proteins aligned as expected with molecular weight standards. The VLP-S2 was also analyzed using analytical SEC, where chromatograms were generated for VLP-S2, S2 alone, and the molecular weight standard thyroglobulin (FIG. 9b). The resulting UV trace corresponding to the VLP-S2 contained a single peak that appeared before the peak for S2 alone. Therefore, the VLP-S2 was free of excess S2 and was generally uniform in size. To obtain a direct size measurement of the VLP-S2, Dynamic Light Scattering (DLS) (FIG. 9c), NS-TEM (FIG. 9d), and cryo-EM (FIG. 9e) were used. The DLS measurements indicated that the VLP-S2 construct was approximately 90 nm in diameter. Characterization of the VLP-S2 by NS-TEM and cryo-EM confirmed the presence and coating of the S2 protein on the surface of the MS2-SA VLP. NS-TEM analysis suggested that VLP-S2 was ~65 nm in diameter on average (n=300). The larger size indicated by DLS may be a result of the fact that scattering intensity is proportional to the sixth power of the radius, giving rise to a disproportionately higher weighting of larger particles. We next used ELISA to probe the binding of the anti-S2 monoclonal antibody 0304-3H3 (Chi et al., 2020) to S2 and VLP-S2 (FIG. 9f). This antibody bound to both the S2 and VLP-S2, suggesting that S2 retained its bioreactivity after conjugation to VLPs.

In addition to the VLP-S2, VLP-S2_{mutS2′} particles were generated. The VLP-S2_{mutS2′} 1 displayed an S2 variant (S2_{mutS2′}) that contained S2′ cut site residues that had been mutated to glycine residues. The purpose of this mutation was to prevent potential proteolytic cleavage of the S2 immunogen at the S2′ cut site. The VLP-S2_{mutS2′} was generated and characterized using the same procedures described above for the VLP-S2 (FIG. 10).

The in vivo efficacy of the VLP-S2 and VLP-S2_{mutS2′} was next assessed (FIG. 11a). Hamsters were immunized with either VLP-S2, VLP-S2_{mutS2′}, or MS2-SA VLP alone and were boosted 28 days later. Hamsters immunized with the VLP-S2 and VLP-S2_{mutS2′} generated high antibody titers against the S ectodomain (FIG. 11b; Table 2). To gauge whether immunization with VLP-S2 and VLP-S2_{mutS2′} was protective, the vaccinated hamsters were intranasally inoculated with 103 plaque-forming units of SARS-CoV-2/UT11 NCGM02/Human/2020/Tokyo (an early isolate that contains 614D) (Imai et al., 2020) 51 days after the initial immunization. The hamsters were then sacrificed 3 days after infection and viral titers in their lungs and nasal turbinates were quantified. The geometric mean viral titer in the lungs of hamsters immunized with VLP-S2 was nearly 100-fold lower than that of the hamsters immunized with MS2-SA VLP (FIG. 11c). The geometric mean viral titer in the lungs of hamsters immunized with VLP-S2_{mutS2′} was more than 7,000-fold lower than that of hamsters immunized with MS2-SA VLP – a statistically significant difference (FIG. 11c). These results demonstrate that immunization with the S2-based immunogens VLP-S2 and VLP-S2_{mutS2′} provides protection against SARS-CoV-2. Characterization of viral titers in the nasal turbinates of the immunized hamsters also indicated that the multivalent S2 constructs provided protection against SARS-CoV-2 (FIG. 11D). The geometric mean viral titers in the nasal turbinates of hamsters immunized with VLP-S2 and VLP-S2_{mutS2′} were respectively 3- and 36-fold lower than that of hamsters immunized with MS2-SA VLP.

Next, the breadth of the immune response generated by VLP-S2 and VLP-S2_{mutS2′} was evaluated using ELISA. Immunization with the multivalent S2 constructs elicited high antibody titers against the spike protein of not only the original Wuhan-Hu-1 SARS-CoV-2 (614D) (Wu et al., 2020), but also against the spike proteins of the SARS-CoV-2 variant B.1.351, SARS-CoV-1, and the four endemic human coronaviruses HKU-1, OC43, NL63, and 229E (FIG. 11e and Table 2). This substantial cross-reactivity suggests that immunization with multivalent S2-based immunogens may be a promising strategy for eliciting a broadly protective 1 response against coronaviruses.

Interestingly, despite this protection against a viral challenge, high antibody titers (FIG. 4b), and broad cross-reactivity (FIG. 11e and Table 4), sera from hamsters immunized with VLP-S2 and VLP-S2_{mutS2′} did not show neutralization activity in vitro against SARS-CoV-2 (614D) or the variants B.1.351 and B.1.617.2 (Table 4). This result suggests that the protection afforded to the hamsters through immunization with the multivalent S2 constructs might arise from other mechanisms, such as Fc effector functions. Fc effector functions have previously been identified as a mechanism by which S2-specific antibodies provide protection (Shiakolas et al., 2021). In addition, antibodies targeting the S2-analogous region of the influenza protein hemagglutinin (the stalk domain) are known to provide protection through Fc effector functions (DiLillo et al., 2014). While further studies will elucidate the exact mechanism of protection imparted by VLP-S2 and VLP-S2m_{utS2′}, the present results demonstrate that the multivalent S2 constructs are capable of eliciting a broadly cross-reactive immune response that protects against SARS-CoV-2. Therefore, the S2 subunit may be employed next-generation coronavirus vaccines designed to protect against emerging SARS-CoV-2 variants and other zoonotic coronaviruses with pandemic potential.

### Example 4

The persistence of the global SARS-CoV-2 pandemic has brought to the forefront the need for safe and effective vaccination strategies. In particular, the emergence of several variants with greater infectivity and resistance to current vaccines has motivated the development of a vaccine that elicits a broadly neutralizing immune response against all variants. In this study, a nanoparticle-based vaccine platform was used for the multivalent display of the receptor binding domain (RBD) of the SARS-CoV-2 spike (S) protein, the primary target of neutralizing antibodies. Multiple copies of RBD were conjugated to the SpyCatcher-mi3 protein nanoparticle to produce a highly immunogenic nanoparticle-based vaccine. RBD-SpyCatcher-mi3 vaccines elicited broadly cross-reactive antibodies that recognized the spike proteins of not just an early isolate of SARS-CoV-2, but also three SARS-CoV-2 variants of concern as well as SARS-CoV-1. Moreover, immunization elicited high neutralizing antibody titers against an early isolate of SARS-CoV-2 as well as four variants of concern, including the delta variant. Thus, RBD-SpyCatcher-mi3 may be employed as a broadly protective vaccination strategy.

### Methods

### Cloning of RBD Constructs, S Trimer, anti-RBD Antibodies, and SpyCatcher-mi3

Construct 2019-nCoV RBD-SpyTag, encoding amino acids 319-541 from the SARS-CoV-2 S protein sequence (UniProt PODTC2) followed by a GGSGG spacer, a SpyTag, and a 6×His-Tag, was enhanced for expression in mammalian cells and synthesized by Gene Universal Inc. (Newark, DE).

Sequences encoding the light and heavy chains of the CR3022 antibody (Yuan et al., 2020) (retrieved from PDB 6W41) and the S309 antibody (Pinto et al., 2020) (retrieved from PDB 6WPS) were cloned into the TGEX-LC and TGEX-HC vectors, respectively. To create the DNA construct for ACE2-Fc, residues 1-615 of ACE2 were cloned into the TGEX-HC vector. Codon optimization and DNA synthesis for all three constructs were carried out by Gene Universal Inc.

DNA encoding the SpyCatcher-mi3 fusion protein (Bruun et al., 2018) was cloned into pET-21a and synthesized by Gene Universal Inc. with no additional modifications. The DNA encoding the RBD constructs, ACE2-Fc, CR3022, and S309 was transformed into 5-α competent cells according to the manufacturer’s recommendations. Transformed cells were grown at 37° C. in 100 mL of 2×YT medium containing ampicillin. On the following day, the DNA was extracted and purified with an E.Z.N.A Plasmid Maxi Kit (Omega). The DNA coding for the mi3-SpyCatcher was transformed into BL21 (DE3) cells (New England Biolabs), which were grown overnight at 37° C. and frozen as glycerol stocks.

### Expression and Purification of RBD, S, ACE2-Fc, CR3022, and S309

RBD, ACE2-Fc, CR3022, and S309 constructs were expressed in HEK293F suspension cells using the ExpiFectamine™ 293 transfection kit (A14524, Gibco) according to the manufacturer’s protocol. Cells expressing RBD were harvested 4 days after transfection, centrifuged, and the supernatants were thoroughly dialyzed against IMAC Binding Buffer (100 mM Tris, 150 mM NaCl, 20 mM imidazole, pH 8.0).

Dialyzed proteins were poured over Ni-NTA resin that had been pre-equilibrated with 10 column volumes (CVs) of IMAC Binding Buffer. The resin was then washed with at least 20 CVs of IMAC Binding Buffer and eluted with 5 CVs of IMAC Elution Buffer (100 mM Tris, 150 mM NaCl, 400 mM imidazole, pH 8.0). The eluates were collected and concentrated using an Amicon spin filter (EMD MilliPore) with a 10 kDa MWCO. The concentrated RBD protein was further purified with a Superdex Increase 200 10/300 GL column for the removal of high molecular weight impurities. Cells transfected with either ACE2-Fc, CR3022, or S309 DNA were harvested 5 days after transfection and centrifuged to remove the cells and cell debris. The resulting supernatant was diluted in PBS and loaded onto a MabSelect SuRe column (GE) to be purified according to the manufacturer’s recommendations. The eluate containing the desired protein was then stored at 4° C.

### Expression and Purification of SpyCatcher-mi3

Cells transformed with the DNA coding for SpyCatcher-mi3 were used for a 5 mL starter culture which was further scaled up (after growing for 12-16 hrs) to 1 L of 2xYT media containing kanamycin. Cells were grown at 37° C. until the OD600 reached 0.8. The temperature was reduced to 22° C. and isopropyl β-D-1-thiogalactopyranoside (IPTG) was added to a final concentration of 0.5 mM. Cells were allowed to grow overnight before harvest and were then centrifuged at 7,000 xg for 7 minutes.

Cell lysis and protein purification were performed according to the protocol described by Bruun et al. (2018). In brief, the cell pellet was resuspended in 20 mL of CaptureSelect Equilibration Buffer (25 mM Tris, 150 mM NaCl, pH 8.5) containing 2 µg of lysozyme, 125 units of benzonase, and half of a tablet of SigmaFast EDTA-free (Sigma Aldrich) protease inhibitor cocktail. The mixture was incubated at room temperature for 1 hr and then sonicated for 5 minutes with 5 s on, 5 s off pulses. Following sonication, the solution was centrifuged for 30 minutes at 17,000 xg, and the supernatant was poured over 5 mL of pre-equilibrated CaptureSelect C-tag Affinity Matrix (ThermoFisher Scientific). The resin was washed with 10 CVs of CaptureSelect Equilibration Buffer and eluted with CaptureSelect Elution Buffer (20 mM Tris, 2 M MgCl2, pH 8.5). All purification steps were performed at 4° C. The eluate containing the protein of interest was dialyzed against 25 mM Tris, 150 mM NaCl, pH 8.5, overnight with a 50 kDa MWCO SpectraPor dialysis membrane (Repligen) and concentrated by spin filtration using a ViVaspin filter (Sartorius) with a 50 kDa MWCO. The concentrated protein was further purified by SEC using a Superdex Increase 200 10/300 GL column. Fractions corresponding to chromatogram peaks were analyzed by DLS, while fractions containing large amounts of aggregates were discarded. The remaining fractions were concentrated and stored at 4° C.

### Conjugation of RBDs to SpyCatcher-mi3 Nanocages

Small scale reactions between RBD constructs and SpyCatcher-mi3 were initially set up to determine desirable stoichiometric ratios. For example, molar ratios of 5:1, 4:1. 3:1, 2.5:1, 2:1, 1.5:1, 1:1, 1: 1.5, 1:2, 1:2.5, 1:3, 1:4 or 1:5 may be employed Mixtures were allowed to react overnight and the extent of RBD conjugation to the scaffold was determined by SDS-PAGE. Reactions that showed a consumption of greater than 90% of RBD did not undergo any additional purification steps. Reactions that had lower yields were further purified by size exclusion chromatography (SEC) using a GE Superdex 200 Increase 10/300 GL and eluate fractions containing the reaction product were concentrated. Sample purity and final RBD concentration were determined by SDS-PAGE.

### Sds-page

Protein samples were diluted in Nu-PAGE lithium dodecyl sulfate (LDS) loading buffer (Invitrogen) to a final quantity of 1 µg. 15 µL of protein samples and 2 µL of PageRuler Plus Prestained Protein Ladder were added to the wells of a 4-12% Bis-Tris gel (Invitrogen). Gels were run in MES-SDS buffer at 120 V for 50 minutes, then stained with Imperial Protein Stain (ThermoFisher Scientific). After destaining, gels were imaged using the ChemiDoc MP imaging system and Image Lab 5.2.1 software (Bio-Rad).

### Dynamic Light Scattering

100 µL samples of SpyCatcher-mi3 or RBD-SpyCatcher-mi3 at a concentration of ~0.5 µg/µL were added to a UVette (Eppendorf). Dynamics software and a DynaPro NanoStar Dynamic Light Scattering detector were used to collect five acquisitions for each measurement. Acquisitions were averaged and results were displayed as % Mass using the Isotropic Sphere model.

### Analytical SEC

Samples of RBD and RBD-SpyCatcher-mi3, each containing 20 µg of RBD, were diluted in 1 mL of PBS. Samples were loaded into the sample loop which was then flushed with PBS to inject sample onto a Superdex 200 Increase 10/300 Column (Cytiva) using Unicorn 7 (Cytivia) control system. Protein was eluted with one column volume of PBS flowing at a flow rate of 0.5 mL/min. UV absorbance at 205, 210, and 280 nm was monitored.

### Elisa

96-well plates were coated with 50 µL of RBD at 4 µg/mL and incubated at room temperature for 1 hr. Wells were blocked with 100 µL of 5% (w/v) bovine serum albumin (BSA) diluted in PBS containing 0.1% tween-20 (PBST) for 1 hr. Blocking was followed by three washes with 100 µL of PBST and a 1 hr incubation with 50 µL of ACE2-Fc, S309, or CR3022, diluted to a final concentration of 1 µg/mL in PBST with 1% BSA. The plates were washed three more times and incubated with horseradish peroxidase (HRP)-conjugated anti-human antibodies diluted 20,000 fold in PBST with 1% BSA. After a 1 hr incubation, plates were washed three more times and developed by adding 50 µL of 3,3′,5,5′-tetramethylbenzidine (TMB) substrate. The reaction was quenched by adding 50 µL of Stop Solution (160 mM sulfuric acid) and the absorbance at 450 nm was measured.

### Immunizations

All immunizations were performed by ProSci Inc. (Poway, CA). Three mice were immunized with a solution either 14 µg of RBD conjugated to mi3-SpyCatcher or 16.8 µg of mi3-SpyCatcher mixed with an equal volume of Addavax adjuvant. On day 25, a boosting injection containing 20 µg of RBD antigen or 24 µg of mi3-SpyCatcher mixed with Addavax was administered. The mice were bled prior to the boost on day 25 and terminally bled on day 47. These immunizations and bleeds were carried out by ProSci Incorporated (Poway, CA) within their USDA licensed, registered and NIH/OLAW assured animal facility.

Other adjuvants which may be useful include but are not limited to aluminum, water in oil (W/O) emulsions, oil in water (O/W) emulsions, ISCOM, liposomes, nano- or micro-particles, muramyl di- and/or tripeptides, saponin, non-ionic block co-polymers, lipid A, cytokines, bacterial toxins, carbohydrates, and derivatized polysaccharides and a combination of two or more these adjuvants in an Adjuvant System

### (As).

Exemplary classes of adjuvants include but are not limited to agonists of TLR3, e.g., poly (I:C), agonists of TLR4, e.g., one or more components of bacterial lipopolysaccharide, e.g., monophosphoryl lipid A (MPLA), MPL®, and synthetic derivatives, e.g., E6020,agonists of TLR5, e.g., bacterial flagellin), agonists of TLR7, 8, e.g., single stranded RNA or imidazoquinolines (e.g., imiquimod, gardiquimod and R848),agonists of TLR9, e.g., CpG oligonucleotides and ISS immunostimulatory sequences, as well as imidazoquinolines, agonists of the NLRP3 inflammasome, e.g., chitosan, and dual TLR½ agonists, e.g., Pam3CSK4, a lipopeptide.

In one embodiment, the adjuvant comprises saponin, a natural product derived from tree bark, which may be combined with cholesterol or a cholesterol like molecule, e.g., squalene.

In one embodiment, the adjuvant comprises an oil-in-water (O/W) emulsion comprising, for example, MF59 or AS03 and optionally 2% squalene. In one embodiment, the adjuvant comprises two different adjuvants, e.g., MPL and a saponin such as QS21, for example, in liposome.

In one embodiment, the adjuvant comprises Freund’s Incomplete Adjuvant (IFA), MF59®, GLA-SE, IC31®, CAF01 AS03, AS04, or ISA51, and may include α-tocopherol, squalene and/or polysorbate 80 in an oil-in-water emulsion.

In one embodiment, the adjuvant comprises extracts and formulations prepared from Ayurvedic medicinal plants including but not limited to Withania somnifera, Emblica officinalis, Panax notoginseng, Tinospora cordifolia or Asparagus racemosus.

In one embodiment, the adjuvant comprises aluminum salts, saponin, muramyl di- and/or tripeptides, Bordetella pertussis, and/or cytokines.

In one embodiment, the adjuvant is not alum or an aluminum salt.

### Detection of anti-RBD Mouse Antibodies

ELISAs were performed using recombinant spike antigens produced from codon optimized cDNA expressed in Expi293F cells (Thermo Fisher Scientific). Recombinant proteins with a C-terminal HIS-tag were purified by using TALON metal affinity resin (Amanat et al., 2020). The recombinant spike antigen for SARS-CoV-1 strain Tor2 was purchased from Sin Biological. ELISA plates were coated overnight at 4° C. with 50 µl of spike antigen at a concentration of 2 µg/ml in phosphate-buffered saline (PBS). After blocking with PBS containing 0.1% Tween 20 (PBS-T) and 3% milk powder, the plates with incubated in duplicate with heat-inactivated serum diluted in PBS-T with 1% milk powder. A mouse IgG secondary antibody conjugated with horseradish peroxidase (Invitrogen; 1:5,000 dilution) was used for detection. Plates were developed with SigmaFast o-phenylenediamine dihydrochloride solution (Sigma), and the reaction was stopped with the addition of 3 M hydrochloric acid. The absorbance was measured at a wavelength of 490 nm (OD₄₉₀). Background absorbance measurements from pooled naive mouse serum was subtracted from serum collected after immunization for each dilution. IgG antibody endpoint titers were defined as the highest serum dilution with an OD₄₉₀ cut-off value of 0.15.

### Neutralization Assay

The following viruses were used in the neutralization assays: SARS-CoV-2/UT-NCGM02/Human/2020/Tokyo (S-614D), hCoV-19/Japan/TY7-501/2021 (P.1), nCoV-19/Japan/QHN001/2020 (B.1.1.7), hCoV-19/USA/MD-HP01542/2021 (B.1.351), and hCoV-19/USA/WI-UW-5250/2021 (B.1.617.2). The assays were performed on Vero E6 TMPRSS2 cells obtained from the National Institute of Infectious Diseases, Japan (Matsuyama et al., 2020). Viruses were incubated with the same volume of two-fold dilutions of heat-inactivated serum for 30 minutes at 37° C. The antibody/virus mixture was added to confluent Vero E6/TMPRSS2 cells that were plated at 30,000 cells per well the day prior in 96-well plates. The cells were incubated for 3 days at 37° C. and then fixed and stained with 20% methanol and crystal violet solution. Virus neutralization titers were determined as the reciprocal of the highest serum dilution that completely prevented cytopathic effects.

### SpyCatcher-mi3 Sequence

MKMEELFKKHKIVAVLRANSVEEAKKKALAVFLGGVHLIEITFTVPDADT
VIKELSFLKEMGAIIGAGTVTSVEQARKAVESGAEFIVSPHLDEEISQFA
KEKGVFYMPGVMTPTELVKAMKLGHTILKLFPGEVVGPQFVKAMKGPFPN
VKFVPTGGVNLDNVCEWFKAGVLAVGVGSALVKGTPVEVAEKAKAFVEKI
RGCTE

(SEQ ID NO:7), or a polypeptide with at least 80%, 85%, 90%, 92%, 94%, 95%, 97%, 98% or 99% amino acid sequence identity thereto.

### RBD Sequence

RVQPTESIVRFPNITNLCPFGEVFNATRFASVYAWNRKRISNCVADYSVL
YNSASFSTFKCYGVSPTKLNDLCFTNVYADSFVIRGDEVRQIAPGQTGKI
ADYNYKLPDDFTGCVIAWNSNNLDSKVGGNYNYLYRLFRKSNLKPFERDI
STEIYQAGSTPCNGVEGFNCYFPLQSYGFQPTNGVGYQPYRVVVLSFELL
HAPATVCGPKKSTNLVKNKCVNF

(SEQ ID NO:8), or a polypeptide with at least 80%, 85%, 90%, 92%, 94%, 95%, 97%, 98% or 99% amino acid sequence identity thereto.

### RBD-SpyTag Sequence

RVQPTESIVRFPNITNLCPFGEVFNATRFASVYAWNRKRISNCVADYSVL
YNSASFSTFKCYGVSPTKLNDLCFTNVYADSFVIRGDEVRQIAPGQTGKI
ADYNYKLPDDFTGCVIAWNSNNLDSKVGGNYNYLYRLFRKSNLKPFERDI
STElYQAGSTPCNGVEGFNCYFPLQSYGFQPTNGVGYQPYRVWLSFELLH
APATVCGPKKSTNLVKNKCVNFGGSGGSAHIVMVDAYKPTKHHHHHH

(SEQ ID NO:9), or a polypeptide with at least 80%, 85%, 90%, 92%, 94%, 95%, 97%, 98% or 99% amino acid sequence identity thereto.

### Results and Discussion

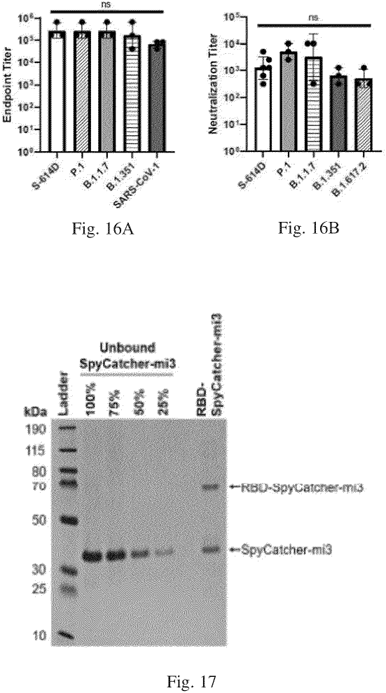

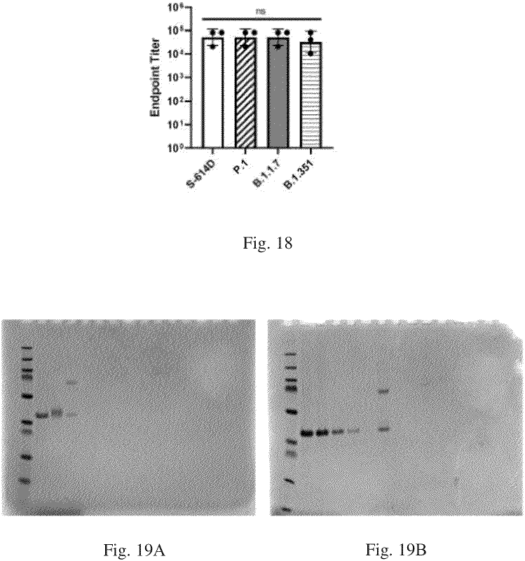

Protein nanoparticles that use SpyTag-SpyCatcher technology were used for the multivalent display of RBD (FIG. 14). The SpyCatcher-mi3 scaffold is based on a mutated aldolase protein from a thermophilic bacterium fused to the SpyCatcher protein that self-assembles into a dodecahedral 60-mer virus like particle (Bruun et al., 2018). The SpyCatcher allows for the conjugation of SpyTagged proteins through the formation of an isopeptide bond, making it a versatile and efficient platform. The SpyCatcher-mi3 was expressed in BL21 (DE3) competent E. coli cells. After cell lysis, Spycatcher-mi3 was purified with a CaptureSelect C-tag affinity column and size exclusion chromatography (SEC). Purity was assessed by SDS-PAGE (FIG. 15a).

Next, an RBD construct to be conjugated to SpyCatcher-mi3 was generated. The SpyTag sequence (AHIVMVDAYKPTK) was inserted at the C-terminus of the RBD (amino acids 319-541 of SARS-CoV-2 S protein) followed by a 6xHis-tag. The SpyTag enables conjugation to SpyCatcher-mi3, while the 6xHis-Tag enables purification by immobilized metal affinity chromatography (IMAC). The construct was expressed in Expi293F cells. Secreted protein was purified from the media using IMAC, then purified further with SEC to remove aggregates and other impurities.

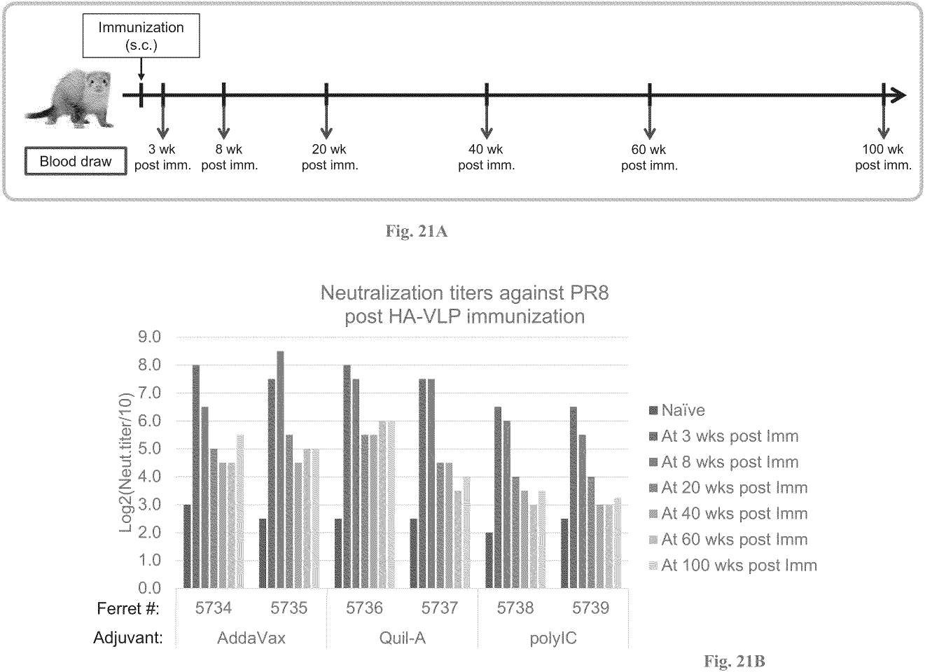

The RBD-SpyTag was mixed with SpyCatcher-mi3 overnight to generate RBD-SpyCatcher-mi3. Each particle contained approximately 30 RBD monomers, as determined by SDS-PAGE (FIG. 17). Characterization by SDS-PAGE showed two bands: the upper band at ~65 kDA corresponds to a SpyCatcher-mi3 monomer conjugated to a RBD monomer, and the lower band at ~34 kDA corresponds to a mi3 monomer alone (FIG. 15a). The RBD-SpyCatcher-mi3 was further characterized by SEC and dynamic light scattering (DLS), SEC chromatograms of RBD-SpyCatcher-mi3 and RBD (FIG. 15b) show the expected shift based on the large difference in molecular weight between the RBD monomer (25 kDa) and RBD-SpyCatcher-mi3 (~2.8 MDa). The curve for RBD-SpyCatcher-mi3 also only contains a single peak, which shows that there is very little unreacted RBD. DLS indicated a diameter of 40 nm for RBD-SpyCatcher-mi3 and a diameter of 34 nm for the SpyCatcher-mi3 nanoparticle alone (FIG. 15c). The increase in diameter for RBD-SpyCatcher-mi3 is consistent with the addition of an RBD layer to the outside of the nanoparticle and with the expected RBD diameter of 3 nm. To confirm that the RBD remained properly folded after conjugated to SpyCatcher-mi3, the binding of ACE2-Fc (an Fc fusion protein of the ACE2 receptor), and the RBD-specific antibodies S309 (a cross-neutralizing antibody)10 and CR3022 (a conformation-dependent antibody) (Ju et al., 2020) to RBD and RBD SpyCatcher-mi3 was characretrized by enzyme-linked immunosorbent assay (ELISA) (FIG. 15d). ELISA results confirmed the ability of both RBD and RBD-SpyCatcher-mi3 to be recognized by all three proteins, confirming the proper folding of important epitopes.

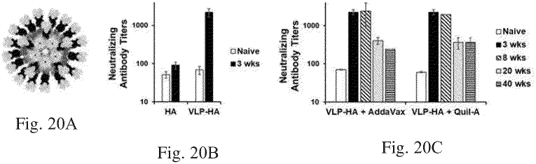

Next, mice were immunized with the RBD-SpyCatcher-mi3 vaccines to evaluate the immune response. RBD-SpyCatcher-mi3 mixed with AddaVax, a vaccine adjuvant consisting of an oil-in-water nano-emulsion, was administered to mice (n = 3), followed by a boost 25 days later. Mice were bled before the boost (25 days after the initial immunization) then terminally bled (47 days after the initial immunization) to collect sera and characterize the breadth of the antibody response. High titers against an early isolate of SARS-CoV-2 (S-614D) and 3 variants of concern – P.1, B.1.1.7, and B.1.351 - were seen after a single immunization with RBD-SpyCatcher-mi3 (FIG. 18). A second immunization boosted antibody titers against these SARS-CoV-2 variants and also elicited high antibody titers against SARS-CoV-1 (FIG. 16a and Table 3). Importantly, we also observed high neutralizing antibody titers against the early isolate of SARS-CoV-2 as well as 4 SARS-CoV-2 variants of concern – P.1, B.1.1.7, B.1.351, and B.1.617.2 (FIG. 14b and Table 3). RBD-SpyCatcher-mi3 demonstrated significantly higher neutralization titers against an early isolate of SARS-CoV-2 compared to those previously reported for an Addavax-adjuvanted monomeric RBD (Lederer et Al., 2020). There was no significant difference in the endpoint antibody titers and neutralizing antibody titers against the different strains (FIGS. 14a and 14b).

These titers were compared with some previously published reports for RBD-based vaccines. The assays used in the literature are not standardized and some reports have used different animal models. These differences in results must therefore be interpreted with caution. Tan et al. evaluated the neutralization potency of mice immunized with low doses of RBD-SpyVLPs using a live SARS-CoV-2 virus neutralization assay5. High neutralizing titers were seen, with the reciprocal of the dilution required for 50% reduction in the number of plaques ranging from 450-2095 in C57BL/6 mice and 230-1405 in BALB/c mice. Negligible neutralizing antibody responses were seen in mice immunized with an equivalent amount of purified monomeric RBD. Lederer et al. immunized mice with two doses of RBD mRNA and also reported neutralizing antibody titers two-logs higher than those for mice immunized with monomeric RBD protein (Lederer et al., 2020). Kang et al. reported a similar enhancement in immunogenicity for RBD-nanoparticle constructs compared to monomeric; RBD (Kang et al., 2021). The reciprocal of the dilution required for 50% neutralization for sera from mice immunized with the RBD-conjugated nanoparticles adjuvanted with AddaVax after the second boost were ~ 103 and these neutralizing titers were 10- 120- fold greater than those for sera from animals immunized with the monomeric RBD Thus, the present neutralizing antibody titers after a prime and boost (Table 3), which represent the reciprocal of the highest dilution that completely prevented cytopathic effects, compare well with these other RBD-based vaccine results against early isolates of SARS-CoV-2.

Recently, Saunders et al. immunized macaques with RBD-ferritin nanoparticle conjugates (RBD-scNP) and characterized neutralization efficacy against SARS-CoV-2 variants of concern. Two RBD-scNP immunizations induced potent serum nAbs with fifty percent inhibitory 1 reciprocal serum dilution neutralization titers ranging from 21,292 to 162,603. Neutralization was also reported against the variants B.1.1.7, B.1.351, 3 and P.1 While efficacy against B.1.617.2 (delta) was not reported, RBD-scNP sera would likely neutralize this variant, as was shown for RBD-SpyCatcher-mi3 (Table 5).

The present neutralizing antibody titers against the variants of concern were compared with those obtained from sera of immunized individuals Tada et al. compared the neutralization titers of serum antibodies from individuals immunized with three U.S. FDA Emergency use authorization vaccines (BNT162b2, mRNA-8 1273, and Ad26.COV2.S) using viruses pseudotyped with S proteins from SARS-CoV-2 variants (Tada et al.. 2021). BNT162b2. mRNA-1273, and AD26.COV2.S sera neutralized virus pseudotyped with the D614G spike protein with average neutralizing antibody half-maximal inhibitory concentration (IC₅₀) titers of 695, 833, and 221 respectively. Neutralizing titers were lower — 191, 208, and 30 respectively — for viruses pseudotyped with spike proteins from the Delta variant. Thus, our neutralizing antibody titers (Table 5) compare favorably with those obtained using these vaccine candidates that have been through clinical trials.

### Conclusions

A vaccine candidate consisting of the SpyCatcher-mi3 protein nanoparticle displaying the SARS-CoV-2 RBD was evaluated. The RBD-SpyCatcher-mi3 retained proper structure of binding epitopes following conjugation. This vaccine elicited very high levels of neutralizing antibodies in immunized mice against the original SARS-CoV-2 as well as three variants of concern. These studies strongly support the further testing of RBD-based vaccines for clinical use as a vaccine that elicits broadly neutralizing antibodies.

Stamatatos et al. (2020) recently reported that the immunization of those previously infected with SARS-CoV-2 can significantly boost neutralizing antibody titers against all variants, with the neutralization attributed to antibodies targeting the RBD. In light of the robust and broadly protective responses seen in naive mice by immunization with RBD-SpyCatcher-mi3, it MAY be interesting to characterize how the response is influenced by pre-existing immunity due to infection or immunization. With an increasing percentage of the population already infected or vaccinated, characterization of the role of pre-existing immunity will be an increasingly important consideration in vaccine design.

TABLE 5

Antibody responses to RBD-SpyCatcher-mi3 after prime and boost in mice
Spike IgG Endpoint Titer^{a}

| | S-614D | | P.1 | | B.1.1.7 | | B.1.351 | | SARS-CoV-1 | |
|---|---|---|---|---|---|---|---|---|---|---|
| Vaccine Group | Geometric Mean | Geometric SD Factor | Geometric Mean | Geometric SD Factor | Geometric Mean | Geometric SD Factor | Geometric Mean | Geometric SD Factor | Geometric Mean | Geometric SD Factor |
| SpyCatcher-mi3 | <20 | - | <20 | - | <20 | - | <20 | - | <20 | - |
| RBD-SpyCatcher-mi3 | 260,08 0 | 2.23 | 260,08 0 | 2.23 | 260,08 0 | 2.23 | 163,84 0 | 4.00 | 65,020 | 1.49 |

Neutralizing Antibody Titer^{b}

| | S-614D | P.1 | B.1.1.7 | B.1.351 | B.1.617.2 |
|---|---|---|---|---|---|
| Vaccine Group | Geometric Mean | Geometric Mean | Geometric Mean | Geometric Mean | Geometric Mean |
| SpyCatcher-mi3 | <20 | <20 | <20 | <20 | <20 |
| RBD-SpyCatcher-mi3 | 1016 | 5120 | 3225 | 640 | 508 |

^{a}Viral antibody endpoint titers against the SARS-CoV-2 and SARS-CoV-1 spike proteins (three animals in each group). Endpoint titers using 2-fold diluted sera were expressed as the reciprocal of the highest dilution with an optical density at 490 nm cutoff value >0.15; sera were collected on day 47 after the initial immunization.
^{b}Viral neutralization titers (three animals in each group). Endpoint titers using 2-fold diluted sera were expressed as the reciprocal of the highest dilution that completely prevented cytopathic effects; sera were collected on day 47 after the initial immunization.

### EXAMPLE 5

Exemplary coat proteins of a phage useful in the constructs, nanoparticles and methods include but are not limited to

masnftqfvl vdnggtgdvt vapsnfangv aewissnsrs qaykvtcsvr qssaqnrkyt
ikvevpkvat qtvggvelpv aawrsylnle ltipifatnp dcelivkamq gllkdgngpip
saiaansgiy (SEQ ID NO:10)

or

masnftqfvl vdnggtgdvt vapsnfangv aewissnsrs qaykvtcsvr qssaqnrkyt
ikvevpkvat qtvggvelpv aawrsylnle ltipifatnp dcelivkamq gllkdgnpip
saiaansgiy (SEQ ID NO:11)

or SEQ ID NO:1 or 4,
or a polypeptide having at least 80%, 82%, 85%, 86%, 88%, 90%, 92%, 94%, 95%, 97%, 98% or 99% amino acid sequence identity thereto.

An exemplary fibronectin binding protein useful in the constructs, nanoparticles and methods includes but is not limited to

mrraennkhs rysirklsvg vtsiaiaslf lgkvayavdg ippisltqkt tattsenwhh
idkdgliplg isleaakeef kkeveesrls eaqketykqk iktapdkdkl lftyhseymt
avkdlpaste sttqpveapv qetqasasds mvtgdstsvt tdspeetpss espvapalse
apaqpaesee psvaasseet pspstpaaps tpaapetpee paapsqpaes eessvaatts
pspstpaese tqtppavtkd sdkpssaaek paasslvseq tvqqptskrs sdkkeeqeqs
yspnrslsrq vrahesgkyl pstgekaqpl fiatmtlmsl fgsllvtkrq ketkk (SEQ ID NO:12)

or

mrraennkhs rysirklsvg vtsiaiaslf lgkvayavdg ippisltqkt tattsenwhh
idkdgliplg isleaakeef kkeveesrls eaqketykqk iktapdkdkl lftyhseymt
avkdlpaste sttqpveapv qetqasasds mvtgdstsvt tdspeetpss espvapalse
apaqpaesee psvaasseet pspstpaaps tpaapetpee paapsqpaes eessvaatts
pspstpaese tqtppavtkd sdkpssaaek paasslvseq tvqqptskrs sdkkeeqeqs
yspnrslsrq vrahesgkyl pstgekaqpl fiatmtlmsl fgsllvtkrq ketkk (SEQ ID NO:13)

or a polypeptide having at least 80%, 82%, 85%, 86%, 88%, 90%, 92%, 94%, 95%, 97%, 98% or 99% amino acid sequence identity thereto.

An exemplary SpyCatcher sequence useful in the constructs, nanoparticles and methods includes but is not limited to SEQ ID NO:7 or a polypeptide having at least 80%, 82%, 85%, 86%, 88%, 90%, 92%, 94%, 95%, 97%, 98% or 99% amino acid sequence identity thereto.

Exemplary S sequences useful in the constructs, nanoparticles and methods include but are not limited to SEQ ID NO:2, SEQ ID NO:3, SEQ ID NO:5, SEQ ID NO:6, SEQ ID NO:7, or a polypeptide having at least 80%, 82%, 85%, 86%, 88%, 90%, 92%, 94%, 95%, 97%, 98% or 99% amino acid sequence identity thereto.

### Example 6

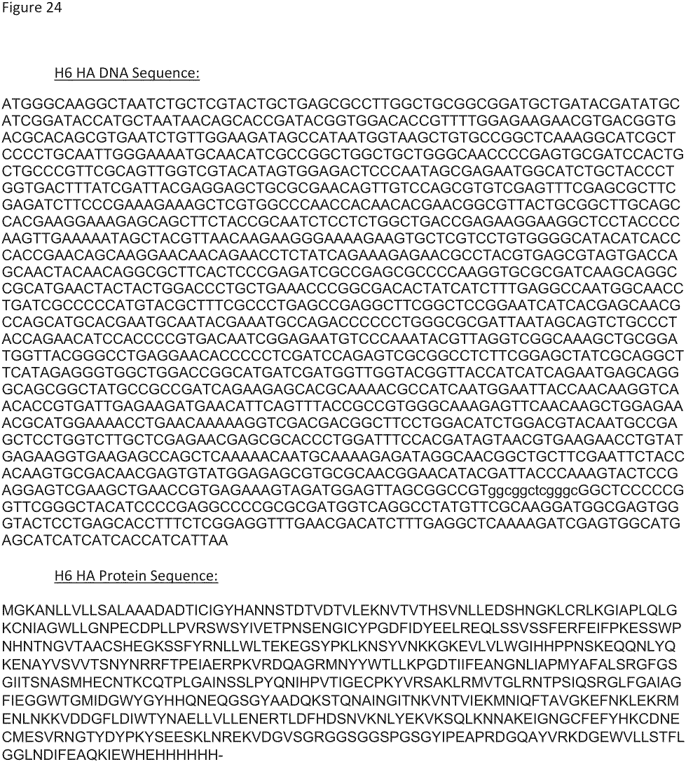

VLP-HA conjugates (normal HA orientation) that generate a robust and durable immune response in ferrets after a single immunization were prepared and tested (FIG. 20). Ferrets were immunized with VLP-HA conjugates or recombinant HA trimers. As seen in FIG. 20B, ferrets vaccinated with the VLP-HA scaffolds showed significantly enhanced neutralizing antibody titers after a single immunization, at a level that would be sufficient for protection. An experiment was performed to monitor the longevity of protection and found that enhanced neutralizing antibody titers were retained over a 40-week period even after a single immunization (FIG. 20C).

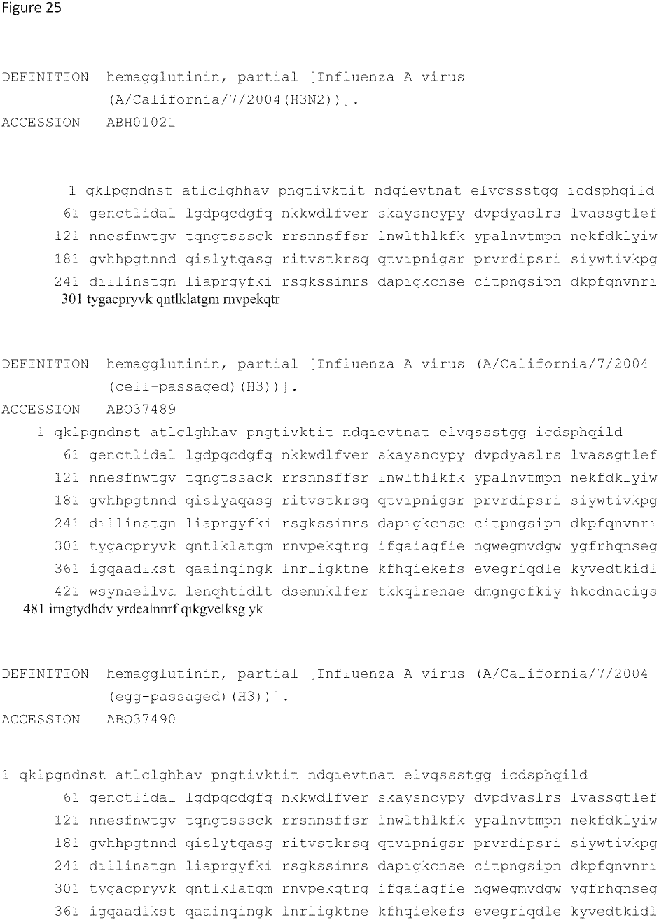

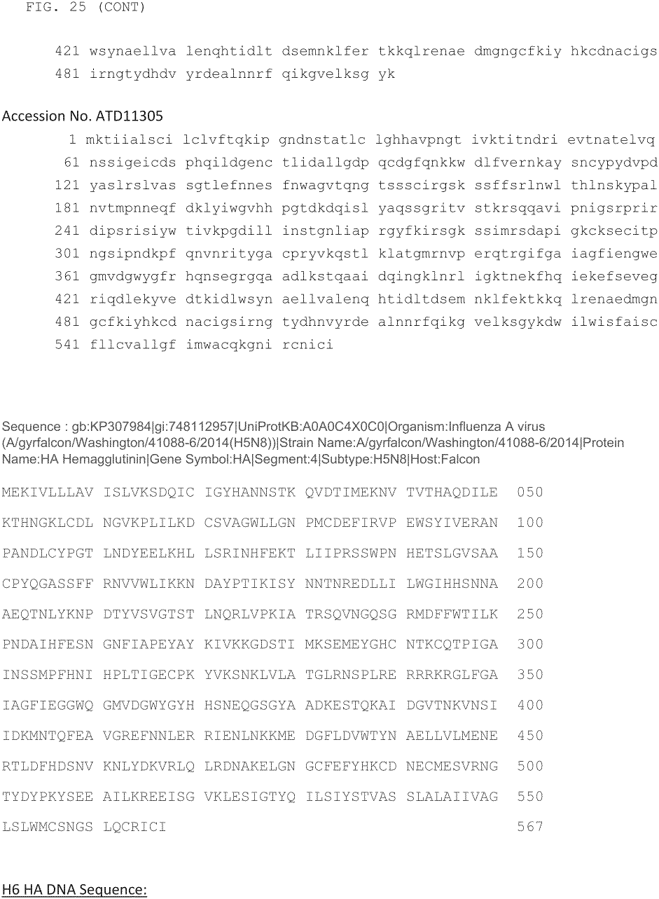

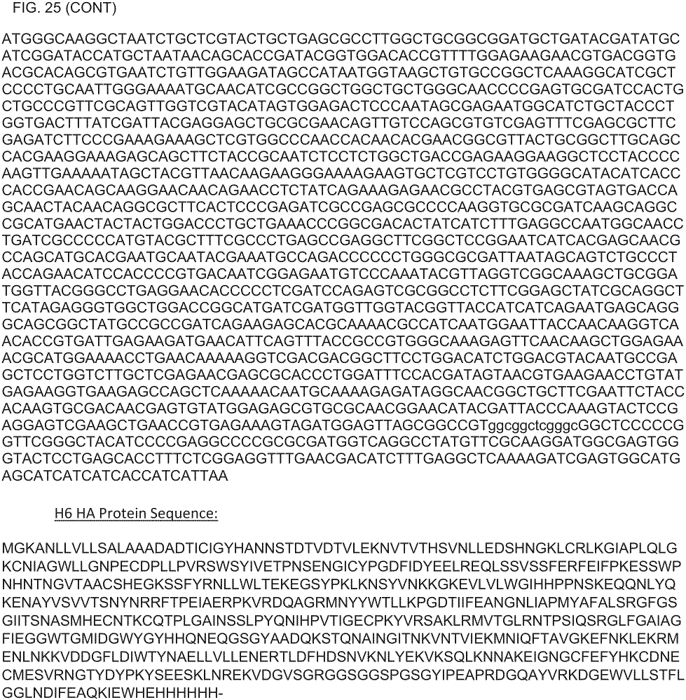

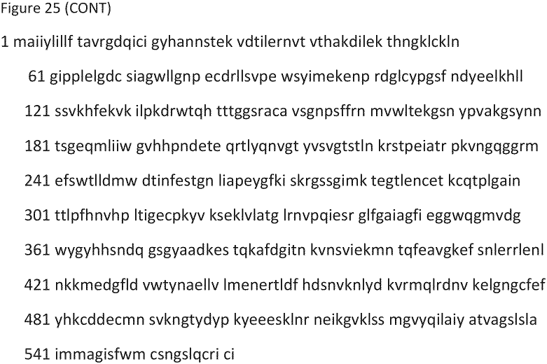

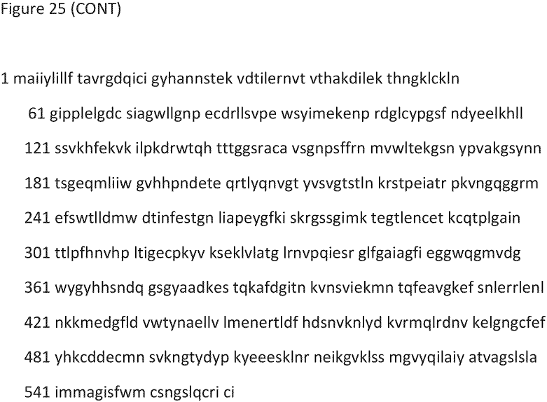

A reduced amount of HA-VLP conjugates was tested in a ferret immunization model. As seen in FIG. 23, the ferrets immunized with HA-VLP conjugate containing 22.5 ug, 11.25 ug, or 5.63 ug of HA (50%, 25%, or 12.5% of the amount used in FIG. 21) adjuvanted with AddaVax showed the equivalent levels of neutralization titers in sera against the homologous virus PR8 at 3 wks post immunization. No significant difference was found in the neutralization titers induced by subcutaneous wHA-VLP injection and by intramuscular injection (FIG. 23). The normal HA amount in influenza vaccines for human (15 ug) conjugated to VLP by an intramuscular route is expected to be sufficient to induce protective neutralization titers after a single immunization.

### Example 7

### Methods

### Expression and Purification of SARS-CoV-2 S2 and S2_{mutS2′} proteins

The gene encoding the S2 subunit of the SARS-COV-2 HexaPro (Hsieh et al., 2020) spike protein (residues 686 to 1208) with an N-terminal mouse lg Kappa signal peptide and C-terminal T4 fibritin trimerization 4 domain, AviTag, and his-tag was cloned into pcDNA3.1 between the Ncol and Xhol restriction sites 5 by Gene Universal, Inc. (Newark DE). The S2_{mutS2′} variant was created such that S2 residues 814 and 815 were mutated to glycine residues to eliminate the S2′ protease cut site. These plasmids were transfected into Expi293F cells using the ExpiFectamine Transfection Kit (Thermo Fisher Scientific) and associated protocol. The cells were incubated for 5 days, after which the cultures were centrifuged at 5,500xg for 20 minutes. The supernatant was dialyzed into PBS and then was allowed to flow through 1 mL of of HisPur Ni-NTA resin (Thermo Scientific) in a gravity flow column (G-Biosciences) that had been washed with DI water and pre-equilibrated with phosphate-buffered saline (PBS). The column was then washed with 90 column volumes of wash buffer (42 mM sodium bicarbonate, 8 mM sodium carbonate, 300 mM NaCl, 20 mM imidazole). The protein was eluted by incubating the resin in 3 mL of elution buffer (42 mM sodium bicarbonate, 8 mM sodium carbonate, 300 mM NaCl, 300 mM imidazole) for 5 minutes before allowing the elution buffer to flow through the column. The eluate was collected. This elution procedure was repeated twice more such that a total of 9 mL of eluate was collected. The eluate was buffer exchanged into 20 mM Tris, 20 mM NaCl, pH 8.0, to prepare for in vitro biotinylation. The concentration of the protein solutions was quantified using the BCA assay (Thermo Scientific).

### Expression and Purification of MS2

The following protocol regarding the expression and purification of MS2 has been previously described (Chiba et al., 2020). The DNA sequence corresponding to a single chain dimer of MS2 coat protein with an AviTag inserted between the fourteenth and fifteenth residues of the first coat protein monomer was cloned into pET-28b between the Ndel and Xhol restriction sites by GenScript Biotech Corporation (Piscataway, NJ). This plasmid and a plasmid coding for pAcm-BirA (Avidity LLC) were cotransformed into BL21(DE3) Escherichia coli (E. coli) (New England BioLabs). The transformation was added to 5 mL of 2xYT that had been supplemented with kanamycin and chloramphenicol. This small culture was incubated in a shaking incubator overnight at 37° C. The following morning, the 5 mL culture was added to 1 L of 2xYT that had been supplemented with 4 kanamycin and chloramphenicol. The 1 L culture was placed in a shaking incubator at 37° C. Once the culture’s optical density reached 0.6, expression of the MS2 and BirA was induced with IPTG (1 mM; GoldBio). The culture was also supplemented with approximately 12.5 µg of biotin, and remained shaking in the incubator overnight at 30° C. After the overnight expression, the culture was centrifuged at 7000×g for 7 minutes to pellet the cells. The cell pellet was then homogenized into 25 mL of 20 mM Tris buffer (pH 9.0) supplemented with lysozyme (0.5 mg/mL; Alfa Aesar), a protease inhibitor tablet (Sigma-Aldrich), and benzonase (125 units; EMD Millipore). The resulting cell suspension was kept on ice for 20 minutes while occasionally mixing. Next, sodium deoxycholate was added to a final concentration of 0.1% (w/v). The cells were kept on ice and sonicated for 3 minutes at an amplitude of 35% with 3 second pulses (Sonifier S-450, Branson Ultrasonics). This sonication procedure was repeated after allowing the cells to cool on ice for at least 2 minutes. The resulting lysate was centrifuged at 27,000×g for 30 minutes. The supernatant was collected and was centrifuged again at 12,000×g for 15 minutes. The supernatant resulting from the second centrifugation was diluted 3-fold with 20 mM Tris, pH 8.0, and filtered using a 0.45 µm bottle-top filter. The filtrate was then run through four HiScreen Capto Core 700 columns (Cytiva) in parallel according to the manufacturer’s operating instructions, resulting in fractions that contained MS2. The fractions were run on an SDS-PAGE gel to assess MS2 purity and recovery. Fractions containing pure MS2 were pooled, concentrated using a 10 kDa MWCO centrifugal filter (Millipore Sigma), and further purified using a Superdex 200 increase 10/300 SEC column (Cytiva). The SEC fractions containing MS2 were pooled and buffer exchanged into 20 mM Tris, 20 mM 24 NaCl, pH 8.0, in preparation for in vitro biotinylation. MS2 was quantified using the BCA assay (Thermo Scientific).

### Expression, Refolding, and Purification of Streptavidin (SA)

The following protocol regarding the expression, refolding, and purification of SA has been previously described and was adapted from methods documented by Fairhead et al. and Howarth et al. (Chiba et al., 2020; Fairhead et al., 2014; Howarth & Ting, 2008). A plasmid encoding SA (Addgene plasmid #46367, a gift from Mark Howarth) was transformed into BL21 (DE3) E. coli. The transformation was added to 5 mL of 2×YT supplemented with ampicillin, and this small culture was grown overnight in a shaking incubator at 37° C. The next morning the culture was added to four, 1 L shake flasks of 2×YT supplemented with ampicillin. These larger cultures were placed in a shaking incubator at 37° C. until the cultures’ OD reached 0.6, at which point the expression of streptavidin as inclusion bodies was induced with IPTG (1 mM; GoldBio), and the temperature of the incubator was reduced to 30° C. After overnight incubation, the cultures were centrifuged at 7,000×g for 15 minutes such that there were two cell pellets. The two resulting cell pellets were each homogenized into 50 mL of resuspension buffer (50 mM Tris, 100 mM NaCl, pH 8.0) supplemented with lysozyme (1 mg/mL; Alfa Aesar) and benzonase (500 units; EMD Millipore). The homogenized cells were incubated at 4° C. for at least 30 minutes. After this incubation step, the cells were further homogenized and sodium deoxycholate was added to a final concentration of 0.1% (w/v) before sonicating (Sonifier S-450, Branson Ultrasonics) for 3 minutes with 3 second pulses at 35% amplitude. The lysed cells were then centrifuged at 27,000×g for 15 minutes. The supernatant was discarded, and the lysis procedure was repeated. When the lysis step was repeated the incubation time at 4° C. prior to sonication was reduced to 15 minutes. After the lysis procedure had been performed twice, the two resulting inclusion body pellets were each suspended in 50 mL wash buffer (50 mM Tris, 100 mM NaCl, 100 mM EDTA, 0.5% (v/v) Triton X-100, pH 8.0), homogenized, sonicated for 30 seconds at an amplitude of 35%, and centrifuged at 27,000×g for 15 minutes. This wash procedure was repeated twice more. The resulting inclusion body pellets were then suspended in 50 mL of a second wash buffer (50 mM Tris, 10 mM EDTA, pH 8.0), homogenized, sonicated for 30 seconds at an amplitude of 35%, and centrifuged at 15,000×g for 15 minutes. This second wash step was performed twice. The washed inclusion body pellets were then unfolded by being homogenized into 10 mL of a 7.12 M guanidine hydrochloride solution. This solution of unfolded streptavidin in guanidine hydrochloride was stirred at room temperature for an hour, after which it was centrifuged at 12,000×g for 10 minutes. The supernatant was then added dropwise at a rate of 30 mL/h to 1 L of chilled PBS that was being stirred vigorously. This rapid dilution of the streptavidin and guanidine hydrochloride allowed for the streptavidin to fold properly. The folded streptavidin in PBS was stirred overnight at 4° C., and was then centrifuged at 7,000×g for 15 minutes to remove insoluble protein. The supernatant was filtered using a 0.45 µm bottle-top filter, and was then stirred while ammonium sulfate was slowly added to a concentration of 1.9 M. This concentration of ammonium sulfate serves to precipitate out impurities. The solution was stirred for at least 3 hours at 4° C., after which it was centrifuged at 7,000×g for 15 minutes to pellet the precipitated impurities. The supernatant was filtered using a 0.45 µm bottle-top filter, and was then stirred while ammonium sulfate was added to a total concentration of 3.68 M. This concentration of ammonium sulfate precipitates the streptavidin. The solution was stirred for at least 3 hours at 4° C. before being centrifuged at 7,000×g for 20 minutes to pellet the streptavidin. The supernatant was discarded, and the pelleted streptavidin was suspended in 20 mL of Iminobiotin Affinity Chromatography (IBAC) binding buffer (50 mM Sodium Borate, 300 mM NaCl, pH 11.0). This streptavidin solution was then allowed to flow through 5 mL of Pierce Iminobiotin Agarose (Thermo Scientific) in a gravity flow column (G-Biosciences) that had been rinsed with DI water and pre-equilibrated with IBAC binding buffer. The column was next washed with column volumes of IBAC binding buffer, and the streptavidin was eluted from the column with 6 column volumes of elution buffer (20 mM KH₂PO₄, pH 2.2). The eluate was collected, dialyzed into PBS, and concentrated using a 10 kDa MWCO centrifugal filter (Millipore Sigma). The concentration of streptavidin was quantified by measuring the UV absorption at 280 nm.

### Expression and Purification of 0304-3H3 Antibody

The genes encoding the variable regions of the heavy chain and light chain of the 0304-3H3 antibody (Chi et al., 2020) were cloned into the TGEX-HC and TGEX-LC vectors (Antibody Design Labs), respectively, by Gene Universal, Inc. (Newark, DE). The plasmids were co-transfected in a 2:1 light chain to heavy chain ratio into Expi293F cells using the ExpiFectamine Transfection Kit (Thermo Fisher Scientific) and associated protocol. After a 4-day incubation, the culture was centrifuged at 5,500×g for 20 minutes. The supernatant was diluted in PBS and filtered before being purified by using a 1 mL MabSelect SuRe column (Cytiva) according to the manufacturer’s protocol. The concentration of the purified 0304-3H3 antibody was quantified using the BCA assay (Thermo Scientific).

### In Vitro Biotinylation of AviTagged Proteins

The BirA-500 kit (Avidity LLC) and general protocol were used to biotinylate the AviTagged MS2 and S2 proteins. In brief, the proteins were buffer exchanged into 20 mM Tris, 20 mM NaCl, pH 8.0. The concentration of protein in solution was adjusted to either 45 µM for MS2 or 15 µM for S2 and S2mutS2′ before adding the recommended amount of Biomix B (a proprietary mixture of biotin, ATP, and magnesium acetate). The recommended amount of BirA was added to the MS2 solution, while three times the recommended amount of BirA was added to the S2 solutions. These solutions were incubated at 37° C. for 2 hours while shaking vigorously. After the two-hour incubation, the solutions were moved to a nutator at 4° C. for overnight incubation. Finally, the biotinylated proteins were separated from the biotinylation reagents using a Superdex 200 increase 10/300 column (Cytiva) and quantified by using the BCA assay (Thermo Scientific).

### Assembly of MS2-SA VLP

The assembly of MS2-SA VLP has been previously described (Chiba et al., 2020). Approximately 1 mL of biotinylated MS2 at a concentration of about 0.7 mg/mL was added 2.5 µL at a time to stirred streptavidin that was in approximately 20-times molar excess and at a concentration of around 60 mg/mL. This mixture was stirred for 30 minutes at room temperature before the MS2-SA VLP was separated from excess streptavidin using a Superdex 200 increase 10/300 column (Cytiva). To quantify the purified MS2-SA VLP, a small sample of the MS2-SA VLP in Nu-PAGE lithium dodecyl sulfate (LDS) sample buffer (Invitrogen) was heated at 90° C. for at least 10 minutes and run on an SDS-PAGE gel with heated streptavidin standards of known mass.

### Assembly of VLP-S2 and VLP-S2_{mutS2′}

MS2-SA and biotinylated S2 or S2_{mutS2′} were mixed in a ratio determined using analytical SEC. Mixtures consisting of 5 µg of S2 or S2_{mutS2′} and varying amounts of MS2-SA were run through a Superdex 200 increase 10/300 SEC column (Cytiva). The ratio of the mixture with the least amount of MS2-SA that resulted in a chromatogram without a peak corresponding to excess S2 or S2_{mutS2′} was the stoichiometric ratio used to generate VLP-S2 and VLP-S2_{mutS2′} for characterization and immunization.

### Sds-page

Protein samples were diluted with 5 µL of Nu-PAGE lithium dodecyl sulfate (LDS) sample buffer (Invitrogen). These protein samples and PageRuler Plus Prestained Protein Ladder (Thermo Scientific) were loaded into the wells of a 4-12% Bis-Tris gel (Invitrogen). The gel was run in MES-SDS buffer at 110 V for 60 minutes while being chilled at 4° C. The gel was stained with SimplyBlue SafeStain (Invitrogen), destained, and imaged using the ChemiDoc MP imaging system (Bio-Rad).

Characterization of S2, S2_{mutS2′}, VLP-S2, and VLP-S2_{mutS2′} by ELISA Antigen (0.1 µg S2 and S2mutS2′ -alone and on VLP) was coated onto Nunc MaxiSorp 96-well flat-bottom plates (Invitrogen). The antigen solution was incubated for 1 hour, before the wells were emptied and 5% BSA (Millipore) in PBST (PBS with 0.05% Tween-20) was added to the wells. This BSA solution remained in the wells for 45 minutes, after which it was discarded from the plate and each well was washed with 200 µL of PBST three times. Next, primary antibody (0304-3H3) was diluted in 1 % BSA in PBST and a final volume of 100 µL was added to each well. The moles of antibody per well were equivalent to the moles of S2 trimer that had been coated in the well. The plate was left to incubate with the primary antibody for an hour, after which the plate was emptied, and each well was washed with 200 µL of PBST three times. Then 100 µL of the secondary antibody, horseradish peroxidase-conjugated anti-human IgG Fc fragment antibody (MP Biomedicals; 1:5,000 dilution) in 1 % BSA in PBST was added to each well. The secondary antibody solution remained in the plate for 1 hour, after which the solution was discarded, and the wells of the plate were washed with 200 µL of PBST three times. The plate was then developed by adding 100 µL of TMB substrate solution (Millipore) to each well. The reaction was stopped after three minutes by adding 0.16 M sulfuric acid to each well. The absorbance of each well was then read at 450 nm using a Spectramax i3x plate reader (Molecular Devices).

### Dls

MS2-SA VLP was diluted in PBS to 100 µL such that there was 1 µg of SA in solution. VLP-S2 and VLP-S2_{mutS2′} were each diluted in PBS to 100 µL such that there was 5 µg of S2 in solution. Each 100 µL solution was then pipetted into a UVette (Eppendorf), which was inserted into a DynaPro NanoStar Dynamic Light Scattering detector (Wyatt Technology). Dynamics software (Wyatt Technology) brought the temperature of the measurement cell to 25° C. The detector then proceeded with the measurement. Each measurement was the result of 10 acquisitions and was output as % Intensity, which could be converted to % Mass using the Isotropic Spheres model.

### Negative Stain Transmission Electron Microscopy

Conventional native-stain transmission electron microscopy (TEM) was performed, as described previously (Booth et al., 2011). Briefly, 4 µl of diluted samples were applied onto glow-discharged mesh copper grids (CF300-Cu; Electron Microscopy Science, PA), washed with PBS (1X), stained in droplets of 1 % phosphotungstic acid (PTA, PH 6~7) for 1 minute. The grids were then blotted from the grid backside and air-dried inside a petri dish for at least 30 minutes under room temperature to minimize the negative-stain artifacts of flattening and stacking (Jung & Mun, 2018). The negative-stain grids were imaged in low-dose mode (50 e-/Å), using a Talos L120C transmission electron microscope (Thermo Fisher 20 Scientific, previously FEI, Hillsboro, OR) at 120 kV, images were acquired on a 4k × 4k Ceta CMOS camera microscope (Thermo Fisher Scientific), using SerialEM 3.8 (Mastronarde, 2005).

### Plunge Freezing and Cryo-electron Microscopy

4 µl of the VLP suspension was added to a glow discharged copper grid (C-Flat 1.2/1.3, 400 mesh, Protochips) with an extra layer of carbon (about 2 nm) on the holey carbon surface. Grids were plunge frozen using a Vitrobot Mark IV (ThermoScientific) and stored in liquid nitrogen until imaging. Cryo-electron microscopy (cryo-EM) imaging was performed on a Titan Krios (ThermoScientific Hillsboro, OR, USA) operated at 300 kV. Images (defocus of -2~5 µm) were recorded on a post-GIF Gatan K3 camera in EFTEM mode (2.176 Å/pixel) with a 20-eV slit, CDS counting mode, using 3 SerialEM 3.8 (Mastronarde, 2005). A total dose of 30-40 e/Å2 was used and 40 frames were saved (about 1.2 e/Å² per frame). Frames were motion-corrected in MotionCor2 (Zheng et al., 2017). Images were low pass filtered to Å² for 5 better visualization and contrast using EMAN2 (Galaz-Montoya, 2015).

### Cells and Virus

Vero E6/TMPRSS2 cells obtained from the National Institute of Infectious Diseases, Japan (Imai et al., 2020) were maintained in high glucose Dulbecco’s modified Eagle’s medium (DMEM) containing 10% fetal bovine serum (FBS) and antibiotic/antimycotic solution along with G418 (1 mg/ml). Virus stocks were propagated and tittered on Vero E6 TMPRSS2 cells. The following challenge viruses were used in the hamster studies: SARS-CoV-2/UT-NCGM02/Human/2020/Tokyo (NCGM02), hCoV-12 19/USA/WI-UW-5250/2021 (B.1.617.2, delta), hCoV-19/USA/MD-HP01542/2021 (B.1.351, beta), hCoV-19/USA/WI-WSLH-221686/2021 (B.1.1.529 BA.1, Omicron), and Pg CoV, BetaCoV/pangolin/Guandong/1/2019.

### Animal Studies

Animals were housed for five days before the start of the study in rooms with controlled temperature and humidity along with a 12-hour light and dark cycle. Food and water were available ad libitum along with enrichment. Animals were monitored at least twice daily by trained personnel. Animals were randomly assigned to infection groups and researchers were not blinded on the selection of animals. Samples sizes of three or four hamsters and four to five mice were determined based on prior in vivo virus challenge experiments; no sample size calculations were performed to power each study. No animals were excluded, and all data was included in the analysis.

Wild-type Syrian hamsters (females; 4-5 weeks old; Envigo) were immunized with 20 µg of SARS-CoV-2 S2 protein presented on VLPs, a mutant S2 protein presented on VLPs, or VLPs without the S2 protein, by subcutaneous inoculation. One of the following adjuvants were added to each vaccine preparation before inoculation: AddaVax (InvivoGen; equal volume vaccine and adjuvant), QS-21 (Desert King; 25 µg), R848 (InvivoGen, 25 µg) and AddaS03 + pIC (InvivoGen equal volume of AddaS03 plus 100 µg of pIC). Animals were infected by intranasal inoculation with 10³ plaque-forming units (pfu) of SARS-CoV-2 while under isoflurane anesthesia. Three days after infection, animals were humanely sacrificed by overdose of isoflurane, and lung tissue and nasal turbinate samples were collected to measure amount of virus.

Wild-type BALB/c mice (females; 8-10 weeks old; Taconic Biosciences) were immunized by subcutaneous inoculation with 14 µg of SARS-CoV-2 S2 protein presented on VLPs or VLPs without the S2 protein, adjuvanted with AddaS03 + pIC. Four weeks after a single immunization, serum was collected from a group of animals, while another group of animals were infected by intranasal inoculation with 2 × 10³ pfu of mouse-adapted SARS-CoV-2⁴⁶ while under isoflurane anesthesia. Three days after infection, animals were humanely sacrificed by overdose of isoflurane, and lung tissue samples were collected to measure amount of virus.

Virus titers in the tissues were determined on confluent Vero E6/TMPRSS2 cells by infecting cells with 100 µl of undiluted or 10-fold dilutions (10⁻¹ to 10⁻⁵) of clarified lung and nasal turbinate homogenates. After a 30-minute incubation, the inoculum was removed, the cells were washed once, and then overlaid with 1% methylcellulose solution in DMEM with 5% FBS. The plates were incubated for three days, and then the cells were fixed and stained with 20% methanol and crystal violet in order to count the plaques.

### Detection of Antibodies Against the SARS-CoV-2 S2 in Immunized Hamsters.

ELISAs were performed using recombinant spike SARS-CoV-2 proteins either produced in Expi293F cells (Thermo Fisher Scientific) and then C-terminal His-tag purified by using TALON metal affinity resin (Wuhan, B.1.351, B.1.617.2, B.1.1.529 BA.1 and BA.2, RsSCH014, and Pg-CoV spike antigens) or purchased from Sino Biological (229E, OC43, HKU-1, NL63, and CoV-1 11 strain Tor2 spike antigens). ELISA plates were coated overnight at 4° C. with 50 µl of spike antigen at a concentration of 2 µg/ml in PBS. After blocking with PBS containing 0.1% Tween 20 (PBS-T) and 3% milk powder, the plates with incubated in duplicate with heat-inactivated serum diluted in PBS-T with 1% milk powder. A hamster IgG secondary antibody conjugated with horseradish peroxidase (Invitrogen; 1:7,000 dilution) was used for detection. Plates were developed with SigmaFast o-phenylenediamine dihydrochloride solution (Sigma), and the reaction was stopped with the addition of 3 M hydrochloric acid. The absorbance was measured at a wavelength of 490 nm (OD₄₉₀). Background absorbance measurements from serum collected before immunization was subtracted from serum collected before challenge for each dilution. IgG antibody endpoint titers were defined as the highest serum dilution with an OD₄₉₀ cut-off value of 0.15.

### Focus Reduction Neutralization Test (FRNT).

Neutralization of SARS-CoV-2 was characterized by using a focus reduction neutralization test. Serial dilutions of serum from vaccinated hamsters starting at a final concentration of 1:20 were mixed with about 2000 focus-forming units (FFU) of virus (NCGM02)/well and incubated for 1 hour at 37° C. Pooled serum from hamsters vaccinated with VLP without the S2 protein served as a control. The antibody-virus mixture was inoculated onto Vero E6/TMPRSS2 cells in 96-well plates and incubated for 1 hour at 37° C. An equal volume of methylcellulose solution was added to each well. The cells were incubated for 16 hours at 37° C. and then fixed with formalin. After the formalin was removed, the cells were immunostained with a mouse monoclonal antibody against SARS-CoV-½ nucleoprotein [clone 1C7C7 (Sigma-Aldrich)], followed by a horseradish peroxidase-labeled goat anti-mouse immunoglobulin (SeraCare Life Sciences). The infected cells were stained with TrueBlue Substrate (SeraCare Life Sciences) and then washed with distilled water. After cell drying, the focus numbers were quantified by using an ImmunoSpot S6 Analyzer, ImmunoCapture software, and BioSpot software (Cellular Technology). The results are expressed as the 50% focus reduction neutralization titer (FRNT50). The FRNT50 values were calculated by using Prism 9 (Graphpad Software). Percent neutralization was calculated as 100×(1 - [ratio of foci in the presence of sera from hamsters vaccinated with VLP-S2_{mutS2′} and foci in the presence of pooled sera from hamsters vaccinated with VLP control]). The FRNT₅₀ value was then calculated from the normalized percent neutralization using a four-parameter nonlinear regression in Graphpad Prism.

### Statistics

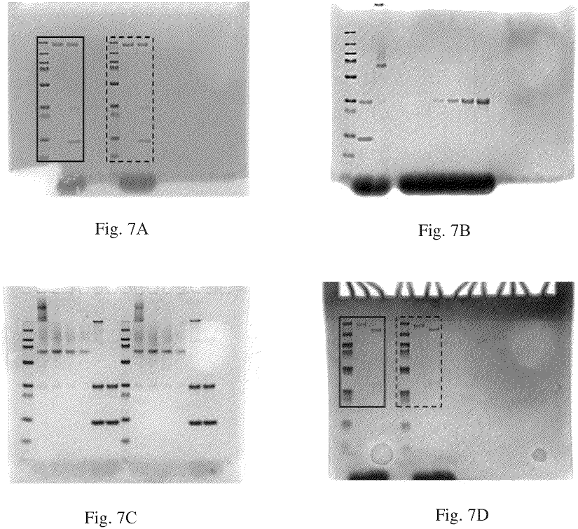

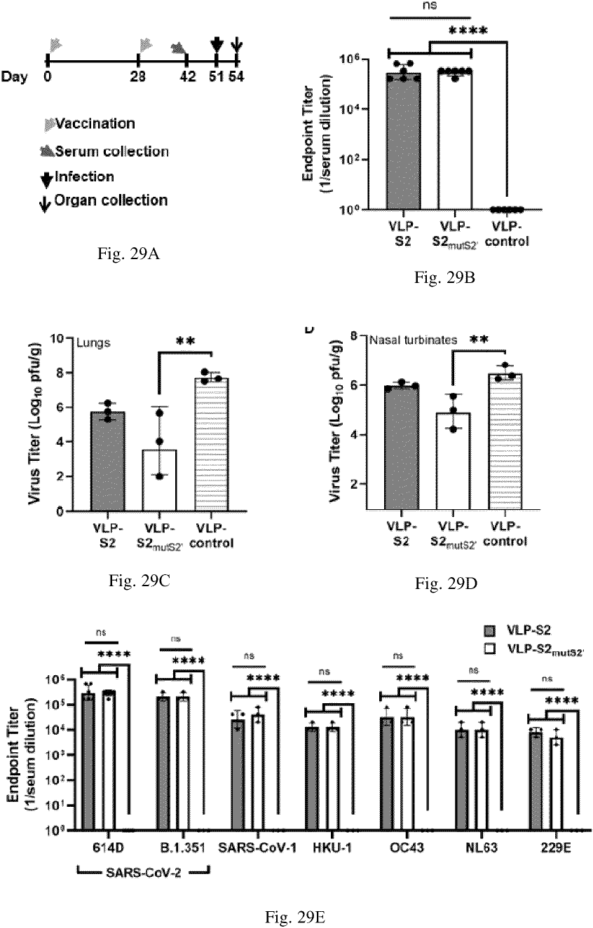

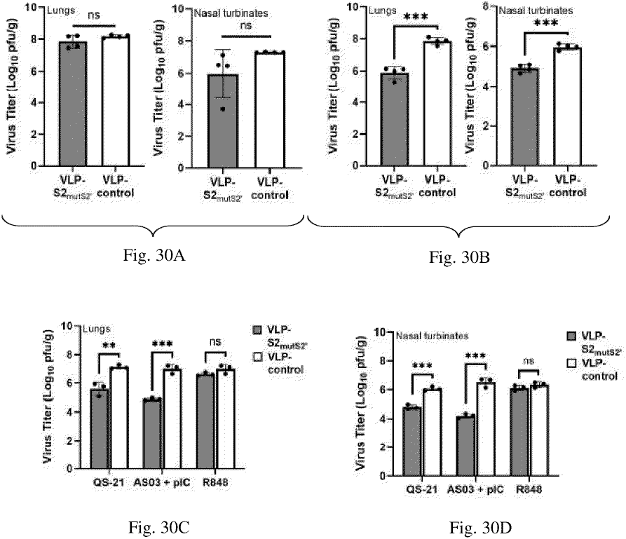

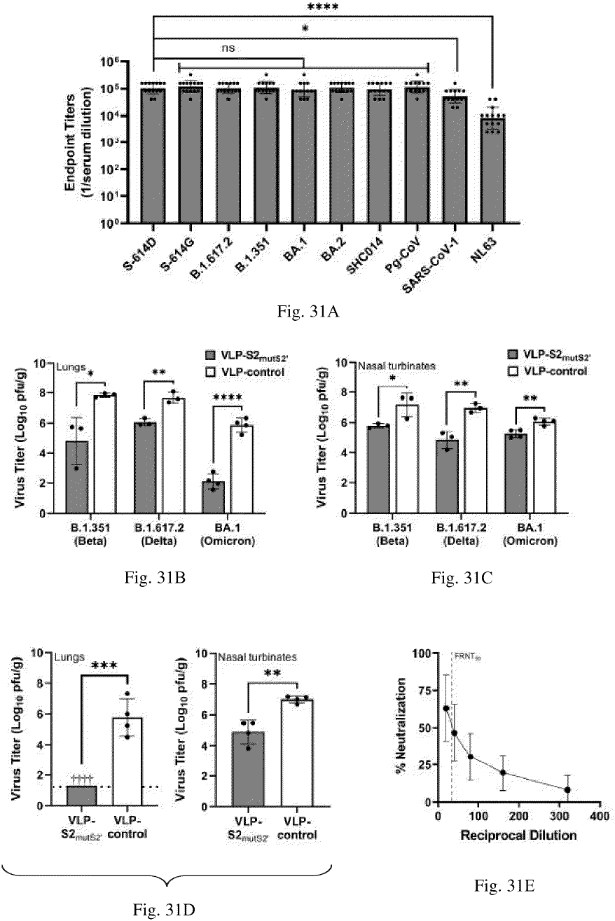

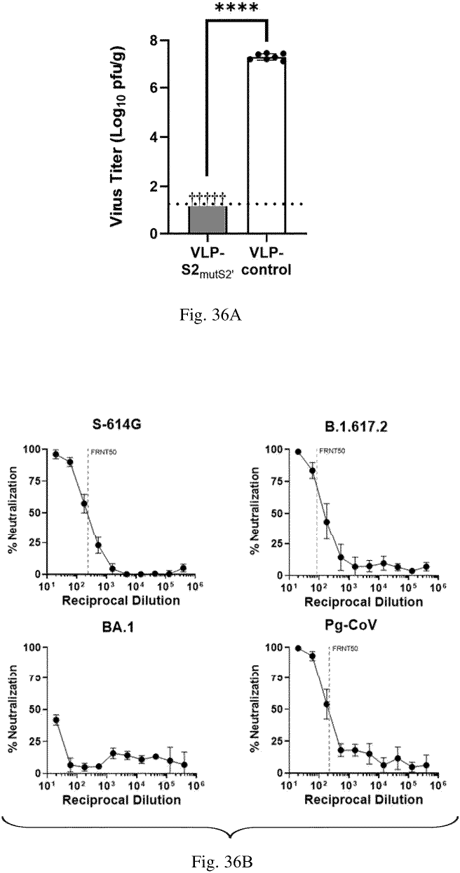

In vitro characterizations of the binding of 0304-3H3 to VLP-S2 and VLP-S2_{mutS2′} using ELISA were each conducted twice independently with three technical replicates for each condition. The data are presented as the mean ± SD. For in vivo characterization of VLP-S2 and VLP-S2_{mutS2′}, there were three groups (receiving either VLP-S2, VLP-S2_{mutS2′}, or VLP-control) each with three hamsters (n=3). To determine the resulting endpoint titers against the SARS-CoV-2 spike protein (FIG. 29b. Table 6), two independent assays were conducted using sera from each hamster. Significance was determined by a one-way analysis of variance (ANOVA) and Tukey post-hoc multiple comparison between groups (α = 0.05). For further in vivo characterization of VLP-S2_{mutS2′}, studies were conducted with two groups each (receiving either VLP-S2_{mutS2′} or VLP-2 control). Endpoint titers (FIG. 29e; FIG. 31a) were determined by conducting an assay using sera from each hamster (3 hamsters for FIG. 4e; 14 hamsters for FIG. 31a). The data are presented as the geometric mean with the geometric SD factor and significance was determined by a one-way analysis of variance (ANOVA) and Tukey post-hoc multiple comparison between groups (α = 0.05). Viral titers in the lungs and nasal turbinates of hamsters immunized with either VLP-S2, VLP-7 S2_{mutS2′}, or VLP-control 3 days after SARS-CoV-2 infection (FIGS. 29c, d) were presented as the mean with SD (n=3) and the significance was determined by a one-way analysis of variance (ANOVA) and Dunnett post-hoc multiple comparison between groups (α = 0.05). For all tests of significance, assumptions of the normality of residuals and homogeneity of variance were validated by the D′Agostino-Pearson test and the Brown-Forsythe test, respectively. Viral titers in the lungs and nasal turbinates of hamsters immunized with either VLP-S2_{mutS2′} or VLP-control 3 days after infection with a SARS-CoV-2 variants or Pg-CoV (FIG. 30, FIGS. 31a-d) were presented as the mean with SD (n=3 or 4) and the significance was determined by either unpaired t-test or Welch’s t-test. Three hamsters per group were used for the study presented in FIGS. 5a-b and four hamsters per group for the study in FIGS. 5c-d. For the study in FIG. 6, three hamsters per group were challenged with either B.1.351 or B.1.617.2, and four hamsters per group were challenged with either BA.1 or Pang-CoV. For all measurements of viral titers, sera from each hamster was used in a single assay. For all tests of significance, assumption of the normality of residuals was validated by the Shapiro-Wilk and Kolmogorov-Smirnov tests. The homogeneity of variances was determined by the F-test of equality of variances. For the study presented in FIG. 36, five mice were immunized with VLP-S2 and seven mice were immunized with VLP-control. For the measurement of lung viral titers, a single assay was conducting using sera from each mouse. Lung viral titers were presented as the mean with SD (n = 5 of 7) and the significance was determined by Welch’s t-test. Percent neutralization in FIGS. 6e and 7b were presented as mean with SD. A single assay was conducted using sera from each animal (n = 14 hamsters for FIG. 6e. n = 3 for BA.1 in FIG. 36b, and n = 4 for FIG. 36b other coronaviruses). All statistical analysis was carried out using Prism 9 (GraphPad).

TABLE 6

Antibody responses to VLP-S2 and VLP-S2_{mutS2′} after prime and boost in Syrian hamsters
Spike IgG Endpoint Titer

| | SARS-Cov-2 | | | | SARS-CoV-1 | | HKU-1 | | OC43 | | NL63 | | 229E | |
|---|---|---|---|---|---|---|---|---|---|---|---|---|---|---|
| | 614D | | B.1.351 | | | | | | | | | | | |
| Vaccine Group | Geometric mean | Geometric SD Factor | Geometric mean | Geometric SD factor | Geometric mean | Geomtric SD factor | Geometric mean | Geometric SD factor | Geometric mean | Geometric SD factor | Geometric mean | Geometric SD factor | Geometric mean | Geometric SD factor |
| MS2-SA VLP | <20 | - | <20 | - | <20 | - | <20 | | <20 | - | <20 | - | <20 | - |
| VLP-S2 | 292.667 | 1.98 | 206.425 | 1.49 | 25.803 | 2.23 | 12.902 | 1.49 | 32.404 | 2.23 | 10.240 | 2.00 | 8127 | 1.49 |
| VLP-S2_{mutS2′} | 291.930 | 1.33 | 206.425 | 1.49 | 40.960 | 2.00 | 12.902 | 1.49 | 32.510 | 2.23 | 10.240 | 2.00 | 5120 | 2.00 |

Viral antibody endpoint titers against the SARS-CoV-2 spike (three animals in each group). Endpoint titers using 2-fold diluted sera were expressed as the reciprocal of the highest dilution with an optical density at 490
nm cutoff value > 0.15; sera were cikkected on day 42 after the initial immunization.

### Part A: Results and Discussion

### Generation and in Vitro Characterization of S2 Nanoparticle-based Vaccines

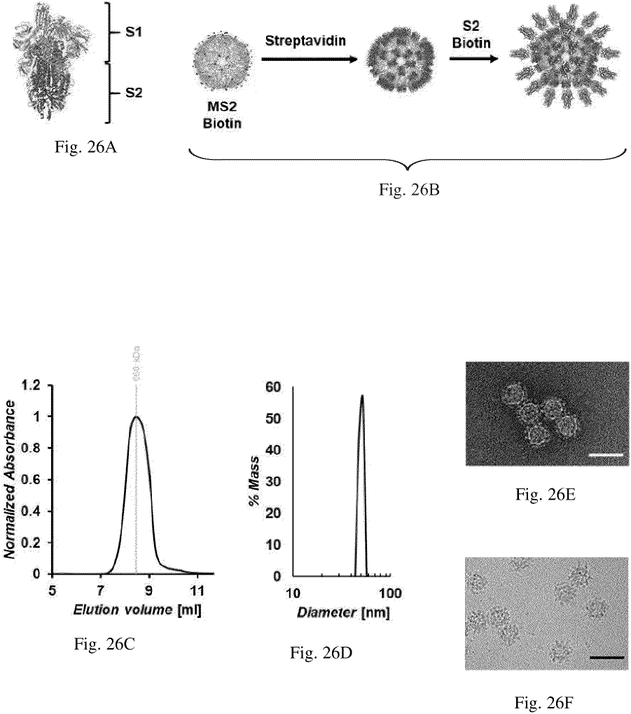

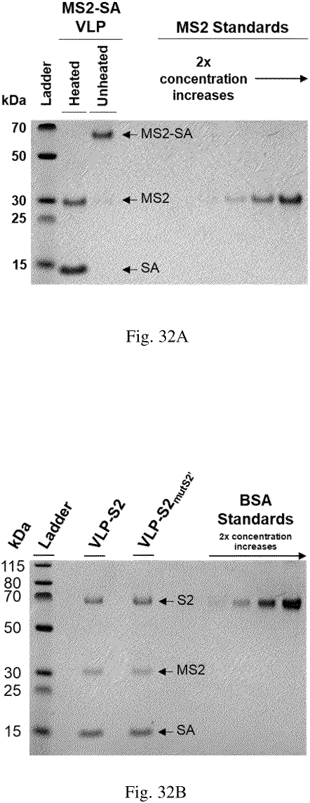

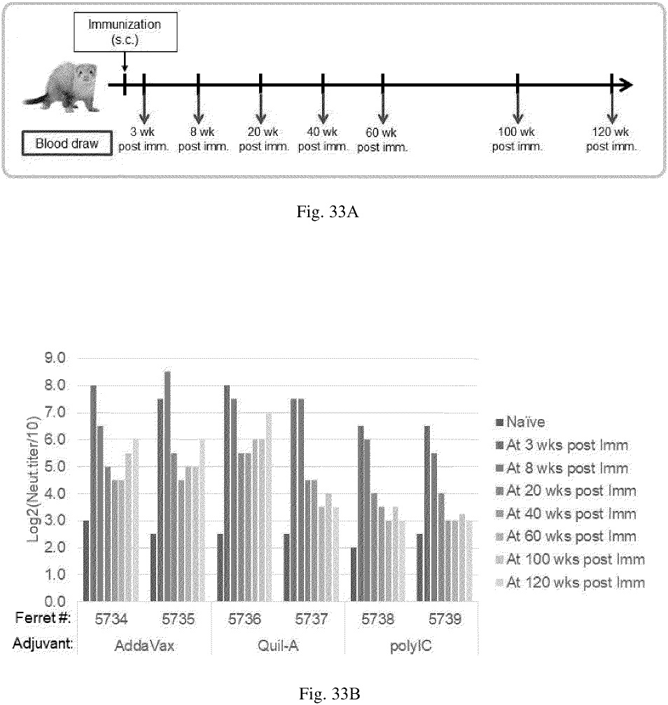

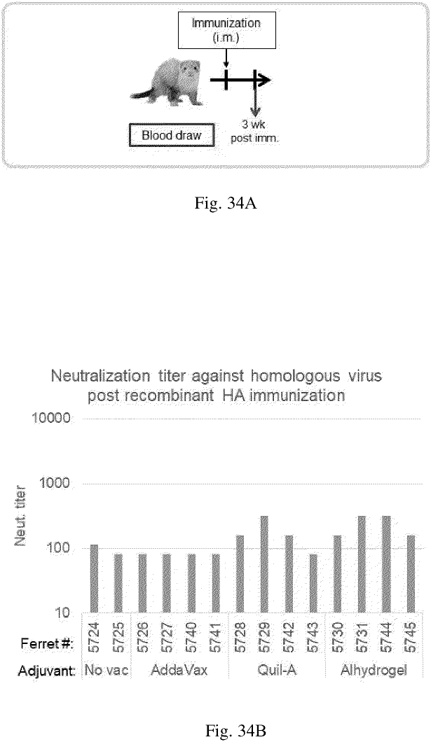

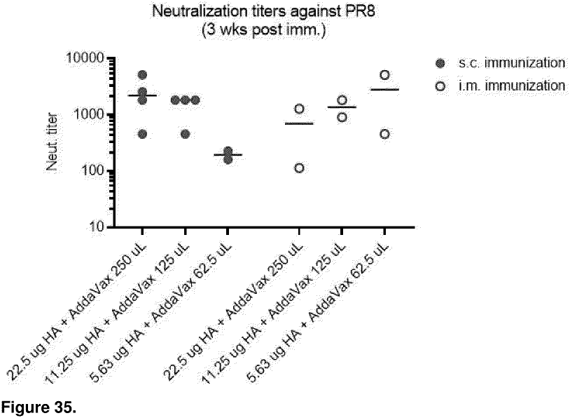

Streptavidin-coated VLPs were used to display biotinylated protein antigens such as the SARS-CoV-2 spike protein and DIII of the Zika virus envelope protein (Chiba et al., 2021; Castro et al., 2021). In this study those me VLPs are used to display the S2 subunit of the spike protein (FIG. 26b). The VLPs are based on the bacteriophage MS2 coat protein (Frietze et al., 2016); 90 MS2 coat protein homodimers self-assemble into an icosahedral structure (Valegard et al., 1994). BL21(DE3) Escherichia coli (E. Coli) was used to express a single chain dimer of the MS2 coat protein with an AviTag inserted in a surface loop that had been shown to tolerate peptide insertions (Table S1) (Peabody et al., 2008). The inserted AviTag allowed for site-specific biotinylation of each coat protein dimer. After expression, the VLPs were purified using Capto Core 700 resin and size exclusion chromatography (SEC). The VLPs were then biotinylated and subsequently separated from the biotinylation reagents using SEC. The biotinylated MS2 VLPs were added dropwise to a large excess of stirred streptavidin (SA), which had been expressed as inclusion bodies, refolded, and purified using iminobiotin affinity chromatography (Chiba et al., 2021; Castro et al., 2021). The resulting MS2-SA VLPs were separated from excess streptavidin using size exclusion chromatography. Consistent with prior characterization (Chiba et al., 2021), SDS-PAGE analysis of the MS2-SA VLPs indicated that there were approximately 70 streptavidin molecules bound to each MS2 biotin VLP (FIG. 32a). In addition, the MS2-SA VLPs were found to be pure and homogenous in size based on characterization by SEC (FIG. 26c), dynamic light scattering (DLS; FIG. 26d), negative-stain transmission electron microscopy (NS-TEM; FIG. 1e) and cryo-electron microscopy (cryo-EM; FIG. 26f).

Biotinylated S2 was next produced such that it could be displayed on the MS2-SA VLPs. Expi293F mammalian cells were used to express the HexaPro (Hseih et al., 2020) variant of the SARS-CoV-2 spike protein’s S2 subunit with an N-terminal signal peptide, a C-terminal trimerization domain to promote stability, a C-terminal AviTag for biotinylation, and a C-terminal his-tag for purification (Table S1). The expressed S2 was purified using immobilized metal affinity chromatography (IMAC) and was then biotinylated in vitro. The biotinylated S2 was separated from biotinylation reagents using size exclusion chromatography and could then be displayed on the MS2-SA VLPs.

To determine the appropriate ratio of S2 to add to MS2-SA VLPs, analytical SEC was used. Mixtures of the two proteins were made that contained a constant amount of S2 and varying amounts of MS2-SA VLPs. The ratio of the mixture with the least amount of MS2-SA VLPs that displayed no indication of excess S2 in an SEC chromatogram was determined to be the approximate stoichiometric ratio. Further analysis by SDS-PAGE indicated that this 18 stoichiometric ratio resulted in approximately S2 molecules conjugated to each MS2-SA VLP (FIG. 32b). The MS2-SA and biotinylated S2 were mixed in this ratio to create the VLP-S2 immunogen.

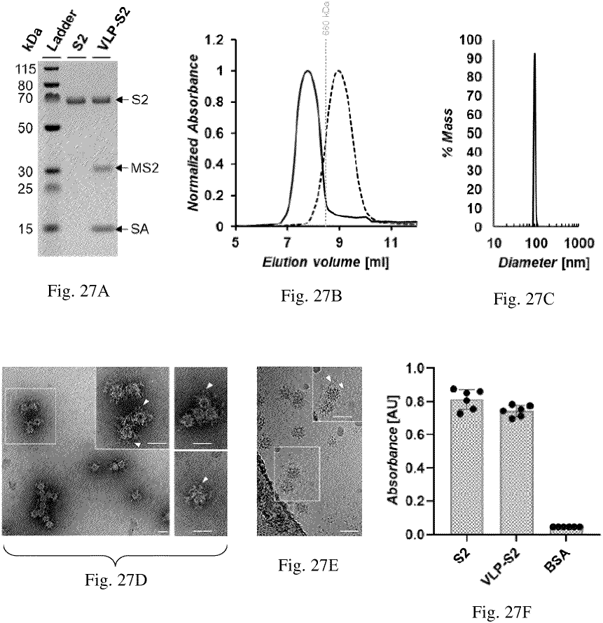

The VLP-S2 immunogen was characterized in vitro using several different bioanalytical techniques. First, the proteins that made up VLP-S2 were characterized by SDS polyacrylamide gel electrophoresis (SDS-PAGE), which indicated that the proteins were pure (FIG. 27a). In addition, comparison of the molecular weight ladder to the bands representing deglycosylated S2 (about 63 kDa), biotinylated MS2 (about 29 kDa), and monomeric streptavidin (about 15 kDa) demonstrated that these proteins aligned as expected with molecular weight standards. The VLP-S2 was also analyzed using analytical SEC, where chromatograms were generated for VLP-S2, S2 alone, and the molecular weight standard thyroglobulin (FIG. 27b). The resulting UV trace corresponding to the VLP-S2 contained a single peak that appeared before the peak for S2 alone. Therefore, the VLP-S2 was free of excess S2 and was generally uniform in size. To obtain a direct size measurement of the VLP-S2, we used Dynamic Light Scattering (DLS) (FIG. 27c), NS-TEM (FIG. 27d), and cryo-EM (FIG. 27e). The DLS measurements indicated that the VLP-S2 construct was approximately 90 nm in diameter. Characterization of the VLP-S2 by NS-TEM and cryo-EM confirmed the presence and coating of the S2 protein on the surface of the MS2-SA VLP. NS-TEM analysis suggested that VLP-S2 was about 65 nm in diameter on average (n=300). The larger size indicated by DLS may be a result of the fact that scattering intensity is proportional to the sixth power of the radius, giving rise to a disproportionately higher weighting of larger particles. We next used ELISA to probe the binding of the anti-S2 monoclonal antibody 0304-3H3 (Chi et al., 2020) to S2 and VLP-S2 (FIG. 27f). This antibody bound to both the S2 and VLP-S2, suggesting that S2 retained its antigenicity after conjugation to VLPs.

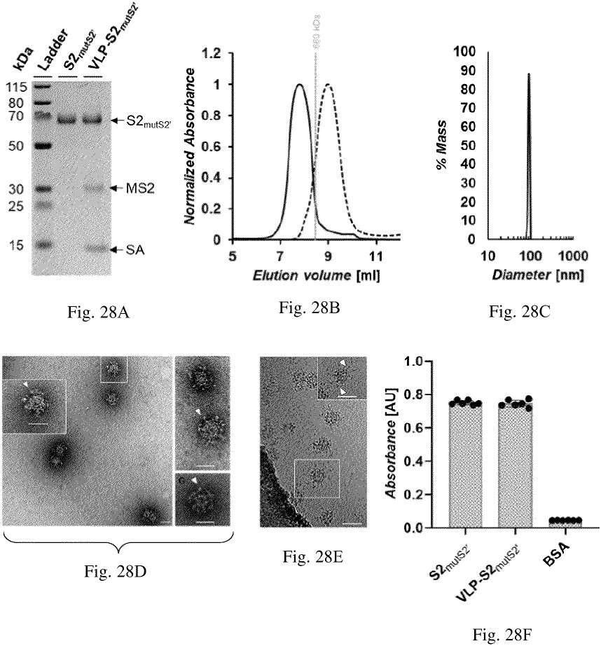

In addition to the VLP-S2, VLP-S2_{mutS2′} particles were generated. The VLP-S2_{mutS2′} displayed an S2 variant (S2_{mutS2′}) that contained S2′ cut site residues that had been mutated to glycine residues (Table 7). The purpose of this mutation was to prevent potential proteolytic cleavage of the S2 immunogen at the S2′ cut site. The VLP-S2_{mutS2′} was generated and characterized using the same procedures described above for the VLP-S2 (FIG. 28).

TABLE 7

Protein sequences

| | |
|---|---|
| MS2-AviTag | MASNFTQFVLVDNGGTGDVTVAPSNFANGVAEWISSNSRSQAYKVTCSVRQSSAQNRK YTIKVEVPKVATQTVGGVELPVAAWRSYLNMELTIPIFATNSDCELIVKAMQGLLKDG NPIPSAIAANSGIYASNFTQFVLVDNGGGLNDIFEAQKIEWHETGDVTVAPSNFANGV AEWISSNSRSQAYKVTCSVRQSSAQNRKYTIKVEVPKVATQTVGGVELPVAAWRSYLN MELTIPIFANTNSDCELIVKAMQGLLKDGNPIPSAIAANSGIY (SEQ ID NO: 14 ) |
| S2 | METDTLLLWVLLLWVPGSTGDSVASQSIIAYTMSLGAENSVAYSNNSIAIPTNFTISV TTEILPVSMTKTSVDCTMYTCGDSTECSNLLLQYGSFCTQLNRALTGIAVEQDKNTQE VFAQVKQIYKTPPIKDFGGFNFSQILJPDPSKPSKRSPIEDLLFNKVTLADAGFIKQYG DCLGDIAARDLICAQKFNGLTVLPPLLTDEMIAQYTSALLAGTITSGWTFGAGPALQI PFPMQMAYRFNGIGVTQNVLYENQKLIANQFNSAIGKIQDSLSSTPSALGKLQDVVNQ NAQALNTLVKQLSSNFGAISSVLNDILSRLDPPEAEVQIDPLITGPLQSTQTYVTQQL IRAAEIRASANLAATKMSECVLGQSKRVDFCGKGYHLMSFPQSAPHGVVFLHVTYVPA QEKNFTTAPAICHDGKAHFPREGVFVSNGTHWFVTQRNFYEPQIITTDNTFVSGNCDV VIGTVNNTVYDPLQPELDSFKEELDKYFKNHTSPDVDLGDISGINASVVNIQKEIDRL NEVAKNLNESLIDLQELGKYEQGSGYIPEAPRDGQAYVRKDGEWVLLSTFLGGLNDIF EAQKIEWHEHHHHHH (SEQ ID NO:15) |
| S2_{mutS2′} | METDTLLLWVLLLWVPGSTGDSVASQSIIAYTMSLGAENSVAYSNNSIAIPTNFTISV TTEILPVSMTKTSVDCTMYICGDSTECSNLLLQYGSFCTQLNRALTGIAVEQDKNTQE VFAQVKQIYKTPPIKDFGGFNFSQILPDPSKPSGGSPIEDLLFNKVTLADAGFIKQYG DCLGDIAARDLICAQKFNGLTVLPPLLTDEMIAQYTSALLAGTITSGWTFGAGPALQI PFPMQMAYRFNGIGVTQNVLYENQKLIANQFNSAIGKIQDSLSSTPSALGKLQDVVNQ NAQALNTLVKQLSSNFGAISSVLNDILSRLDPPEAEVQIDRLITGRLQSLQTYVTQQL IRAAEIRASANLAATKMSECVLGQSKRVDFCGKGYHLMSFPQSAPHGVVFLHVTYVPA QEKNFTTAPAICHDGKAHFPREGVFVSNGTHWFVTQRNFYEPQIITTDNTFVSGNCDV VIGIVNNTVYDPLQPELDSFKEELDKYFKNHTSPDVDLGDISGINASVVNIQKEIDRL NEVAKNLNESLIDLQELGKYEQGSGYIPEAPRDGQAYVRKDGEWVLLSTFLGGLNDIF EAQKIEWHEHHHHHH (SEQ ID NO:16) |

### Multivalent S2-based Vaccines Protect Against SARS-CoV-2

The protective efficacy of the VLP-S2 and VLP-S2_{mutS2′} against an early isolate of SARS-CoV-2, SARS-CoV-2/UT-NCGM02/Human/2020/Tokyo (NCGM02; spike protein with aspartate (D) at position 614 (Imai et al., 2020) (FIG. 29a), was assessed. Hamsters were immunized with either VLP-S2, VLP-S2_{mutS2′}, or MS2-SA VLP (VLP-control) alone, adjuvanted with Addavax, and were boosted 28 days later. Hamsters immunized with the VLP-S2 and VLP-S2_{mutS2′} generated high IgG antibody titers against the S ectodomain (FIG. 29b; Table 6). To gauge whether immunization with VLP-S2 and VLP-S2_{mutS2′} was protective, the vaccinated hamsters were intranasally inoculated with 10³ plaque-forming units (pfu) of NCGM02 (Imai et al., 2020) 51 days after the initial immunization. The hamsters were then sacrificed 3 days after infection and viral titers in their lungs and nasal turbinates were quantified by plaque assay. The mean viral titer in the lungs of hamsters immunized with VLP-S2 was nearly 100-fold lower than that of hamsters immunized with VLP-2 control (FIG. 29c). The mean viral titer in the lungs of hamsters immunized with VLP-S2_{mutS2′} was more than 7,000-fold lower than that of control immunized hamsters (FIG. 29c). These results 4 demonstrate that immunization with the S2-based immunogens VLP-S2 and VLP-S2_{mutS2′} provides protection against SARS-CoV-2. Characterization of viral titers in the nasal turbinates of the immunized hamsters also indicated that the multivalent S2 constructs provided protection against SARS-CoV-2 (FIG. 29d). The mean viral titers in the nasal turbinates of hamsters immunized with VLP-S2 and VLP-S2_{mutS2′} were respectively 3- and 36-fold lower than that of hamsters immunized with VLP-control.

### Multivalent S2-based Vaccines Elicit Cross-reactive Antibodies

Next, the breadth of the immune response generated by VLP-S2 and VLP-S2_{mutS2′} was evaluated using ELISA. Immunization with the multivalent S2 constructs elicited high antibody titers against the spike protein of not only the original Wuhan-Hu-1 SARS-CoV-2 (614D) (Wu et al., 2020), but also against the spike proteins of the SARS-CoV-2 variant B.1.351, SARS-CoV-1, and the four endemic human coronaviruses HKU-1, OC43, NL63, and 229E (FIG. 29e and Table 6). This 16 substantial cross-reactivity suggests that immunization with multivalent S2-based immunogens may be a promising strategy for eliciting a broadly protective response against coronaviruses.

### Characterization of the Protective Efficacy of VLP-S2_{mutS2′}

Given the better protective efficacy provided by VLP-S2_{mutS2′} than VLP-S2 (FIGS. 29c-d), it was decided to use the VLP-S2_{mutS2′} construct for further characterization against a previous variant of concern, B.1.617.2, delta variant (hCoV-19/USA/WI-UW-5250/2021) (Halfmann et al., 2022)44. The efficacy of VLP-S2_{mutS2′} against B.1.617.2 was assessed using a similar prime/boost immunization regimen with Addavax as the adjuvant as we first used against the early NCGM02 isolate (FIG. 29a). While there was a 35-fold and 2-fold decrease in the mean viral titers in nasal turbinate and lung tissues, respectively, for hamsters immunized with VLP-S2_{mutS2′} relative to controls, this difference was not statistically significant (FIG. 30a).

The effect of providing an extra vaccine dose, i.e., a third immunization with VLP-S2_{mutS2′}, while retaining Addavax as the adjuvant, was tested. Encouragingly, following a challenge with B.1.617.2, a statistically significant decrease was observed, e.g., about 90-fold decrease, in the mean lung titers of hamsters immunized with VLP-S2_{mutS2′} compared to those of control vaccinated hamsters and a about 11-fold decrease in the mean nasal turbinate titers relative to controls (FIG. 30b).

Having demonstrated the greater protection provided by an additional dose of the vaccine, the effect of different adjuvants, which can greatly influence the magnitude and quality of the immune response (Liang et al., 2020; Arunachalam et al., 2021; Ragupathi et al., 2011; Tewari et al., 2010), was tested. For these experiments, the efficacy of VLP-S2_{mutS2′} against B.1.617.2 was tested using a prime/boost immunization regimen (FIG. 29a), but using the adjuvants - QS-21, AddaS03 (a commercially available adjuvant system similar to GSK’s AS03) plus poly I:C (AS03 + pIC), and R848 (FIGS. 30c,d). A statistically significant decrease in lung titers was observed for hamsters immunized with VLP-S2_{mut2′} relative to controls, when using QS-21 or AS03 + pIC as adjuvants (33-fold and 127-fold, respectively; FIG. 30C). In contrast, no significant difference in lung titers was seen when using R848 as an adjuvant. A significant decrease in nasal turbinate titers for hamsters immunized with VLP-S2_{mutS2′} was observed relative to controls when using QS-21 or AS03 + pIC as adjuvants (about 18-fold and about 195-fold, respectively; FIG. 30d). Based on these results (FIGS. 5b-d), a three-dose immunization regimen, with a mixture of AS03 + pIC as adjuvants, was selected for further characterization of the breadth of protection. Immunization with VLP-S2_{mutS2′} Provides Broad Protection Against SARS-CoV-2 Variants of Concern and Pangolin Coronaviruses

The breadth of the antibody response elicited by the immunization regimen (3 doses; AS03 + pIC) was characterized by ELISA. Consistent with the high degree of conservation in the S2 domain, immunization with VLP-S2_{mutS2′} elicited high IgG antibody titers against early SARS-CoV-2 spike proteins (either with aspartate (D) or glycine (G) at position 614 [S-614D or S-614G, respectively), spike proteins of variants (B.1.617.2, B.1.351, BA.1 and BA.2), as well as against the spike proteins of other sarbecoviruses including a bat coronavirus (SARS-like coronavirus, RsSHC014), a pangolin coronavirus (Pg CoV) (BetaCoV/pangolin/Guandong/1/2019), and SARS-CoV-1 (FIG. 31a). Moreover, high IgG antibody titers were observed against the spike protein of the endemic human coronavirus NL63 (FIG. 27 31a).

Next, the ability of this immunization regimen to protect hamsters from a challenge with the SARS-CoV-2 variants, hCoV-19/USA/MD-HP01542/2021 (B.1.351, beta), hCoV-19/USA/WI-WSLH-221686/2021 (B.1.1.529 BA.1, Omicron), and B.1.617.2 (delta), was tested. For each variant challenge virus, a statistically significant decrease in lung titers (FIG. 31b) was observed for hamsters immunized with VLP-S2_{mutS2′} compared to control vaccinated hamsters. There was a greater than 6000-fold decrease in lung titers for hamsters immunized with VLP-S2_{mutS2′} relative to controls following a challenge with BA.1 (which shows extensive mutations in the S1 domain), highlighting the effectiveness of targeting the immune response towards the conserved S2 domain. A significant decreases in mean nasal turbinate titers (FIG. 31c) on day 3 was observed after infection with these variants for hamsters immunized with VLP-S2^{mutS2′} compared to control immunized hamsters.

The selected immunization regimen also provided excellent protection against a challenge with Pg CoV, BetaCoV/pangolin/Guandong/1/2019. Replicating Pg CoV was not detected in the lungs of vaccinated hamsters (limit of detection 1.3 log₁₀ pfu/g) while Pg CoV replicated to about 10⁵ to 10⁷ pfu/g in the lungs of control unvaccinated hamsters (FIG. 31d). In the nasal turbinates of hamsters, Pg CoV replicated better compared to the lungs, and vaccination with VLP-S2ₘᵤₜ reduced virus titers in the nasal turbinates by 100-fold (FIG. 31d).

Sera from hamsters immunized with VLP-S2_{mutS2′} using the selected immunization regimen showed weak neutralization activity in vitro against SARS-CoV-2/UT-HP095-1N/Human/2020/Tokyo, an early isolate with S-614D isolate in a focus reduction neutralization test (FRNT) assay with 50% reduction at a reciprocal serum dilution of about 35 (FIG. 31e). It was noted that other mechanisms, such as Fc effector functions may also contribute to the protection afforded to the hamsters through immunization with the multivalent S2 constructs. Fc effector functions have previously been identified as a mechanism by which S2-specific antibodies provide protection (Shiakolas et al., 2021). In addition, antibodies targeting the S2-analogous region of the influenza protein hemagglutinin (the stalk domain) are known to provide protection through Fc effector functions (DiLillo et al., 2014). The results demonstrate that the multivalent S2 constructs are capable of eliciting a broadly cross-reactive immune response that protects against multiple sarbecoviruses including an early isolate of SARS-CoV-2, SARS-CoV-2 variants (beta, delta, and omicron), and a pangolin coronavirus. Therefore, the S2 subunit should be considered in the development of next-generation coronavirus vaccines designed to protect against future SARS-CoV-2 variants and other zoonotic coronaviruses with pandemic potential.

In summary, a vaccine and data for an adjuvant and immunization regimen in Syrian hamsters and BALB/c mice are provided. The efficacy of the vaccine against SARS-CoV-2 variants and other coronaviruses was shown. In particular, immunization with S2-based constructs elicited a broadly cross-reactive IgG antibody response that recognized the spike proteins of not only SARS-CoV-2 variants, but also SARS-CoV-1, and the four endemic human coronaviruses. Importantly, immunization reduced virus titers in respiratory tissues in vaccinated animals challenged with SARS-CoV-2 variants B.1.351 (beta), B.1.617.2 (delta), and BA.1 (omicron) as well as a pangolin coronavirus. These results suggest that S2-based constructs can elicit a broadly cross-reactive antibody response resulting in limited virus replication thus providing a framework for designing vaccines that elicit broad protection against coronaviruses.

### Part B: Results

### Generation and in Vitro Characterization of S2 Nanoparticle-based Vaccines

Streptavidin-coated VLPs can be used to display biotinylated protein antigens such as the SARS-CoV-2 spike protein and DIII of the Zika virus envelope protein. Those VLPs were used to display the S2 subunit of the spike protein. The VLPs are based on the bacteriophage MS2 coat protein; 90 MS2 coat protein homodimers self-assemble into an icosahedral structure. BL21(DE3) Escherichia coli (E. Coli) were used to express a single chain dimer of the MS2 coat protein with an AviTag inserted in a surface loop that had been shown to tolerate peptide insertions (Table 7). The inserted AviTag allowed for site-specific biotinylation of each coat protein dimer. After expression, the VLPs were purified using Capto Core 700 resin and size exclusion chromatography (SEC). The VLPs were then biotinylated and subsequently separated from the biotinylation reagents using SEC. The biotinylated MS2 VLPs were added dropwise to a large excess of stirred streptavidin (SA), which had been expressed as inclusion bodies, refolded, and purified using iminobiotin affinity chromatography. The resulting MS2-SA VLPs were separated from excess streptavidin using size exclusion chromatography. Consistent with prior characterization, SDS-PAGE analysis of the MS2-SA VLPs indicated that there were approximately 70 streptavidin molecules bound to each MS2 biotin VLP (Fig. S1a). In addition, the MS2-SA VLPs were found to be pure and homogenous in size based on characterization by SEC, dynamic light scattering (DLS), negative-stain transmission electron microscopy (NS-TEM) and cryo-electron microscopy (cryo-EM).

Biotinylated S2 was next produced such that it could be displayed on the MS2-SA VLPs. We used Expi293F mammalian cells to express the HexaPro variant of the SARS-CoV-2 spike protein’s S2 subunit with an N-terminal signal peptide, a C-terminal trimerization domain to promote stability, a C-terminal AviTag for biotinylation, and a C-terminal his-tag for purification (Table 7). The HexaPro variant contains 6 stabilizing proline mutations, (F817P, A892P, A899P, A942P, K968P, and V969P), as reported by Hsieh et al. The expressed S2 was purified using immobilized metal affinity chromatography (IMAC) and was then biotinylated in vitro. The biotinylated S2 was separated from biotinylation reagents using size exclusion chromatography and could then be displayed on the MS2-SA VLPs.

To determine the appropriate ratio of S2 to add to MS2-SA VLPs, analytical SEC was used. Mixtures of the two proteins were made that contained a constant amount of S2 and varying amounts of MS2-SA VLPs. The ratio of the mixture with the least amount of MS2-SA VLPs that displayed no indication of excess S2 in an SEC chromatogram was determined to be the approximate stoichiometric ratio. Further analysis by SDS-PAGE indicated that this stoichiometric ratio resulted in approximately 30 S2 molecules conjugated to each MS2-SA VLP. The MS2-SA and biotinylated S2 were mixed in this ratio to create the VLP-S2 immunogen.

The VLP-S2 immunogen was characterized in vitro using several different bioanalytical techniques. First, the proteins that made up VLP-S2 were characterized by SDS polyacrylamide gel electrophoresis (SDS-PAGE), which indicated that the proteins were pure. In addition, comparison of the molecular weight ladder to the bands representing deglycosylated S2 (~63 kDa), biotinylated MS2 (~29 kDa), and monomeric streptavidin (~15 kDa) demonstrated that these proteins aligned as expected with molecular weight standards. The VLP-S2 was also analyzed using analytical SEC, where chromatograms were generated for VLP-S2, S2 alone, and the molecular weight standard thyroglobulin. The resulting UV trace corresponding to the VLP-S2 contained a single peak that appeared before the peak for S2 alone. Therefore, the VLP-S2 was free of excess S2 and was generally uniform in size. To obtain a direct size measurement of the VLP-S2, we used Dynamic Light Scattering (DLS), NS-TEM, and cryo-EM. The DLS measurements indicated that the VLP-S2 construct was approximately 90 nm in diameter. Characterization of the VLP-S2 by NS-TEM and cryo-EM confirmed the presence and coating of the S2 protein on the surface of the MS2-SA VLP. NS-TEM analysis suggested that VLP-S2 was ~65 nm in diameter on average (n=300). The larger size indicated by DLS may be a result of the fact that scattering intensity is proportional to the sixth power of the radius, giving rise to a disproportionately higher weighting of larger particles. ELISA was used to probe the binding of the anti-S2 monoclonal antibody 0304-3H3²¹ to S2 and VLP-S2. This antibody bound to both the S2 and VLP-S2, suggesting that S2 retained its antigenicity after conjugation to VLPs.

In addition to the VLP-S2, VLP-S2_{muts2′} particles were generated. The VLP-S2 displayed an S2 variant (S2_{mutS2′}) that contained S2′ cut site residues that had been mutated to glycine residues (Table S1). The purpose of this mutation was to prevent potential proteolytic cleavage of the S2 immunogen at the S2′ cut site. The VLP-S2 was generated and characterized using the same procedures described above for the VLP-S2.

### Multivalent S2-based Vaccines Reduce Coronavirus Titers in the Respiratory Tissues

The efficacy of the VLP-S2 and VLP-S2_{muts2′} against an early isolate of SARS-CoV-2, SARS-CoV-2/UT-NCGM02/Human/2020/Tokyo (NCGM02; spike protein with aspartate (D)at position 61445) was assessed. Hamsters were immunized with either VLP-S2, VLP-S2 VLP-S2, or MS2-SAVLP (VLP-control) alone, adjuvanted with AddaVax, and were boosted 28 days later. Hamsters immunized with the VLP-S2 and VLP-S2_{muts2′} generated high IgG antibody titers against the S ectodomain (Table 1). The vaccinated hamsters were intranasally inoculated with 103 plaque-forming units (pfu) of NCGM02⁴⁵ 51 days after the initial immunization. The hamsters were then sacrificed 3 days after infection and viral titers in their lungs and nasal turbinates were quantified by plaque assay. The mean viral titer in the lungs of hamsters immunized with VLP-S2 was nearly 100-fold lower than that of hamsters immunized with VLP-control. The mean viral titer in the lungs of hamsters immunized with VLP-S2 was more than 7,000-fold lower than that of control immunized hamsters. Characterization of viral titers in the nasal turbinates of the immunized hamsters also indicated that the multivalent S2 constructs provided partial protection against virus replication in the respiratory tissues. The mean viral titers in the nasal turbinates of hamsters immunized with VLP-S2 and VLP-S2 _{muts2′} were respectively 3- and 36-fold lower than that of hamsters immunized with VLP-control. It is possible that the increased efficacy seen with VLP-S2_{muts2′} is due to the proteolytic cleavage of the S2 construct at the S2′ site in vivo, after immunization.

### Multivalent S2-based Vaccines Elicit Cross-reactive Antibodies

Next, the breadth of the immune response generated by VLP-S2 and VLP-S2_{muts2′} was evaluated using ELISA. Immunization with the multivalent S2 constructs elicited high IgG antibody titers against the spike protein of not only the original Wuhan-Hu-1 SARS-CoV-2 (614D)52, but also against the spike proteins of the SARS-CoV-2 variant B.1.351, SARS-CoV-1, and the four endemic human coronaviruses HKU-1, OC43, NL63, and 229E. This substantial cross-reactivity suggests that immunization with multivalent S2-based immunogens may be a promising strategy for eliciting a broadly protective response against coronaviruses.

### Characterization of the Efficacy of VLP-S2_{muts2′}

Given the better efficacy provided by VLP-S2_{muts2′} than VLP-S2, the VLP-S2 _{muts2′} constructwas used for further characterization against a previous variant of concern, B.1.617.2, delta variant (hCoV-19NSA/WI-UW-5250/2021)⁵³. The efficacy of VLP-S2 _{muts2′} against B.1.617.2 was assessed using a similar prime/boost immunization regimen with AddaVax as the adjuvant as we first used against the early NCGM02 isolate. While there was a 35-fold and 2-fold decrease in the mean viral titers in nasal turbinate and lung tissues, respectively, for hamsters immunized with VLP-S2_{muts2′} relative to controls, this difference was not statistically significant.

The effect of providing an extra vaccine dose, i.e., a third immunization with VLP-S2 _{muts2′}, while retaining AddaVax as the adjuvant, was tested. Encouragingly, following a challenge with B.1.617.2, we now observed a statistically significant ~90-fold decrease in the mean lung viraltiters of hamsters immunized with VLP-S2_{muts2′} compared to those of control vaccinated hamsters and a ~ 11-fold decrease in the mean nasal turbinate viral titers relative to controls.

Having demonstrated the greater efficacy provided by an additional dose of the vaccine, the effect of different adjuvants, which can greatly influence the magnitude and quality of the immune response, was tested. For these experiments, the efficacy of VLP-S2_{muts2′} against B.1.617.2 was assessed using a prime/boost immunization regimen, but using the adjuvants - QS-21, AddaS03 (a commercially available adjuvant system similar to GSK’s AS03) plus poly I:C (AS03 + pIC), and R848. A statistically significant decrease in lung viral titers for hamsters immunized with VLP-S2 _{muts2′} was observed relative to controls, when using QS-21 or AS03 + pIC as adjuvants (33-fold and 127-fold, respectively). In contrast, no significant difference in lung viral titers was seen when using R848 as an adjuvant. We also observed a significant decrease in nasal turbinate viral titers for hamsters immunized with VLP-S2_{muts2′} relative to controls when using QS-21 or AS03 + pIC as adjuvants (~18-fold and ~195-fold, respectively). Based on these results, a three-dose immunization regimen, with a mixture of AS03 + pIC as adjuvants, was selected for further characterization of the breadth of protection.

### VLP-S2_{muts2′} Demonstrates Efficacy against Challenges with SARS-CoV-2 Variants of Concern and Pangolin Coronaviruses

The breadth of the antibody response elicited by the immunization regimen (3 doses; AS03 + pIC) was measured by ELISA. Consistent with the high degree of conservation in the S2 domain, immunization with VLP-S2_{muts2′} elicited high IgG antibody titers against early SARS-CoV-2 spike proteins (either with aspartate (D) or glycine (G) at position 614 [S-614D or S-614G, respectively), spike proteins of variants ( B.1.617.2, B.1.351, BA.1 and BA.2), as well as against the spike proteins of other sarbecoviruses including a bat coronavirus (SARS-like coronavirus, RsSHC014), a pangolin coronavirus (Pg CoV) (BetaCoV/pangolin/Guandong/1/2019), and SARS-CoV-1. Moreover, we also observed high IgG antibody titers against the spike protein of the endemic human coronavirus NL63.

Next, the efficacy of this immunization regimen against a challenge with the SARS-CoV-2 variants, hCoV-19/USA/MD-HP01542/2021 (B.1.351, beta), hCoV-19/USA/WI-WSLH-221686/2021 (B.1.1.529 BA.1, Omicron), and B.1.617.2 (delta), was tetsted. For each variant challenge virus, a statistically significant decrease in lung viral titers was observed for hamsters immunized with VLP-S2 compared to control vaccinated hamsters. There was a greater than 6000-fold decrease in lung viral titers for hamsters immunized with VLP-S2_{muts2′} relative to controls following a challenge with BA.1 (which shows extensive mutations in the S1 domain), highlighting the effectiveness of targeting the immune response towards the conserved S2 domain. A significant decrease in mean nasal turbinate viral titers on day 3 after infection with these variants was observed for hamsters immunized with VLP-S2_{muts2′} compared to control immunized hamsters.

The immunization regimen was also very effective at reducing viral titers in respiratory tissues following a challenge with Pg CoV, BetaCoV/pangolin/Guandong/1/2019. Replicating Pg CoV was not detected in the lungs of vaccinated hamsters (limit of detection 1.3 log10 pfu/g) while Pg CoV replicated to ~10⁵ to 10⁷ pfu/g in the lungs of control unvaccinated hamsters. In the nasal turbinates of hamsters, Pg CoV replicated better compared to the lungs, and vaccination with VLP-S2ₘᵤₜ reduced virus titers in the nasal turbinates by 100-fold.

Sera from hamsters immunized with VLP-S2_{muts2′} using the selected immunization regimen showed neutralization activity in vitro against SARS-CoV-2/UT-HP095-1N/Human/2020/Tokyo, an early S-614D isolate in a focus reduction neutralization test (FRNT) assay with 50% reduction at a reciprocal serum dilution of ~ 35.

### Immunization With VLP-S2_{muts2′} Demonstrates Efficacy in Mice Against a SARS-CoV-2 Challenge in Mice and Elicits a Broadly Neutralizing Antibody Response

Ng et al. reported a DNA-vaccine-based approach to elicit S2-targeted immunity in mice. Sera from S2-immunized mice demonstrated broad neutralizing activity in vitro. While this study did not demonstrate protection from a challenge with more recent variants of concern such as delta and BA.1, or with non-SARS-CoV-2 sarbecoviruses, SARS-CoV-2-S2-vaccinated K18-hACE2 mice challenged with SARS-CoV-2 Wuhan and Alpha isolates showed a 0.9 and 1.1 log reduction in SARS-CoV-2 E copies in the lungs on day 4, respectively, compared with unvaccinated controls. In the mouse study, while a mouse-adapted SARS-CoV-2 (an early Wuhan-like isolate) replicated to > 10⁷ pfu/g in the lungs of control unvaccinated BALB/c mice (FIG. 36a), we were unable to detect replication of the mouse-adapted virus in the lungs of vaccinated mice (limit of detection 1.3 log₁₀ pfu/g). Sera from mice obtained after a single immunization with VLP-S2_{muts2′} also showed neutralization activity in vitro against three SARS-CoV-2 variants and Pg-CoV in an FRNT assay (FIG. 36b).

### Discussion

Despite the high efficacy of licensed vaccines against the original SARS-CoV-2 strain, studies have shown reduced protection against recent variants, mostly due to the high number of mutations found in areas of the S protein targeted by these vaccines. As a result, there has been a growing interest in improving the breadth of protection provided by vaccines. While near-term interest is focused on vaccines that provide protection against SARS-CoV-2 variants (pan-SARS-CoV-2), there is also an interest in developing vaccines that protect against all sarbecoviruses (pan-sarbecovirus) and ultimately vaccines that provide protection against all betacoronaviruses (pan-betacoronavirus). There are two main strategies for designing vaccines that elicit a broadly protective antibody response. One strategy, which has been used successfully with seasonal influenza vaccines, is based on using a mixture of different antigens. For instance, groups are pursuing the design of nanoparticle-based coronavirus vaccines that use a mixture of different receptor-binding domain (RBD) antigens.Cohen et al. have reported the design of mosaic nanoparticle vaccines - vaccines displaying multiple RBDs on the same nanoparticle.59,60 Mosaic nanoparticles based on a mixture of 8 different clade 1 and clade 2 sarbecovirus RBD antigens protected against challenges with both SARS-CoV-1 and SARS-CoV-2 and also showed broad neutralization activity in vitro. Similarly, Walls et al. showed that mosaic nanoparticles based on a mixture of 4 different sarbecovirus RBDs could protect against a challenge with SARS-CoV-1, although the SARS-CoV RBD was not a component of the vaccine. While these results are promising and consistent with an approach that has worked with seasonal influenza vaccines, concerns include the large number of antigens required to provide broad protection as well as the plasticity of RBD domain and the possible emergence of new vaccine-evading RBD variants.

A second approach is to focus on parts of the S protein that are more conserved, which might enable the design of broadly protective vaccines without using mixtures of 4 or 8 antigens. To that end, immunogens based on the conserved S2 domain of SARS-CoV-2 were developed. The immunization regimen - three doses of the VLP-S2 _{muts2′} construct with AS03 + pIC as an adjuvant demonstrated efficacy in hamsters against challenges with SARS-CoV-2 variants as well as a pangolin coronavirus.

While sera from immunized animals in the study neutralized the viruses in vitro, other mechanisms, such as Fc effector functions, may also contribute to the efficacy provided by immunization with the multivalent S2 constructs. Fc effector functions have previously been identified as a mechanism by which S2-specific antibodies provide protection²⁹. In addition, antibodies targeting the S2-analogous region of the influenza protein hemagglutinin (the stalk domain) are known to provide protection through Fc effector functions.

The study primarily used hamsters to evaluate the immunogenicity and efficacy of our S2-based vaccine construct, because hamsters are naturally susceptible to infection by SARS-CoV-2 without the requirement of virus adaptation or the need of human ACE2 expressing transgenic hamsters. We show similar results for the S2-based vaccine construct in the mouse model using a mouse-adapted SARS-CoV-2 isolate. Vaccines based on nanoscaffolds are in clinical trials.

In summation, a vaccine based on the conserved S2 domain of SARS-CoV-2 (Table 8) was prepared and characterized and the immunogenicity and efficacy of this vaccine enhanced to significantly reduce virus replication in the respiratory tissues of vaccinated rodents. M multivalent S2 constructs are capable of eliciting a broadly cross-reactive immune response that protects against multiple sarbecoviruses including an early isolate of SARS-CoV-2, SARS-CoV-2 variants (beta, delta, and omicron), and a pangolin coronavirus.Thus, the S2 subunit may be employed in next-generation coronavirus vaccines designed to protect against future SARS-CoV-2 variants and other zoonotic coronaviruses with pandemic potential.

TABLE 8

S2 Sequence Homology.

| | SARS-CoV-2 S2_{mutS2′} | B.1.351 | B.1.617.2 | BA.1 | Pg-CoV |
|---|---|---|---|---|---|
| SARS-CoV-2 S2_{mutS2′} | 100.0% | 98.1% | 98.3% | 97.3% | 96.9% |
| B.1.351 | 98.1% | 100.0% | 99.4% | 98.5% | 98.1% |
| B.1.617.2 | 98.3% | 99.4% | 100.0% | 98.7% | 98.3% |
| BA.1 | 97.3% | 98.5% | 98.7% | 100.0% | 97.3% |
| Pg-CoV | 96.9% | 98.1% | 98.3% | 97.3% | 100.0% |

### REFERENCES

Addetia et al., J. Clin. Microbiol., ______:______ (2020).

Amanat et al., Nature Medicine, 26:1033 (2020).

Arsiwala et al., Nanoscale. 11:15307 (2019).

Arunachalam et al., Nature, 594:253 (2021).

Bachmann & Jennings, G. Nat. Rev. Immunol., 10:787 (2010)

Bachmann & Zinkemagel, Annu. Rev. Immunol., 15:235 (1997).

Baden et al., N. Engl. J. Med., 384:403 (2021).

Booth et al., J. Vis. Exp., ______:______ (2011).

Bruun et al., ACS Nano, 12:8855 (2018).

Castro et al., Adv. Healthc. Mater., e2002140, (2021).

Chi et al., Science, 369:650 (2020).

Chiba et al.,______, ______:______ (2020).

Chiba et al., Commun. Biol., 4:______ (2021).

Chiba et al., Immunization, (2020).

Cohen et al., Science, 371:735 (2021).

Corbett et al., Nature, 586:567 (2020).

DiLillo et al., Nat. Med., 20:143 (2014).

Dunkle et al., N. Engl. J. Med., 386:531 (2021).

Fairhead et al., J. Mol. Biol., 426:199 (2014).

Frietze et al., Curr. Opin., Virol., 18:44 (2016).

Galaz-Montoya et al., J. Struct. Biol., 190:279 (2015).

Garcia-Beltran et al., Cell, 184:2372 (2021).

Halfmann et al., Cell Reports, 38:_ (2022).

He et al., Nat. Immun., 23:960 (2022).

Hoffmann et al., Cell, 185:447 (2022).

Howarth & Ting, Nat. Protoc., 3:534 (2008).

Hsieh et al., Science, 369:1501 (2020).

Hu et al., Cell. & 18 Mol. Immunol., 19:644 (2022).

Imai et al., Proc. Natl. Acad. Sci. U.S.A., 117:16587 (2020).

Jennewein et al., bioRxiv, ______:______ gal(2021).

Jennewein et al., Cell Rep., 36:_ (2021).

Jia et al.. NPJ Vaccines, 6:47, (2021).

Ju et al., Nature, 584:115 (2020).

Jung & Mun, J. Vis. Exp., ______:______ (2018).

Kang et al., ACS Nano., 15:2738 (2021).

Khan et al., medRxiv, ______:______ (2022).

Krammer, Nature, 586:516 (2020).

Ladner et al., Cell. Rep. Med., 2:100189 (2021).

Lan et al., Nature. 581:215 (2020).

Lederer et al., Immunity, 53:1281 (2020).

Li et al., Cell Rep., 38:110210 (2022).

Liang et al., Front. Immunol., 11:______ (2020).

Lv et al., Science, 369:1505 (2020).

Madhi et al., N. Engl. J. Med., 15:384 (2021).

Madhi et al., N. Engl. J. Med., 384:1885 (2021).

Mastronarde, J. Struct. Biol. 152:36 (2005).

Matsuyama et al., Proc. Natl. Acad. Sci., 117:7001 (2020).

Menachery et al., Nat. Med., 21:1508 (2015).

Menachery et al., Proc. Natl. Acad. Sci. U.S.A., 113:3048 (2016).

Nguyen-Contant et al., mBio, 11:______ (2020).

Peabody et al., J. Mol. Biol., 380:252 (2008).

Pinto et al., bioRxiv, (2021).

Pinto et al., Nature, 583:290 (2020).

Pinto et al., Science, 373:1109 (2021).

Plummer & Manchester. Wiley Interdiscip. Rev. Nanomed. Nanobiotechnol., 3;174 (2011).

Polack et al., N. Engl. J. Med., 383:2603 (2020).

Ragupathi et al., Exp. Rev. Vaccines, 10:463 (2011).

Ravichandran et al., Sci. Transl. Med., 12:______ (2020).

Sadoff et al., N. Engl. J. Med., ______:______ (2021).

Sadoff et al., N. Engl. J. Med., 8, (2021).

Sanchez-Felipe et al., Nature, 590:320 (2021).

Sauer et al., Nat. Struct. Mol. Biol., 32:_ (2021).

Sauer et al., Nat. Struct. Mol. Biol., 48, (2021).

Saunders et al., Nature (2021).

Sheridan, Nature Biotechnology, 39:393 (2021).

Shi et al., Nature, 584:120 (2020).

Shiakolas et al., Cell. Rep. Med., (2021).

Shiakolas et al., Cell. Rep. Med., ______:100313 (2021).

Shin et al., Nat. Nanotechnol., 15:646 (2020).

Sia et al., Nature, 583:834 (2020).

Song et al., bioRxiv, (2020).

Song et al., Nat. Commun., 12:2938 (2021).

Tada et al., bioRxiv, (2021).

Tan et al., Nature Communications, 12:542 (2021).

ter Meulen et al.. PLoS Med., 3:e237 (2006).

Tewari et al., Vaccine, 28:7256 (2010).

Thuluva et al., medRxiv, ______:______ (2022).

Tostanoski et al, Nat. Med., 26:1694 2020).

Tuekprakhon et al., Cell ______:______ (2022).

Valegard et al., Nature, 371:623 (1994).

Voysey et al., The Lancet, 397:99 (2021).

Walls et al., Cell, 183:1735 (2020).

Walls et al., Cell., ______:______ (2020).

Walls et al., Nature, 531:114 (2016).

Walls et al., Nature, 531:28 (2016).

Wang et al., Nat. Commun., 12:1715 (2021).

Wang et al., Nat. Commun., 12:1715 (2021).

Wang et al., Nature, 593:130 (2021).

WHO Coronavirus (COVID-19) Dashboard, https://covid19.who.int/ (2021).

WHO Coronavirus (COVID-19) Dashboard. World Health Organization. https://covid19.who.int/. Published 14 2021. Updated Jun. 24, 2021. Accessed Jun. 24, 2021.

WHO COVID-19 Dashoard (2020).

Wrapp et al., Science, 367:1260 (2020).

Wu et al., Nature, 579:265 (2020).

Xu et al., Res., ______:______ (2022).

Yang et al., J. Struct. Biol., ______:______ (2021).

Yang et al., Nat. Commun., 11:4541 (2020).

Yuan et al., Science, 368:630 (2020).

Zhang et al., Sci. Rep., 10:18149, (2020).

Zheng et al., Nat. Methods, 14:331 (2017).

Zhou et al., bioRxiv, (2021).

Zhou et al., bioRxiv, ______:______ (2022B).

Zhou et al., Sci. Transl. Med., 14:16 (2022A).

All publications, patents and patent applications are incorporated herein by reference. While in the foregoing specification, this invention has been described in relation to certain preferred embodiments thereof, and many details have been set forth for purposes of illustration, it will be apparent to those skilled in the art that the invention is susceptible to additional embodiments and that certain of the details herein may be varied considerably without departing from the basic principles of the invention.

What is claimed is:

1. A nanoparticle that a) displays a coronavirus spike protein or a portion thereof on its surface, wherein the nanoparticle comprises a first fusion polypeptide comprising a first coat protein of a Fiersviridae and a second coat protein of a Fiersviridae comprising a biotinylated first peptide, wherein the first fusion polypeptide binds to a second fusion polypeptide via a tetravalent streptavidin molecule, wherein the second fusion polypeptide comprises the coronavirus spike protein or portion thereof, a second biotinylated peptide, and optionally a trimerization domain;
b) that displays a coronavirus spike protein or a portion thereof on its surface, wherein the nanoparticle comprises a first fusion polypeptide comprising a self-assembling protein and a first portion of a first protein that binds a second portion of the first protein, wherein the first fusion polypeptide binds to a second fusion polypeptide, wherein the second fusion polypeptide comprises the coronavirus spike protein or portion thereof and the second portion of the protein; or
c) a that displays a HA protein or a portion thereof on its surface in its native conformation, wherein the nanoparticle comprises a first fusion polypeptide comprising a self-assembling protein and a first portion of a first protein that binds a second portion of the first protein, wherein the first fusion polypeptide binds to a second fusion polypeptide, wherein the second fusion polypeptide comprises the HA protein or portion thereof and the second portion of the protein.

2. The nanoparticle of claim 1 which comprises an Emesvirus coat protein.

3. (canceled)

4. The nanoparticle of claim 1 wherein the spike protein comprises the ectodomain, the S2 domain or the receptor binding domain.

5-6. (canceled)

7. The nanoparticle of claim 1 wherein the particle has a diameter of about 50 to 100 nm.

8. The nanoparticle of claim 1 wherein the first peptide is an AviTag, a portion of avidin or streptavidin, a portion of neutravidin or a biotin carboxy carrier protein (BCCP, a biotin acceptor protein) or a portion thereof.

9. The nanoparticle of claim 1 wherein the second peptide is an AviTag.

10. The nanoparticle of claim 1 wherein the streptavidin comprises a tetravalent streptavidin or SAe4, SA4, Tre4, DTAg4, D4 or SAe1D3.

11. The nanoparticle of claim 1 wherein the first biotinylated peptide is C-terminal to the coat protein.

12. The nanoparticle of claim 1 wherein the first biotinylated peptide is N-terminal to the coat protein.

13. The nanoparticle of claim 1 wherein the trimerization domain is C-terminal to the coat protein.

14. The nanoparticle of claim 1 wherein the trimerization domain is N-terminal to the coat protein.

15. The nanoparticle of claim 1 wherein the second biotinylated peptide is C-terminal to the spike protein or portion thereof.

16. The nanoparticle of claim 1 wherein the second biotinylated peptide is N-terminal to the spike protein or portion thereof.

17. A method of making a nanoparticle that displays a coronavirus spike protein or a portion thereof or a HA protein or a portion thereof on its surface, comprising:
a) providing a first composition comprising isolated coat protein of Fiersviridae comprising a first biotinylated peptide which is bound to a tetravalent streptavidin;
b) providing a second composition comprising isolated protein comprising coronavirus spike protein or a portion thereof comprising a second biotinylated peptide and optionally a trimerization domain; and
c) combining an amount of the first composition and an amount of the second composition thereby yielding a nanoparticle comprising a diameter of about 50 to about 100 nm that displays a coronavirus spike protein or a portion thereof; or
d) providing a first composition comprising isolated coat protein of Fiersviridae comprising a first biotinylated peptide;
e) providing a second composition comprising isolated protein comprising spike protein or a portion thereof comprising a second biotinylated which is bound to a tetravalent streptavidin and optionally a trimerization domain; and
f) combining an amount of the first composition and an amount of the second composition thereby yielding a nanoparticle comprising a diameter of about 50 to about 100 nm that displays a coronavirus spike protein or a portion thereof; or
g) providing a first composition comprising isolated coat protein of Fiersviridae comprising a first biotinylated peptide;
h) providing a second composition comprising a tetravalent streptavidin;
i) providing a third composition comprising isolated protein comprising a coronavirus spike protein or a portion thereof comprising a second biotinylated peptide and optionally a trimerization domain; and
j) combining an amount of the first composition, an amount of the second composition and an amount of the third composition, thereby yielding a nanoparticle of about 50 to about 100 nm that displays a coronavirus spike protein or a portion thereof; or
k) providing a first composition comprising isolated first fusion polypeptide comprising a self-assembling protein and a first portion of a first protein that binds a second portion of the protein;
l) providing a second composition comprising isolated second fusion polypeptide comprises the coronavirus spike protein or portion thereof, and the second portion of the protein; and
m) combining an amount of the first composition and an amount of the second composition thereby yielding a nanoparticle comprising a diameter of about 50 to about 100 nm that displays a coronavirus spike protein or a portion thereof; or
n) providing a first composition comprising isolated coat protein of Fiersviridae comprising a first biotinylated peptide which is bound to a tetravalent streptavidin;
o) providing a second composition comprising isolated protein comprising HA protein or a portion thereof and a second biotinylated peptide and optionally comprising a heterologous trimerization domain; and
p) combining an amount of the first composition and an amount of the second composition thereby yielding a nanoparticle comprising a diameter of about 50 to about 100 nm that displays a HA protein or a portion thereof in a native orientation; or
q) providing a first composition comprising isolated coat protein of Fiersviridae comprising a first biotinylated peptide:
r) providing a second composition comprising isolated protein comprising HA protein or a portion thereof and a second biotinylated which is bound to a tetravalent streptavidin and optionally comprising a trimerization domain; and
s) combining an amount of the first composition and an amount of the second composition thereby yielding a nanoparticle comprising a diameter of about 50 to about 100 nm that displays a HA protein or a portion thereof in a native orientation; or
t) providing a first composition comprising isolated coat protein of Fiersviridae comprising a first biotinylated peptide;
u) providing a second composition comprising a tetravalent streptavidin;
v) providing a third composition comprising isolated protein comprising a HA protein or a portion thereof comprising a second biotinylated peptide and optionally comprising a trimerization domain; and
w) combining an amount of the first composition, an amount of the second composition and an amount of the third composition, thereby yielding a nanoparticle of about 50 to about 100 nm that displays a HA protein or a portion thereof in a native orientation.

18-26. (canceled)

27. A vaccine comprising the nanoparticle of claim 1.

28. A method to immunize a mammal, comprising administering to the mammal an effective amount of a composition having a plurality of the nanoparticles of claim 1.

29. The method of claim 28 wherein the mammal is a human.

30. The method of claim 28 wherein the composition further comprises an adjuvant.

31. (canceled)

32. The nanoparticle of claim 1 wherein the first protein is a fibronectin binding protein and the first protein is PilinC, RrgA, or Cpe0147.

33-58. (canceled)

59. A pharmaceutical composition comprising a single dose of immunogen comprising nanoparticles that display an influenza hemagglutinin (HA) protein or a portion thereof on its surface in a native orientation, wherein the nanoparticles comprise a first fusion polypeptide comprising a first coat protein of a Fiersviridae and a second coat protein of a Fiersviridae comprising a biotinylated first peptide, wherein the first fusion polypeptide binds to a second fusion polypeptide via a tetravalent streptavidin molecule, wherein the second fusion polypeptide comprises the HA protein or portion thereof, a second biotinylated peptide, and optionally a trimerization domain.

60-107. (canceled)
